(19) United States
(12) Patent Application Publication
(10) Pub. No.: US 2024/0150294 A1
(43) Pub. Date: May 9, 2024

(54) ESTROGEN RECEPTOR ALPHA ANTAGONISTS AND USES THEREOF

(71) Applicants: The University of Chicago, Chicago, IL (US); Loyola University of Chicago, Maywood, IL (US)

(72) Inventors: Sean W. Fanning, Riverside, IL (US); Geoffrey L. Greene, Chicago, IL (US); David J. Hosfield, Forest Park, IL (US)

(73) Assignees: The University of Chicago, Chicago, IL (US); Loyola University of Chicago, Maywood, IL (US)

(21) Appl. No.: 18/546,525

(22) PCT Filed: Feb. 17, 2022
(86) PCT No.: PCT/US2022/016813

§ 371 Date: Aug. 15, 2023

Related U.S. Application Data
(60) Provisional application No. 63/151,479, filed on Feb. 19, 2021.

(51) Int. Cl. C07D 221/20; A61K 45/06; A61P 35/00; C07D 401/12

(52) U.S. Cl. CPC C07D 221/20; A61K 45/06; A61P 35/00; C07D 401/12

(57) ABSTRACT

Provided is a compound of formula (I) or a pharmaceutically acceptable salt thereof, in which R¹, R², R³, R⁴, R⁵, X¹, X², and n are described herein. Also provided is a method of treating an estrogen-mediated disease requiring inhibition of estrogen receptor alpha (ER-alpha), such as cancer, in a subject or inhibiting ER-alpha in a cell with the compound of formula (I) or a pharmaceutically acceptable salt thereof.

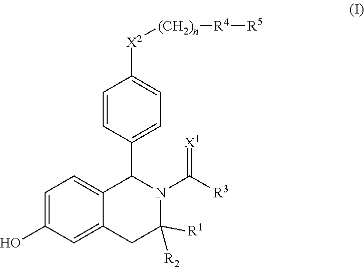

### CROSS-REFERENCE TO A RELATED APPLICATION

This patent application claims the benefit of U.S. Provisional Patent Application No. 63/151,479, filed Feb. 19, 2021, which is incorporated by reference herein in its entirety.

### BACKGROUND OF THE INVENTION

One in eight women will be diagnosed with breast cancer in her lifetime, around 240,000 women per year in the United States. Around 70% of primary breast cancers are histologically classified by their overexpression of estrogen receptor alpha (ERα). Typically, such patients will be prescribed antiestrogens to prevent primary disease metastasis following primary interventions. These antiestrogens are given as enduring therapies for 5-15 years. Competitive antiestrogens, such as tamoxifen and fulvestrant, are effective antiestrogens but suffer from pharmacologic issues that can limit their efficacies. Moreover, acquired somatic mutations to the ESR1 (ERα) gene can limit tamoxifen's efficacy.

Thus, there remains a need to provide additional antiestrogen agents to treat breast cancer and other estrogen-mediated indications including osteoporosis, vulvovaginal atrophy, hormone-replacement therapy, endometriosis, and osteo-arthritis.

### BRIEF SUMMARY OF THE INVENTION

The invention provides a compound of formula (I) or a pharmaceutically acceptable salt thereof:

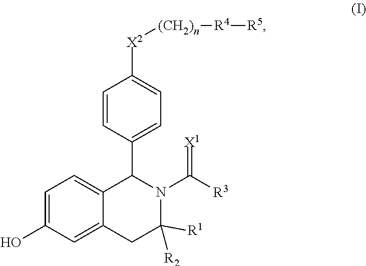

wherein R¹, R², R³, R⁴, R⁵, X¹, X², and n are described herein.

Also provided is a method of treating an estrogen-mediated disease (e.g., breast cancer) comprising administering to a subject in need thereof the compound of formula (I) or a pharmaceutically acceptable salt thereof.

Further provided is a method of inhibiting estrogen receptor alpha (ERa) in a cell comprising contacting the cell with the compound of formula (I) or a pharmaceutically acceptable salt thereof.

Additional embodiments are as described herein.

BRIEF DESCRIPTION OF THE SEVERAL VIEWS OF THE DRAWINGS

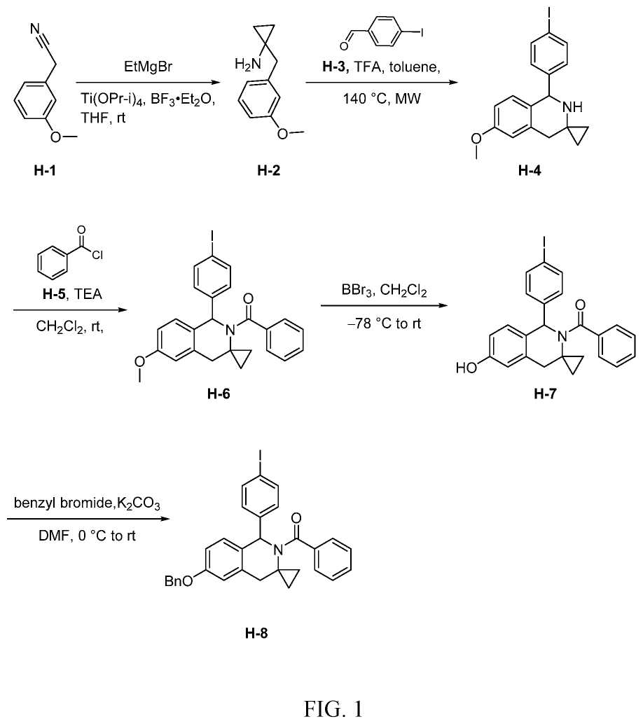

FIG. 1 is a chemical scheme of preparing compound H-8 starting from 2-(3-methoxyphenyl)acetonitrile (compound H-1).

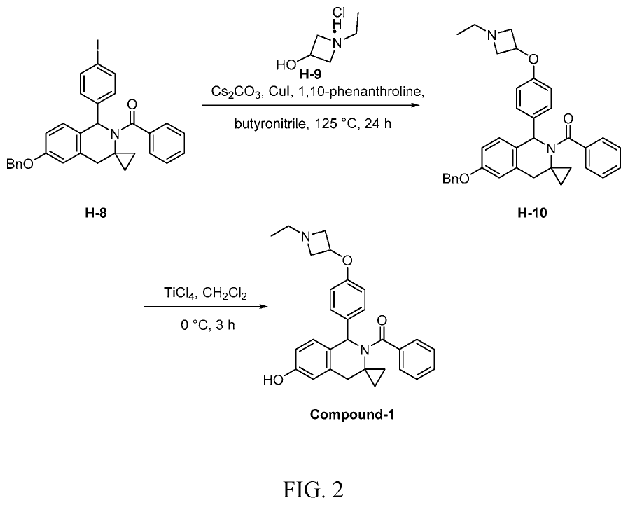

FIG. 2 is a chemical scheme of preparing compound 1 from H-8.

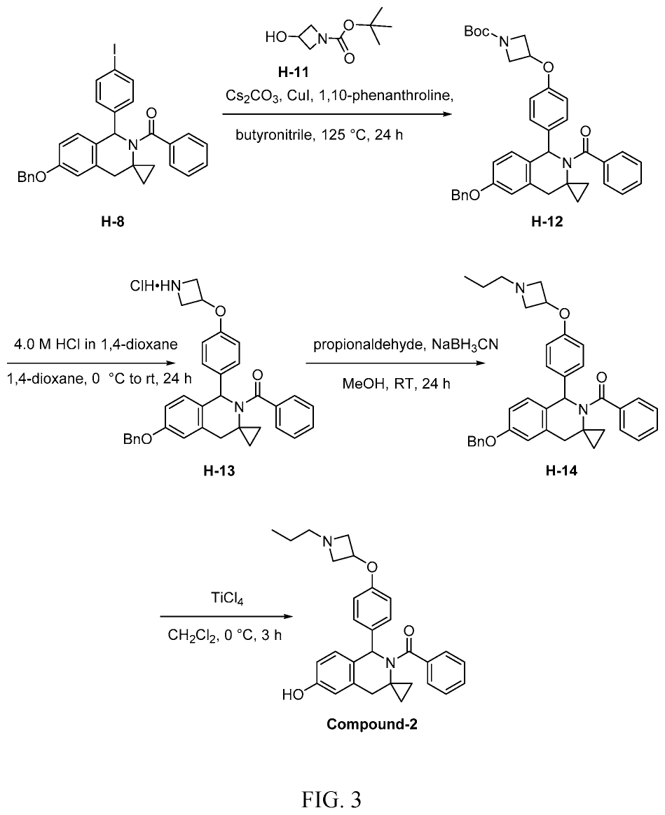

FIG. 3 is a chemical scheme of preparing compound 2 from H-8.

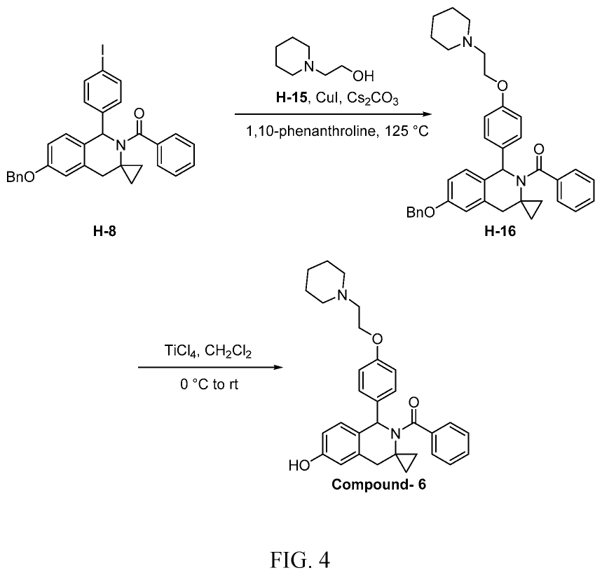

FIG. 4 is a chemical scheme of preparing compound 6 from H-8.

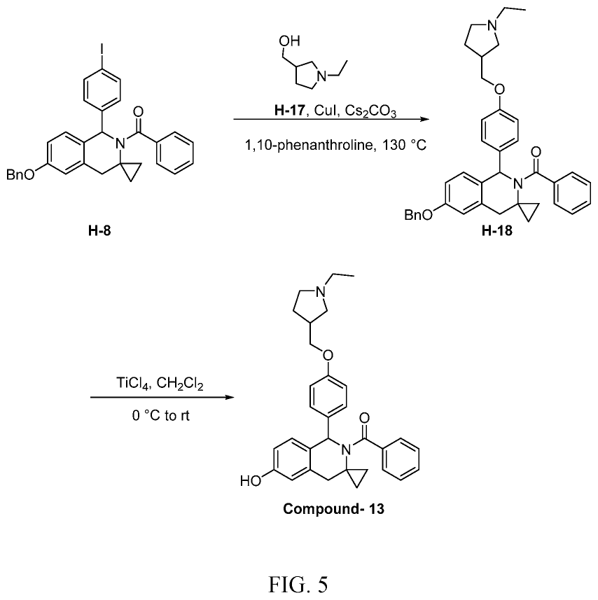

FIG. 5 is a chemical scheme of preparing compound 13 from H-8.

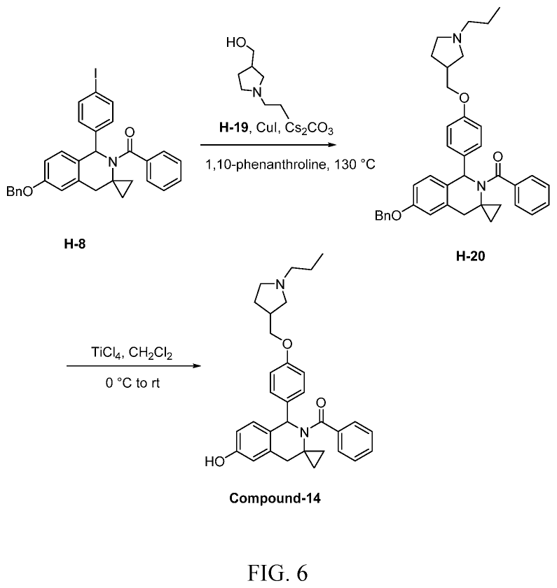

FIG. 6 is a chemical scheme of preparing compound 14 from H-8.

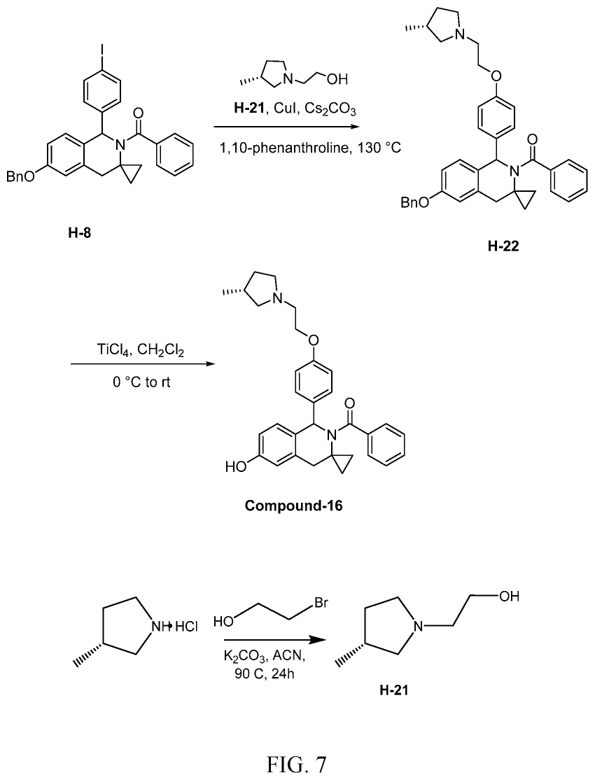

FIG. 7 is a chemical scheme of preparing compound 16 from H-8.

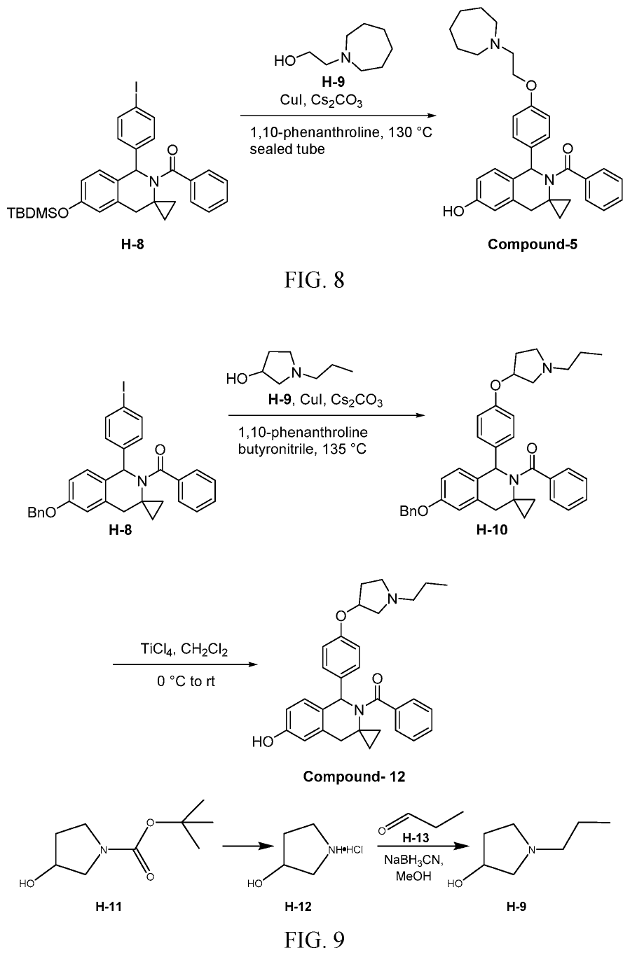

FIG. 8 is a chemical scheme of preparing compound 5 from H-8.

FIG. 9 is a chemical scheme of preparing compound 12 from H-8.

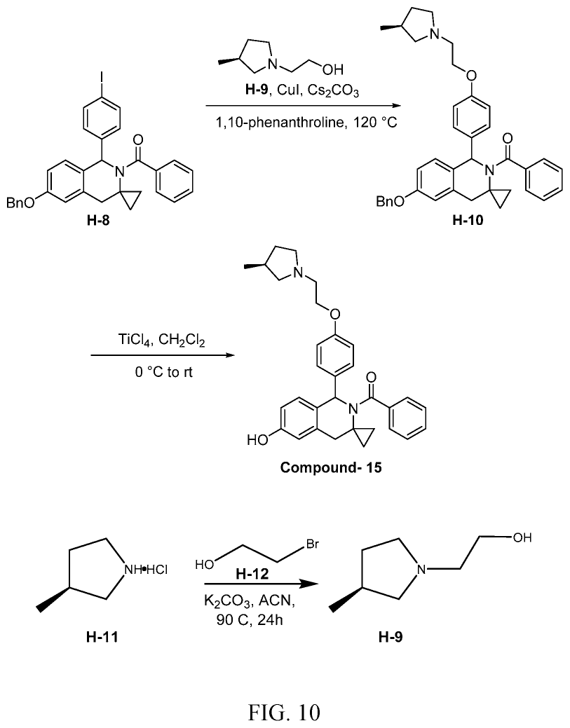

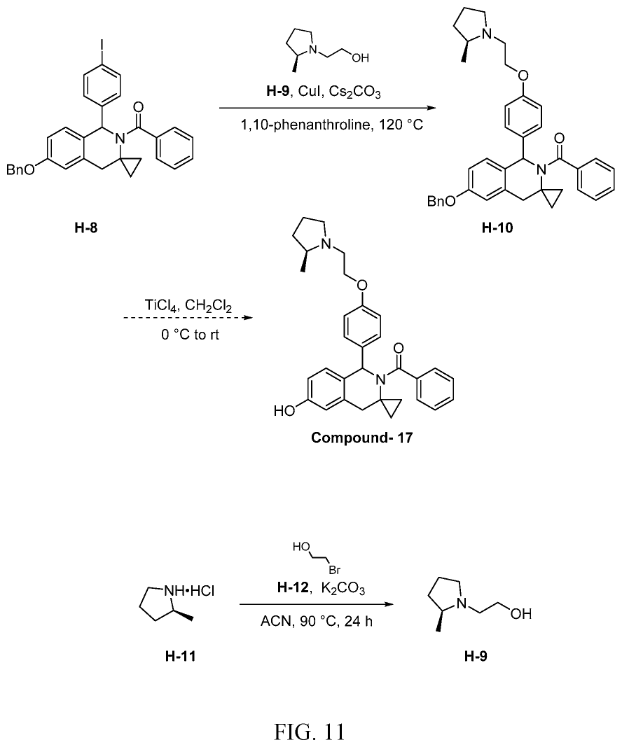

FIG. 10 is a chemical scheme of preparing compound 15 from H-8.

FIG. 11 is a chemical scheme of preparing compound 17 from H-8.

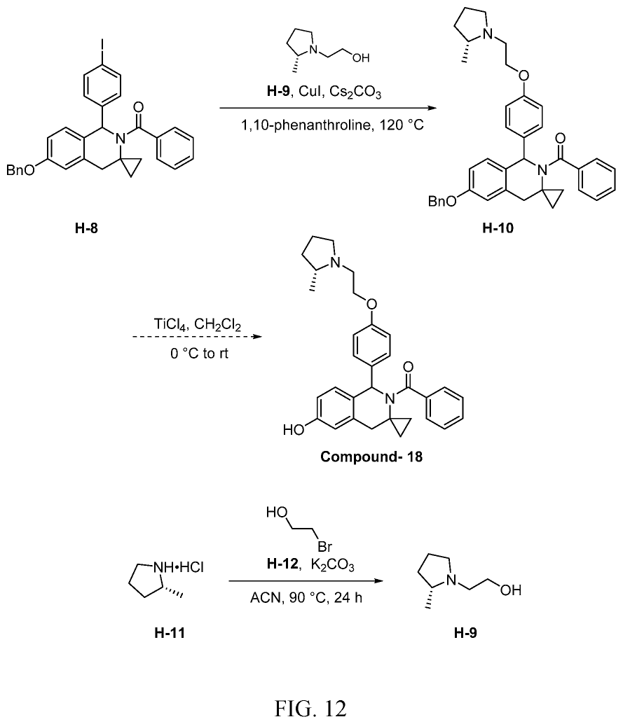

FIG. 12 is a chemical scheme of preparing compound 18 from H-8.

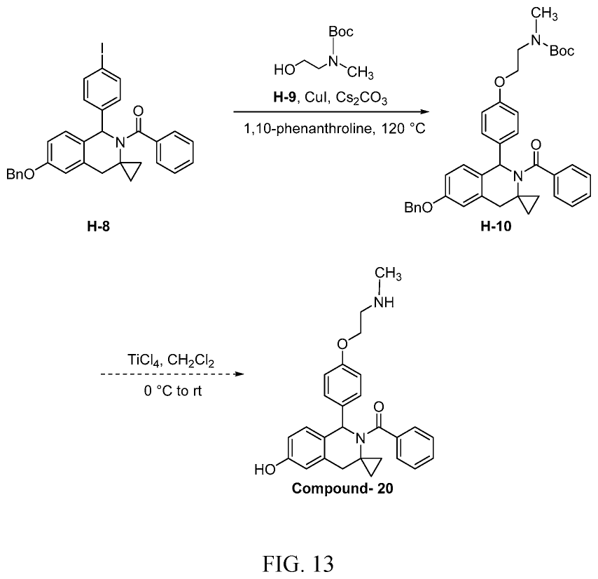

FIG. 13 is a chemical scheme of preparing compound 20 from H-8.

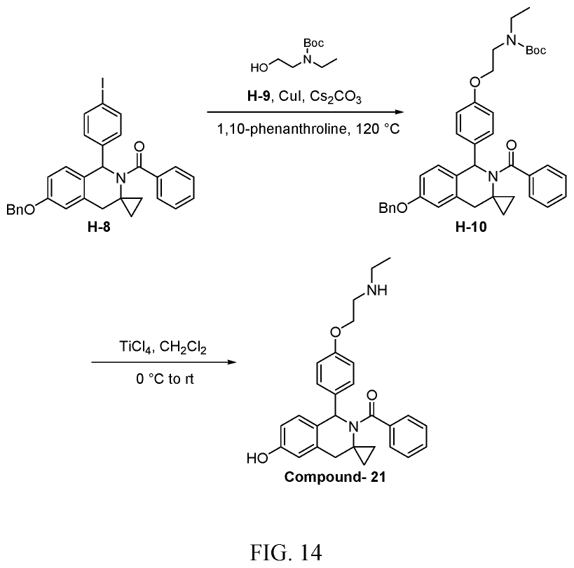

FIG. 14 is a chemical scheme of preparing compound 21 from H-8.

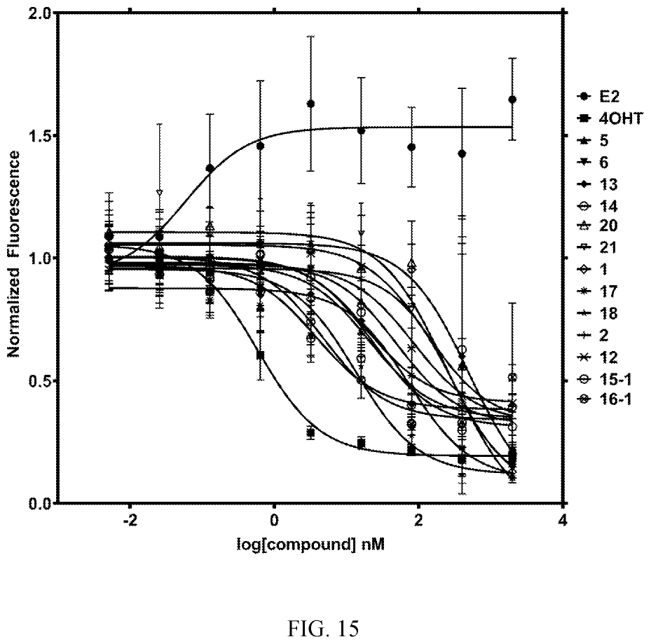

FIG. 15 is a graph of normalized fluorescence versus log[compound] (nM) of compounds of formula (I) compared to 17-beta-estradiol (E2) and 4-hydroxytamoxifen (4OHT). The tested compounds were E2 (●), 4OHT (▬), 5 (▴), 6 (▾), 13 (♦), 14 (◯), 20 (Δ), 21 (∇), 1 (⋄), 17 (*), 18 ( [symbol] ), 2 (|) 12 (33 ), 15 (⊙), and 16 (⊗).

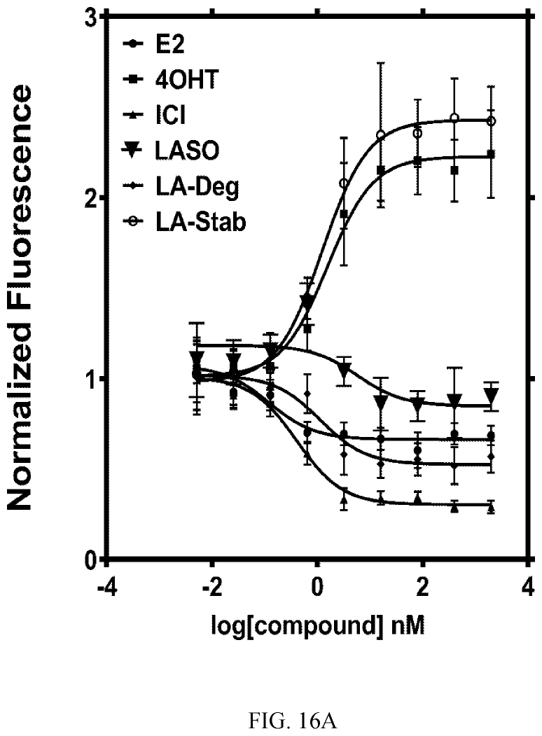

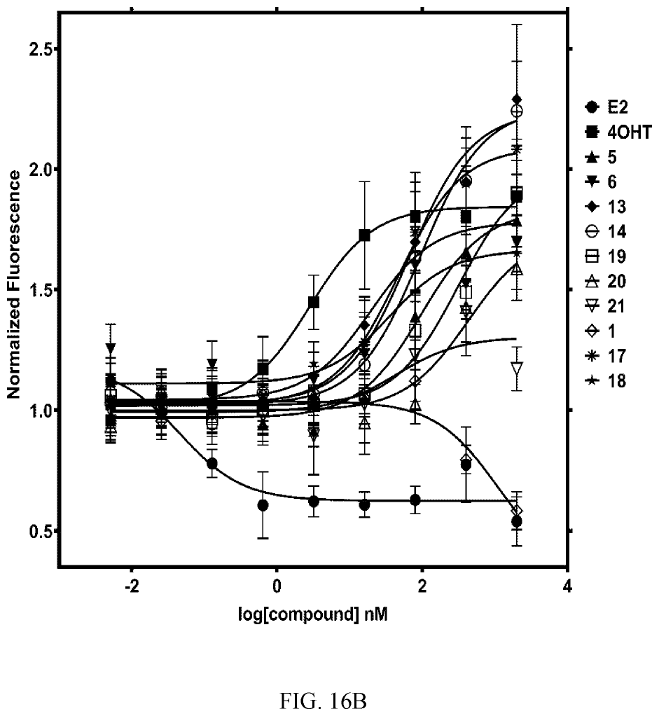

FIGS. 16A-16B are graphs of normalized fluorescence versus log[compound] (nM) of known antagonists (FIG. 16A) and compounds of formula (I) plus E2 and 4OHT (FIG. 16B).

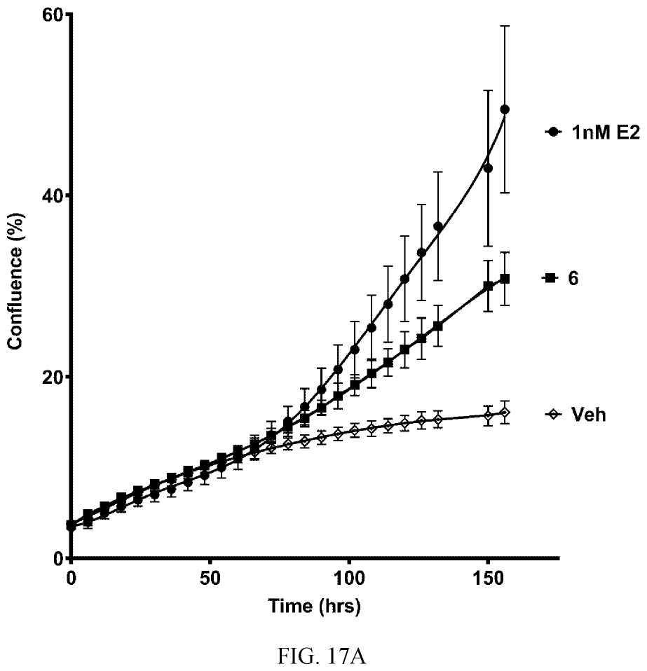

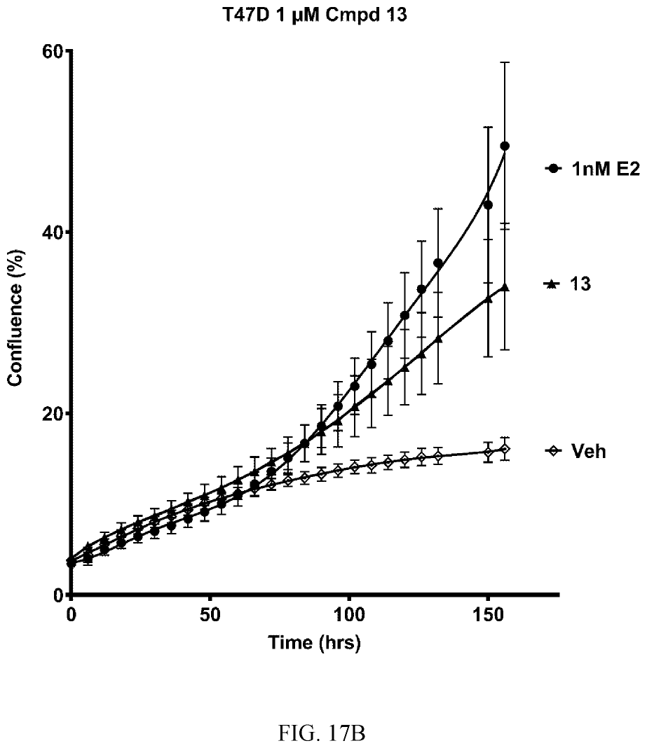

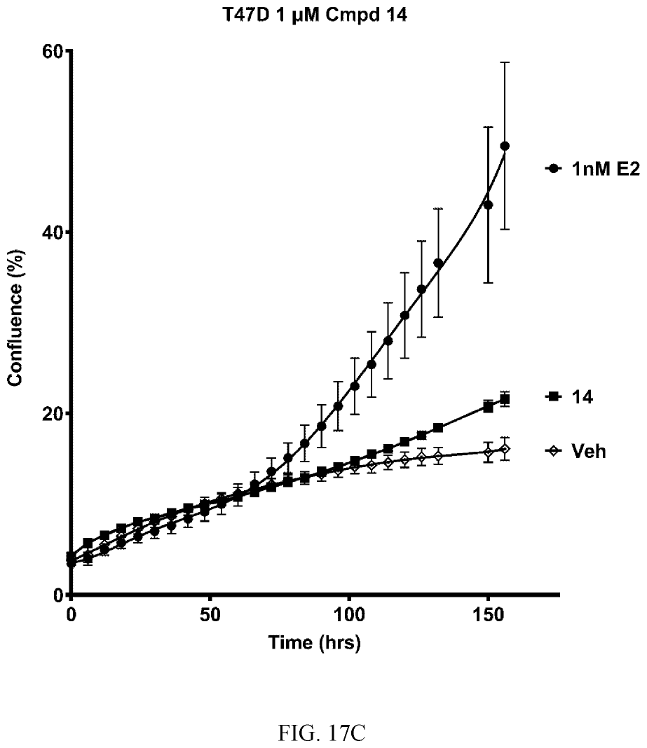

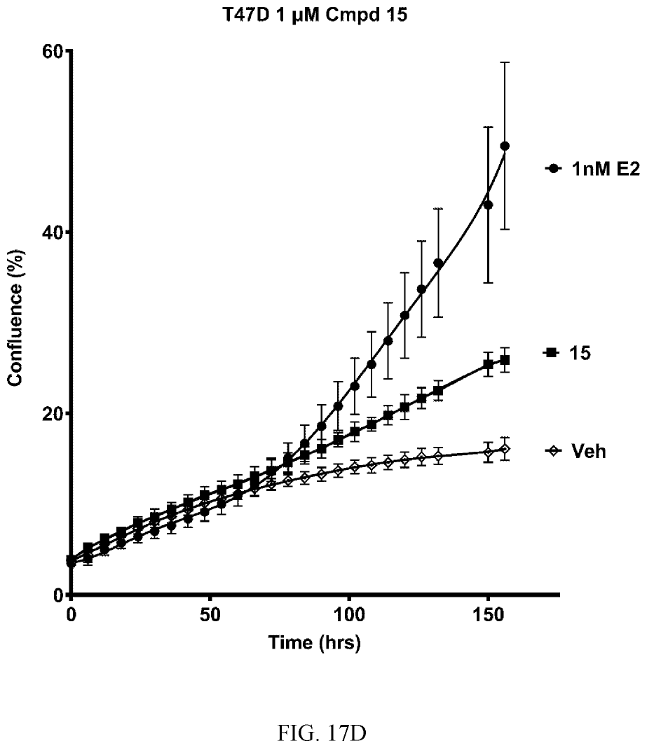

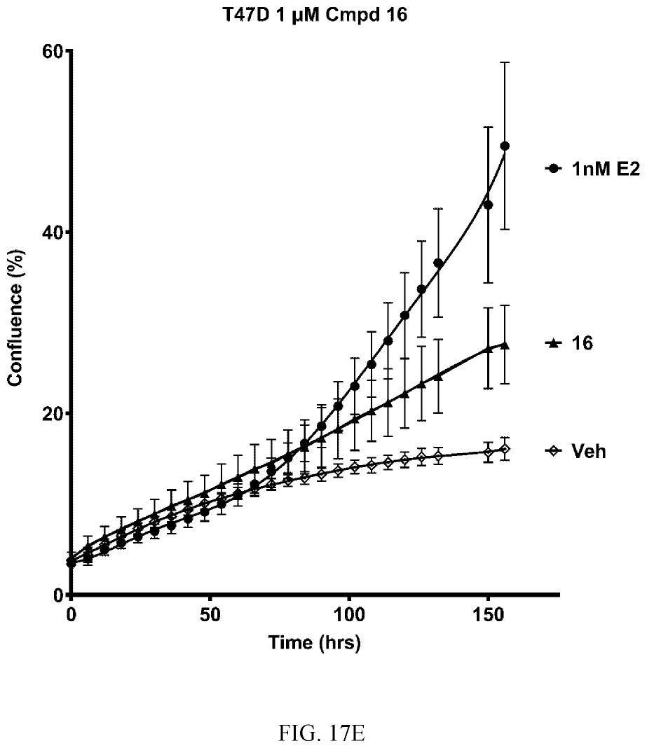

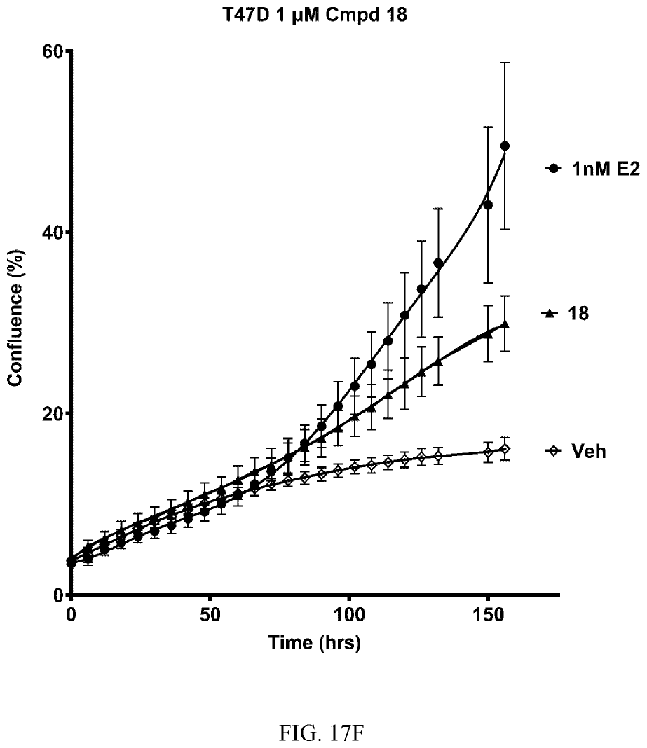

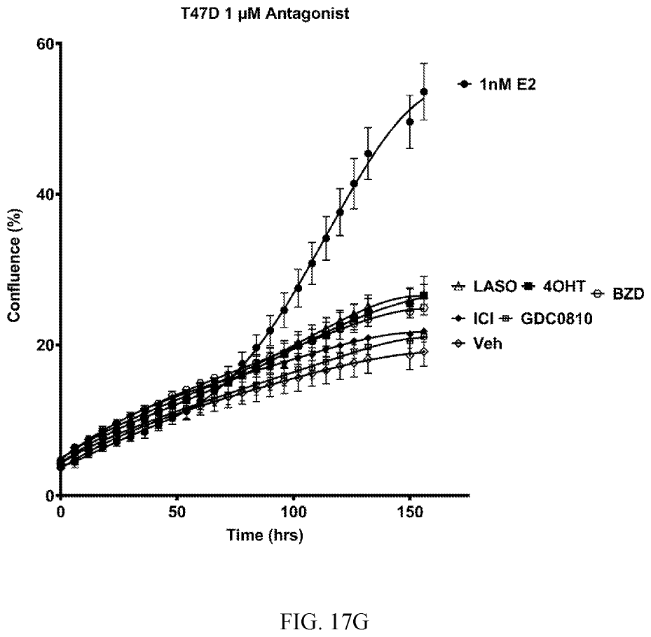

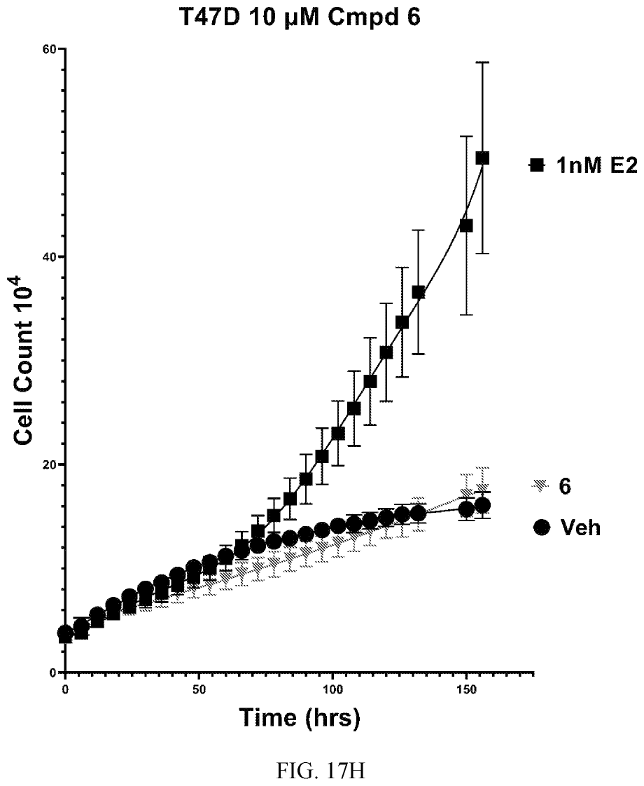

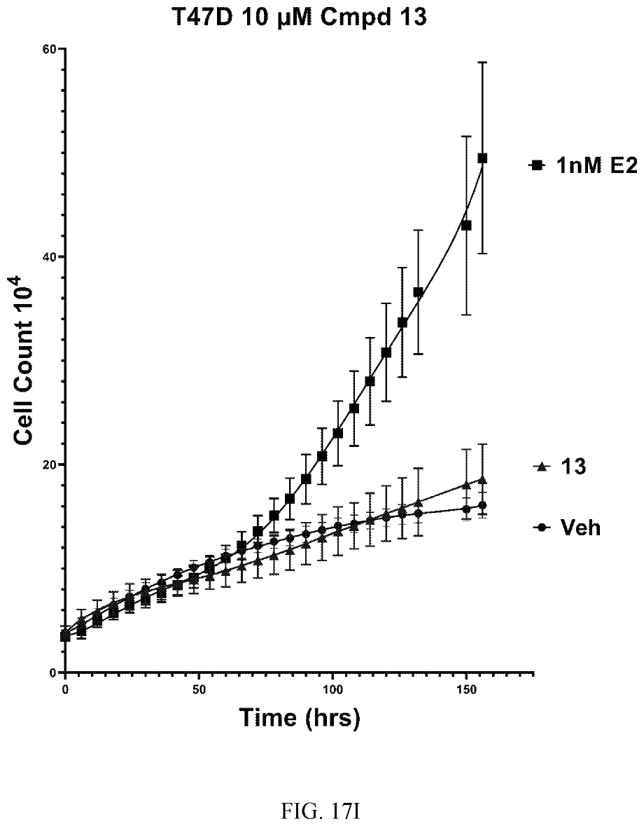

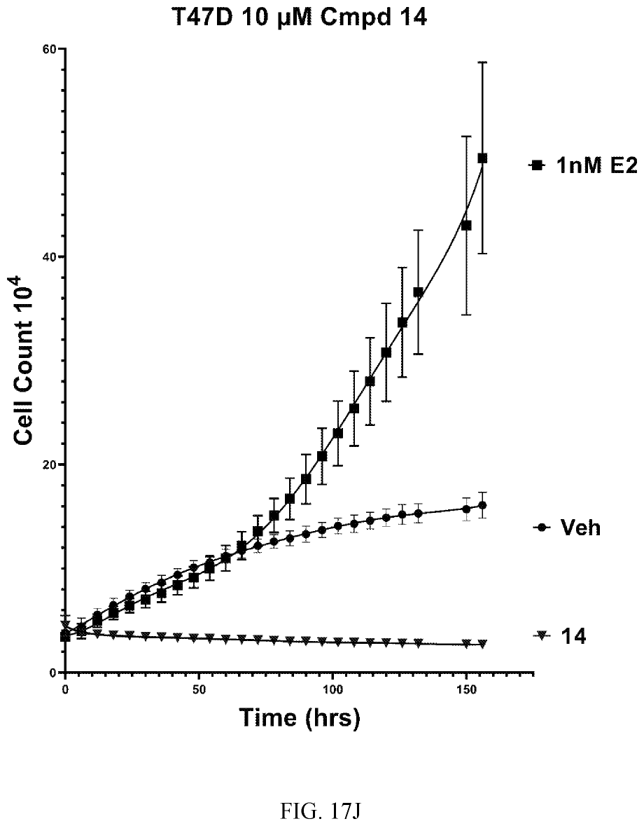

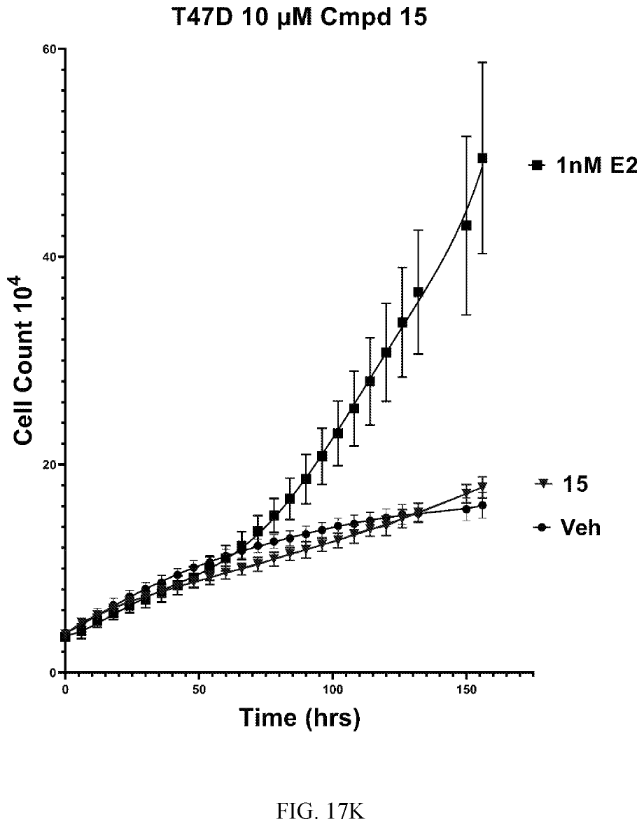

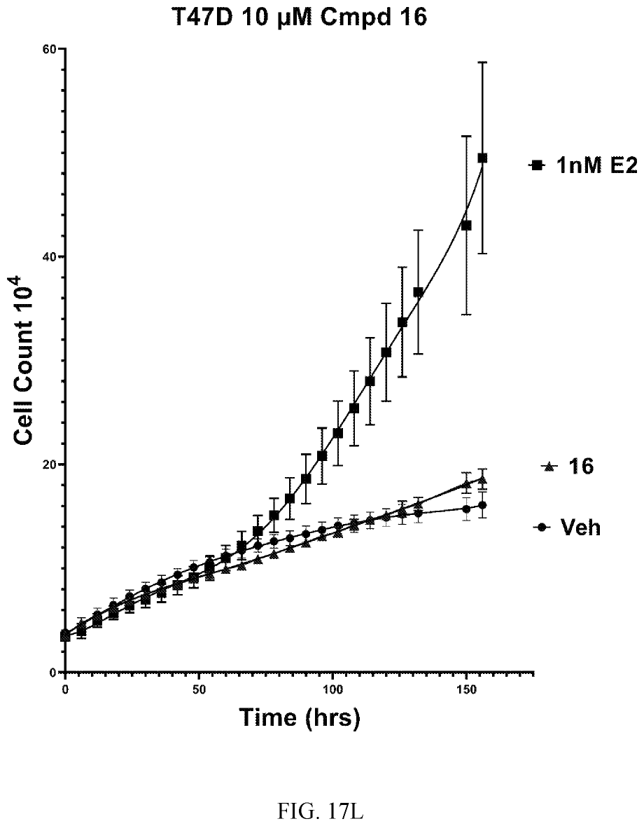

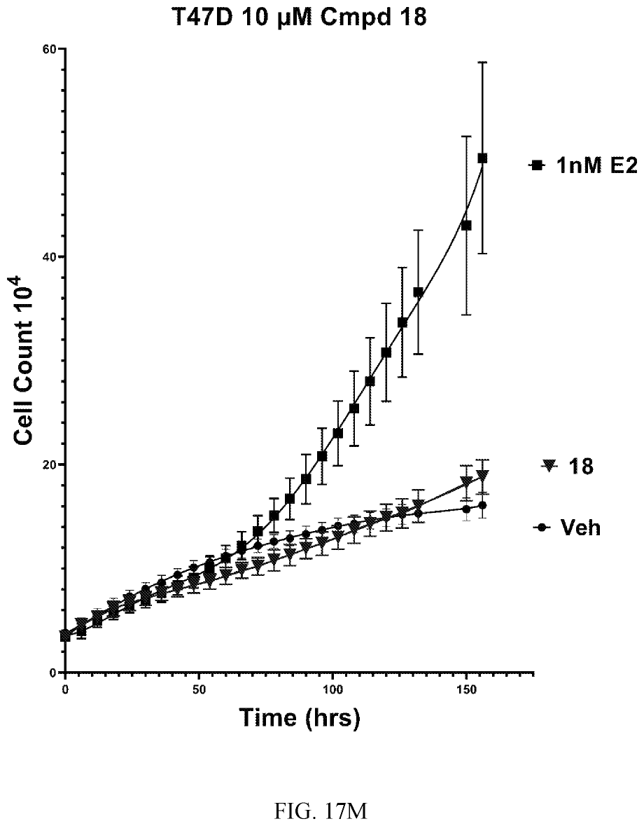

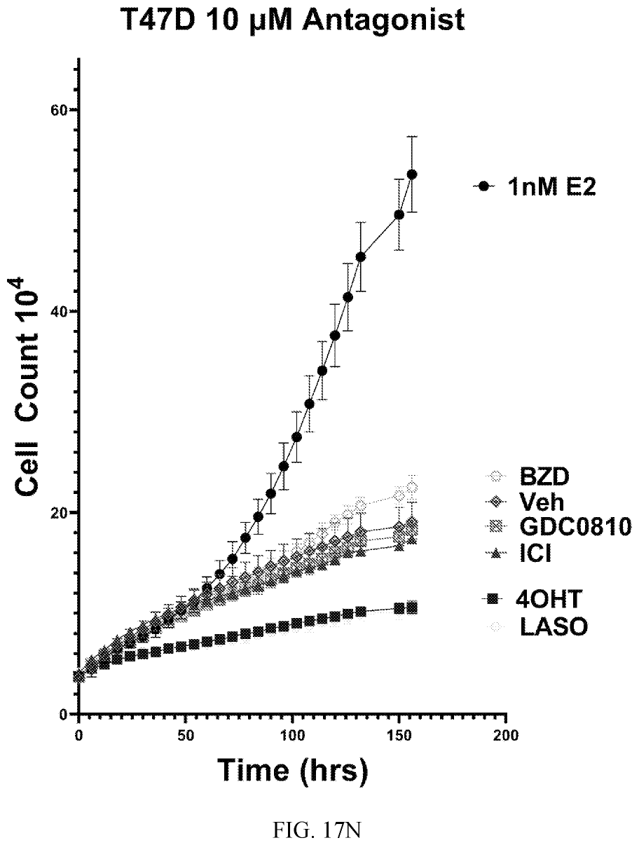

FIGS. 17A-17N are graphs of percent confluence versus time (hours) of cellular proliferation of T47D breast cancer cells of either 1 μM of the following: compound 6 (FIG. 17A), compound 13 (FIG. 17B), compound 14 (FIG. 17C), compound 15 (FIG. 17D), compound 16 (FIG. 17E), 18 (FIG. 17F), and known antagonists (FIG. 17G) or 10 μM of the following compounds: compound 6 (FIG. 17H), compound 13 (FIG. 17I), compound 14 (FIG. 17J), compound 15 (FIG. 17K), compound 16 (FIG. 17L), 18 (FIG. 17M), and known antagonists (FIG. 17N). Each of the compounds was tested against E2 and vehicle only.

FIG. 18 is a bar graph of signal strength of MCF7 cellular viability in a crystal violet endpoint assay using compounds of formula (I) with the bars representing left-to-right what is described in the legend top-to-bottom.

FIG. 19 is a bar graph of anti-proliferative activities in MCF7 breast cancer cells. Cells were treated for 150 hours in the presence of 1 nM estradiol (E2). Data were the mean of three replicates±standard deviation.

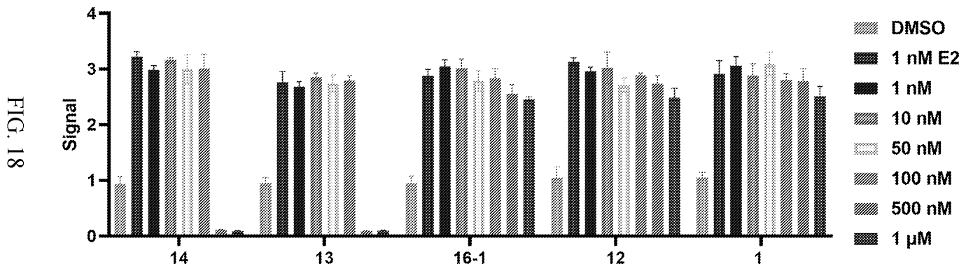

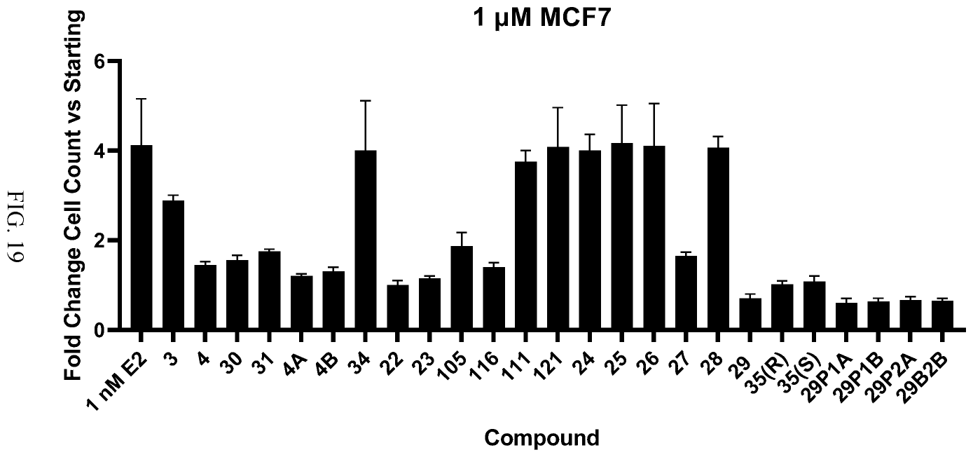

FIG. 20 is a bar graph of anti-proliferative activities in T47D breast cancer cells. Cells were treated for 150 hours in the presence of 1 nM estradiol (E2). Data were the mean of three replicates±standard deviation.

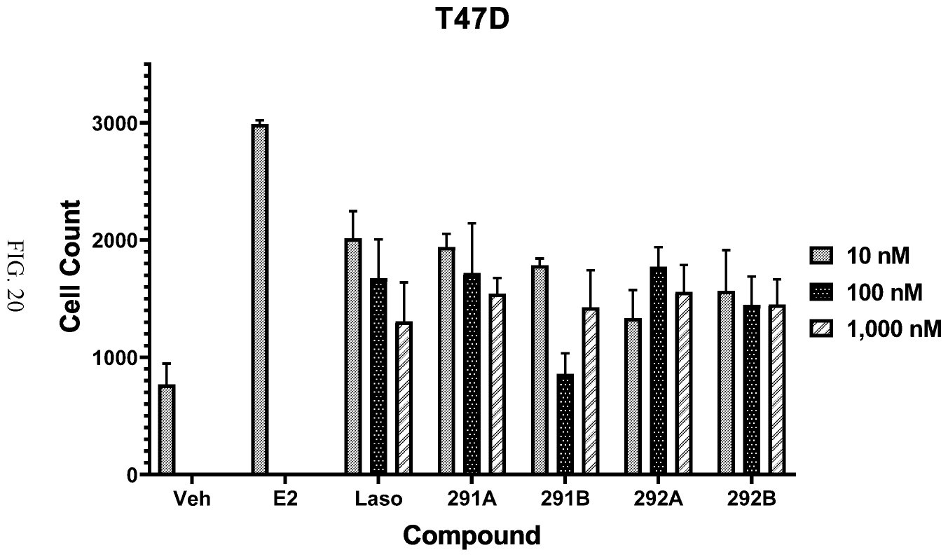

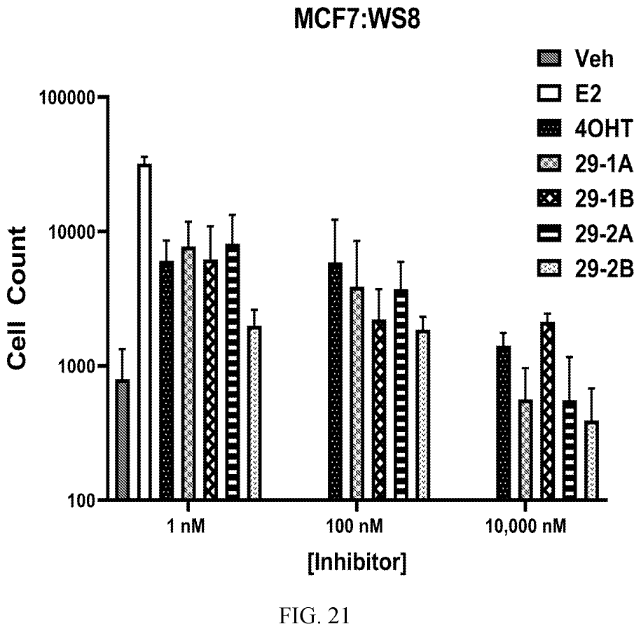

FIG. 21 is a bar graph of anti-proliferative activities in MCF7:WS8 breast cancer cells. Cells were treated for 150 hours in the presence of 1 nM estradiol (E2). Data were the mean of three replicates±standard deviation. The bars represent from left-to-right: vehicle, E2, and then the five tested compounds, as described in the legend top-to-bottom, for three concentrations.

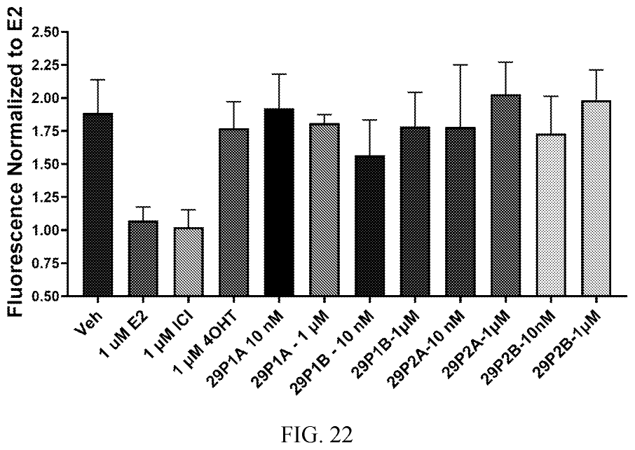

FIG. 22 is a bar graph of in-cell western analysis to measure endogenous ERa in MCF7 breast cancer cells. Cells were treated for 24 hours. Data were the mean of three replicates±standard deviation after normalization to cell count per-well.

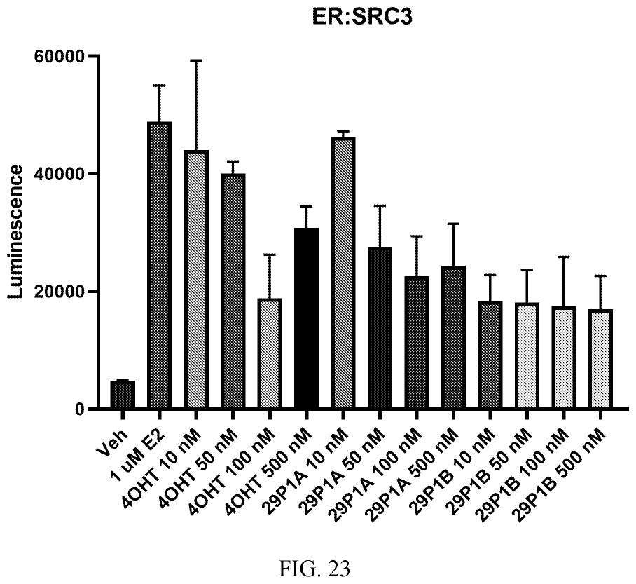

FIG. 23 is a bar graph of NANOBIT™ split luciferase assay (Promega, Madison, WI) of ERα-SRC3 binding in HEK293T cells. Cells were treated for 24 hours. Data were the mean of three replicates±standard deviation after normalization to cell count per-well.

### DETAILED DESCRIPTION OF THE INVENTION

Competitive antiestrogens act by binding to the estrogen receptor alpha and eliciting a conformational change that prevents the formation of the functional transcriptional complexes. Therapeutically important mechanisms of action include inhibition of ERα transcriptional activity, impact on receptor nuclear lifetime (which correlates with side effect profiles), and inhibition of cancer cellular proliferation, tumor growth, and metastasis.

In an embodiment, the invention provides a compound of formula (I) or a pharmaceutically acceptable salt thereof:

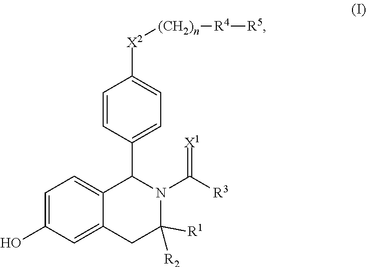

wherein

X¹ is O or S;

X² is a bond, alkenyl, O, S, or NR₆;

R¹ is selected from H and C₁₋₆ alkyl;

R² is C₁₋₆ alkyl; or

R¹ and R² together form C₃₋₆ cycloalkyl,

R³ is C₂₋₁₂ alkyl or phenyl optionally substituted with at least one substituent selected from C₁₋₆ alkyl, C₃₋₆ cycloalkyl, hydroxy, alkoxy, cycloalkoxy, halo, and amino; or

R³ is phenyl with two substituents that together with the phenyl form an optionally substituted bicyclic nitrogen-containing heteroaryl;

R⁴ is a nitrogen-containing C₃₋₇ heterocycloalkyl, —NR⁷—, or —C(═O)O—;

R⁵, R⁶, and R⁷ are the same or different and each is a hydrogen or C₁₋₆ alkyl; and

n is 0 or an integer of 1 to 5,

wherein the C₁₋₆ alkyl and C₃₋₆ cycloalkyl can be substituted with one or more substituents selected from hydroxy, halo, alkoxy, alkyl, cycloalkyl, heterocycloalkyl, aryl, heteroaryl, amino, alkylamino, dialkylamino, and carboxylato.

In some embodiments of formula (I) or a pharmaceutically acceptable salt thereof, X¹ and X² are both O. In other embodiments of formula (I) or a pharmaceutically acceptable salt thereof, X¹ is O and either (i) X² is a bond, (ii) X² is S, or (iii) X² is NR⁶. In yet other embodiments of formula (I) or a pharmaceutically acceptable salt thereof, X¹ is O and X² is alkenyl (e.g., vinyl).

In some embodiments of formula (I) or a pharmaceutically acceptable salt thereof, (i) R¹ and R² are both C₁₋₆ alkyl (e.g., methyl), (ii) R¹ is H and R² is C₁₋₆ alkyl (e.g., methyl), or (iii) R¹ and R² together form C₃₋₆ cycloalkyl (i.e., cyclopropyl, cyclobutyl, cyclopentyl, or cyclohexyl). In a preferred embodiment of formula (I), R¹ and R² together form cyclopropyl.

In some embodiments of formula (I) or a pharmaceutically acceptable salt thereof, R³ is C₂₋₁₂ alkyl (e.g., ethyl, n-propyl, isopropyl, n-butyl, isobutyl, sec-butyl, n-pentyl, isopentyl, neopentyl, n-hexyl, isohexyl, 3-methylpentyl, 2,2-dimethylbutyl, or 2,3-dimethylbutly, etc.). In preferred embodiments of formula (I), R³ is neopentyl.

In some embodiments of formula (I) or a pharmaceutically acceptable salt thereof, R³ is phenyl, in which the phenyl is optionally substituted with at least one substituent selected from C₁₆ alkyl, C₃₋₆ cycloalkyl, hydroxy, alkoxy, cycloalkoxy, halo, and amino. In a preferred aspect of this embodiment, the phenyl is substituted in at least a para position (e.g., one substituent in a para position) relative to the rest of the molecule. In preferred embodiments, R³ is unsubstituted phenyl or phenyl substituted at the 3- or 4-position with C₁₋₆ alkyl, haloalkyl, or halo. More preferably, R³ is phenyl substituted at the 3-position with C₁₋₆ alkyl, haloalkyl, or halo. Examples of suitable substituents for phenyl include 3-methyl, 4-methyl, 3-trifluoromethyl, 4-trifluoromethyl, 3-chloro, 4-chloro, 3-bromo, and 4-bromo.

In some embodiments of formula (I) or a pharmaceutically acceptable salt thereof, R³ is substituted phenyl, in which two substituents together with the phenyl group form an optionally substituted bicyclic nitrogen-containing heteroaryl. The optionally substituted bicyclic nitrogen-containing heteroaryl contains the phenyl group fused to a 5- or 6-membered nitrogen-containing heteroaryl that has 1 or 2 N atoms (e.g., pyrazolyl, pyrrolyl, pyridinyl, pyridazinyl, pyrimidinyl, or pyrazinyl). The bicyclic nitrogen-containing heteroaryl can be, for example,

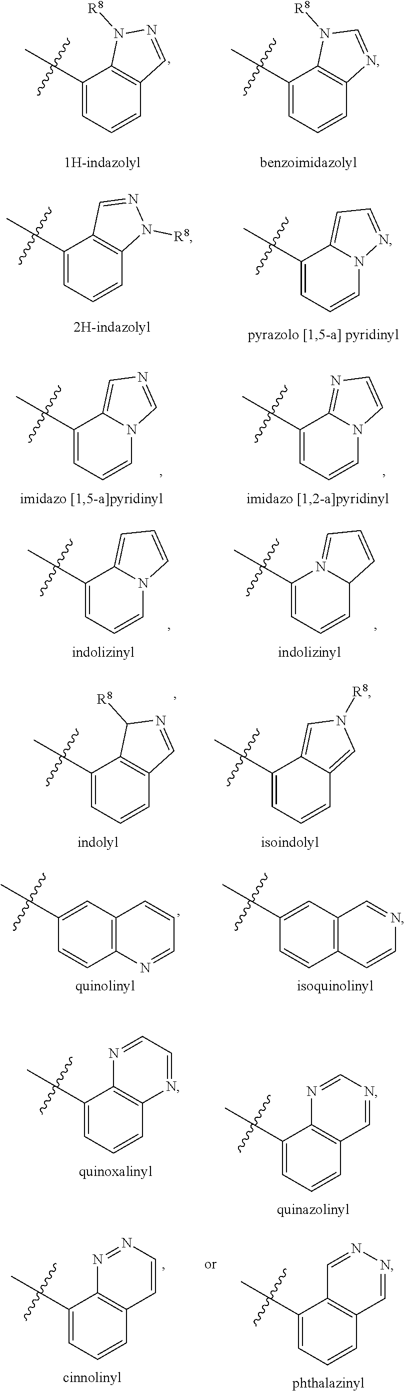

in which R⁸ is hydrogen or C₁₋₆ alkyl, and the bicyclic ring is further optionally substituted (e.g., C₁₋₆ alkyl, hydroxy, haloalkyl, and/or halo).

In preferred embodiments, the optionally substituted bicyclic nitrogen-containing heteroaryl is indazolyl (1H or 2H), indolizinyl, pyrazolo[1,5-a]pyridinyl, or imidazo[1,5-a]pyridinyl, each of which is optionally substituted. For example, the nitrogen in the nitrogen-containing heteroaryl (R⁸ when present) can be substituted with C₁₋₆ alkyl. Alternatively, or in addition, other positions around the ring of the bicyclic nitrogen-containing heteroaryl can include one or more substituents, as described herein (e.g., C₁₋₆ alkyl, hydroxy, haloalkyl, or halo).

In some embodiments of formula (I) or a pharmaceutically acceptable salt thereof, R⁴ is a nitrogen-containing C₃₋₇ heterocycloalkyl. In certain preferred embodiments, R⁴ is 3-azetidinyl, 1-pyrrolidinyl, 3-pyrrolidinyl, 1-piperidinyl, 4-piperidinyl, 1-piperazinyl, or 1-azepanyl, particularly, 1-pyrrolidinyl or 3-pyrrolidinyl.

In other embodiments of formula (I) or a pharmaceutically acceptable salt thereof, R⁴ is —NH— or —N(C₁₋₃ alkyl)—.

In some embodiments of formula (I) or a pharmaceutically acceptable salt thereof, R⁵ is C₁₋₃ alkyl (i.e., methyl, ethyl, n-propyl, or isopropyl). In some aspects of this embodiment, the C₁₋₃ alkyl of R⁵ is substituted with halo, such as a terminal fluoro or chloro.

In some embodiments of formula (I) or a pharmaceutically acceptable salt thereof, n is 0, 1, or 2. Preferably, n is 0 or 2.

In some preferred embodiments of formula (I) or a pharmaceutically acceptable salt thereof, X¹ is O, X² is O or S, R¹ and R² together form cyclopropyl, R³ is phenyl substituted at the 3-position with a substituent selected from C₁₋₆ alkyl, C₃₋₆ cycloalkyl, hydroxy, alkoxy, cycloalkoxy, halo, and amino, R⁴ is a nitrogen-containing C₄₋₅ heterocycloalkyl, R₅ is optionally substituted C₁₋₆ alkyl, and n is 1 or 2.

In some aspects, the compound of formula (I) has a core structure of formula (Ia)

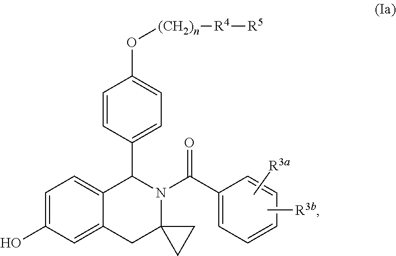

wherein

R^{3a} and R^{3b} are the same or different and each is selected from H, C₁₋₆ alkyl, C₃₋₆ cycloalkyl, hydroxy, alkoxy, cycloalkoxy, halo, and amino; or

R^{3a} and R^{3b} together with the phenyl group form an optionally substituted bicyclic nitrogen-containing heteroaryl;

R⁴ is a nitrogen-containing C₃₋₇ heterocycloalkyl, or —NR⁷—;

R⁵ and R⁷ are the same or different and each is a hydrogen or C₁₋₆ alkyl; and

n is 0 or an integer of 1 to 5,

wherein the C₁₋₆ alkyl and C₃₋₆ cycloalkyl can be substituted with one or more substituents selected from hydroxy, halo, alkoxy, alkyl, cycloalkyl, heterocycloalkyl, aryl, heteroaryl, amino, alkylamino, dialkylamino, and carboxylato.

In some embodiments of formula (Ia) or a pharmaceutically acceptable salt thereof, R^{3a} and R^{3b} are each hydrogen or one of R^{3a} and R^{3b} is hydrogen and the other is C₁₋₆ alkyl, haloalkyl, or halo. The substituted phenyl includes, for example, 3-methyl, 4-methyl, 3-trifluoromethyl, 4-trifluoromethyl, 3-chloro, 4-chloro, 3-bromo, and 4-bromo.

In some embodiments of formula (Ia) or a pharmaceutically acceptable salt thereof, R^{3a} and R^{3b} together with the phenyl group form an optionally substituted bicyclic nitrogen-containing heteroaryl. The optionally substituted bicyclic nitrogen-containing heteroaryl contains the phenyl group fused to a 5- or 6-membered nitrogen-containing heteroaryl that has 1 or 2 N atoms (e.g., pyrazolyl, pyrrolyl, pyridinyl, pyridazinyl, pyrimidinyl, or pyrazinyl). The bicyclic nitrogen-containing heteroaryl can be, for example,

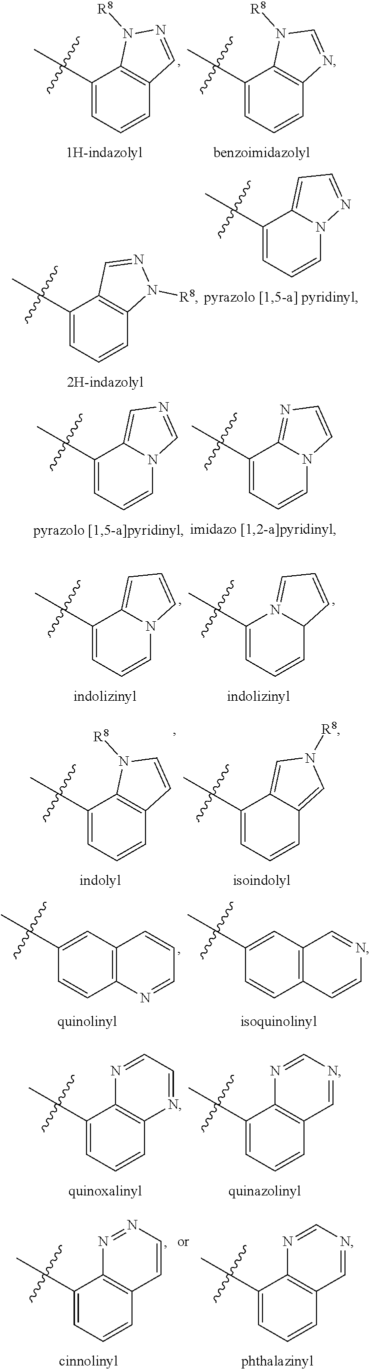

in which R⁸ is hydrogen or C₁₋₆ alkyl, and the bicyclic ring is further optionally substituted (e.g., C₁₋₆ alkyl, hydroxy, haloalkyl, and/or halo).

In preferred embodiments, the optionally substituted bicyclic nitrogen-containing heteroaryl is indazolyl (1H or 2H), indolizinyl, pyrazolo[1,5-a]pyridinyl, or imidazo[1,5-a]pyridinyl, each of which is optionally substituted. For example, the nitrogen in the nitrogen-containing heteroaryl (R⁸ when present) can be substituted with C₁₋₆ alkyl. Alternatively, or in addition, other positions around the ring of the bicyclic nitrogen-containing heteroaryl can include one or more substituents, as described herein (e.g., C₁₋₆ alkyl, hydroxy, haloalkyl, or halo).

In some embodiments of formula (Ia) or a pharmaceutically acceptable salt thereof, R⁴ is a nitrogen-containing C₃₋₇ heterocycloalkyl. In certain preferred embodiments, R⁴ is 3-azetidinyl, 1-pyrrolidinyl, 3-pyrrolidinyl, 1-piperidinyl, 4-piperidinyl, 1-piperazinyl, or 1-azepanyl, particularly, 1-pyrrolidinyl or 3-pyrrolidinyl.

In other embodiments of formula (Ia) or a pharmaceutically acceptable salt thereof, R⁴ is —NH— or —N(C₁₋₃ alkyl)—.

In some embodiments of formula (Ia) or a pharmaceutically acceptable salt thereof, R⁵ is C₁₋₃ alkyl (i.e., methyl, ethyl, n-propyl, or isopropyl).

In some embodiments of formula (Ia) or a pharmaceutically acceptable salt thereof, n is 0, 1, or 2. Preferably, n is 0 or 2.

The compound of formula (I), including the compound of formula (Ia), can have any suitable stereochemistry and can be in the form of a single stereoisomer or a mixture of two or more stereoisomers (e.g., an epimer, a mixture of diastereomers and/or enantiomers, a racemic mixture). In some embodiments of formula (I) and (Ia) or a pharmaceutically acceptable salt thereof, the compound is the R-enantiomer. In other embodiments, the compound is the S-enantiomer. In other embodiments, the compound exists as a racemic mixture.

In an embodiment, the present invention provides exemplary compounds of formula (I) and (Ia), including

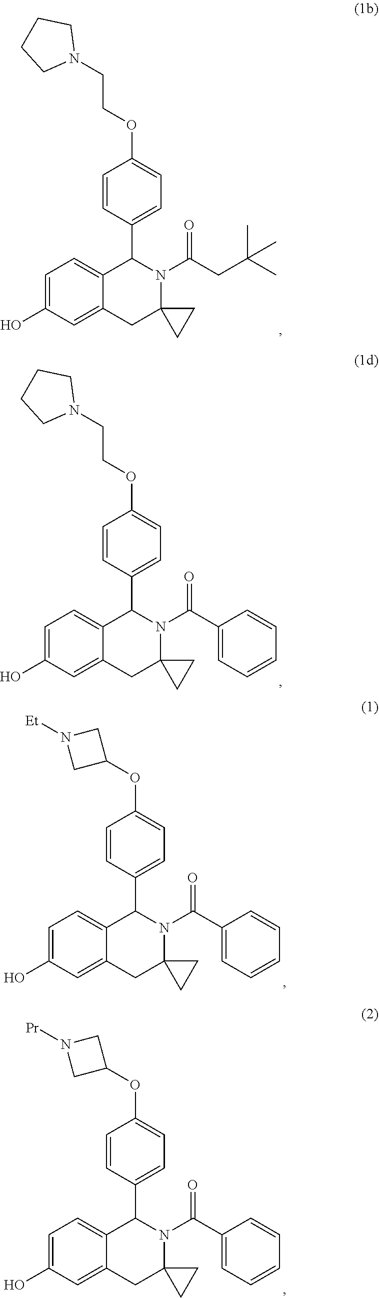

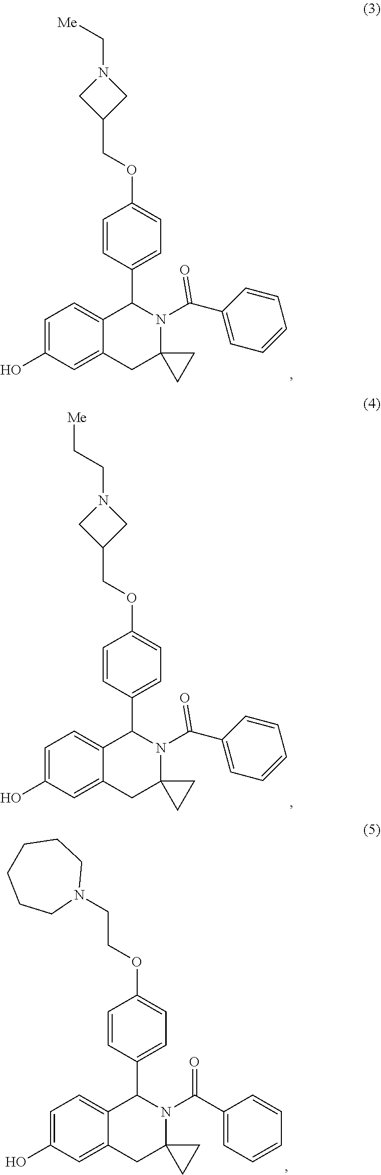

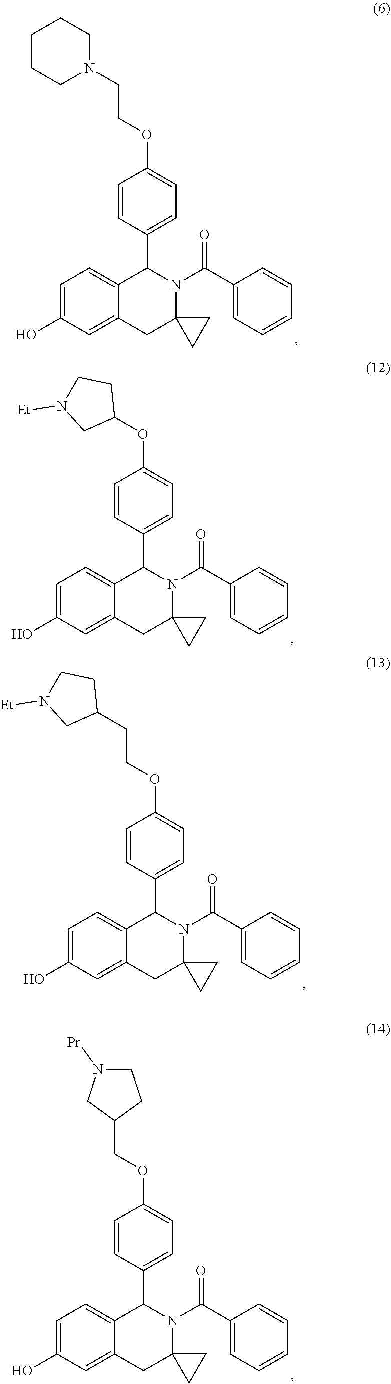

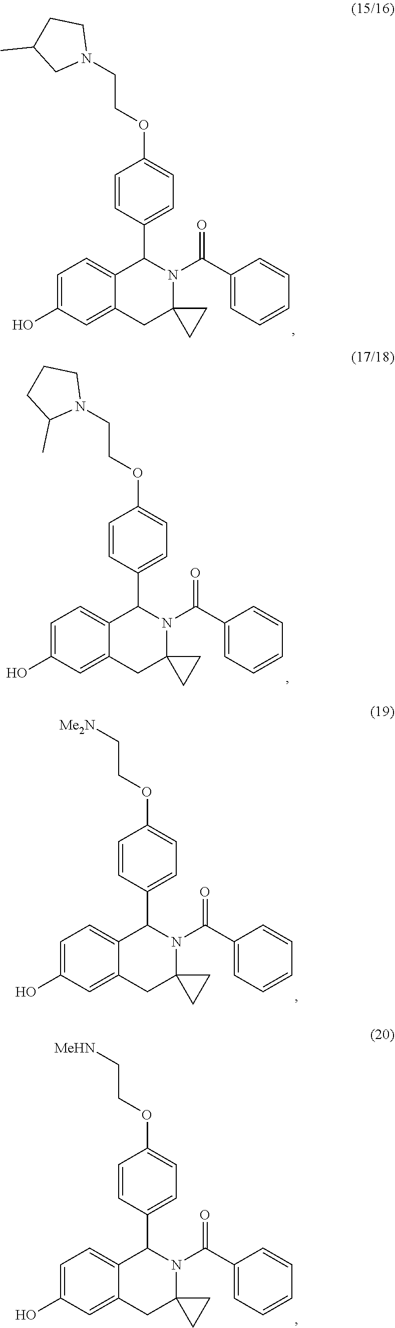

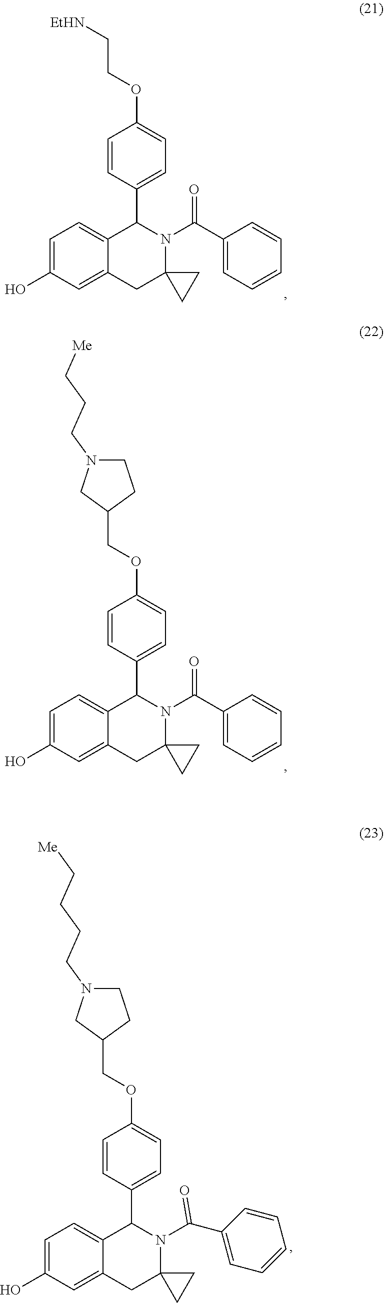

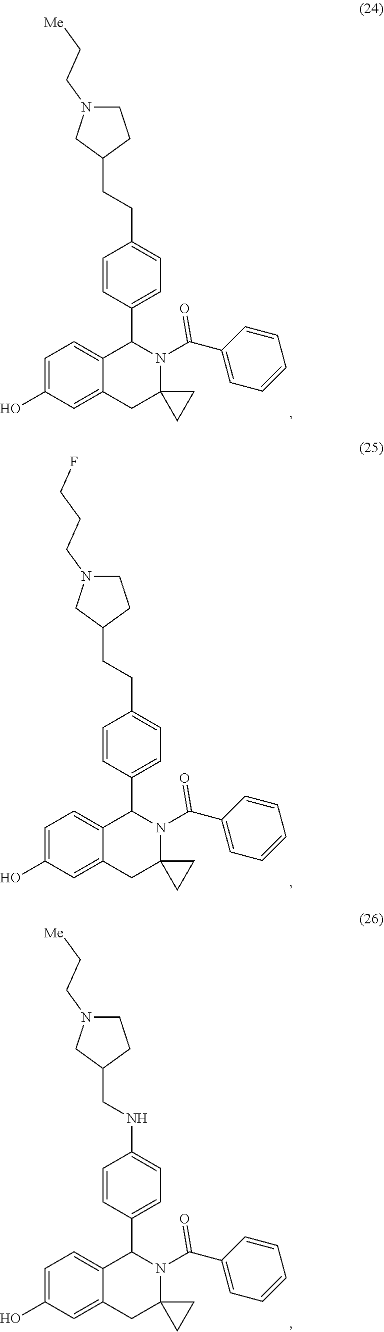 [SMILES: CCCN1CCC(CCc2ccc(C3c4ccc(O)cc4CC4(CC4)N3C(=O)c3ccccc3)cc2)C1 ; O=C(c1ccccc1)N1C(c2ccc(CCC3CCN(CCCF)C3)cc2)c2ccc(O)cc2CC12CC2 ; CCCN1CCC(CNc2ccc(C3c4ccc(O)cc4CC4(CC4)N3C(=O)c3ccccc3)cc2)C1]

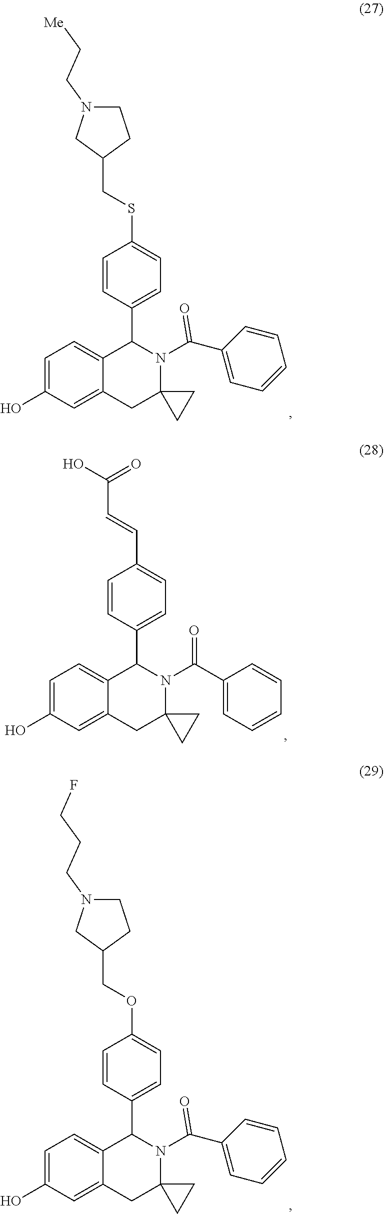 [SMILES: CCCN1CCC(CSc2ccc(C3c4ccc(O)cc4CC4(CC4)N3C(=O)c3ccccc3)cc2)C1 ; O=C(O)/C=C/c1ccc(C2c3ccc(O)cc3CC3(CC3)N2C(=O)c2ccccc2)cc1 ; O=C(c1ccccc1)N1C(c2ccc(OCC3CCN(CCCF)C3)cc2)c2ccc(O)cc2CC12CC2]

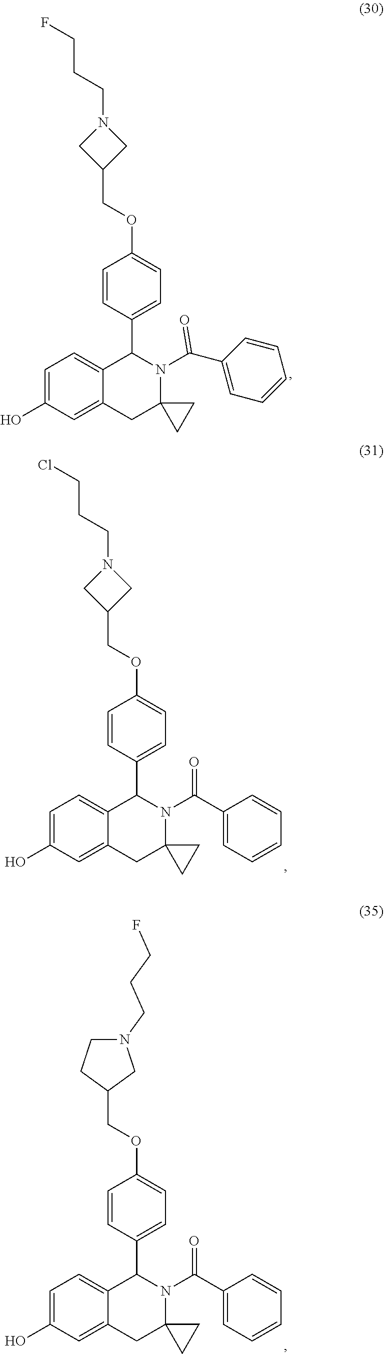 [SMILES: O=C(c1ccccc1)N1C(c2ccc(OCC3CN(CCCF)C3)cc2)c2ccc(O)cc2CC12CC2 ; O=C(c1ccccc1)N1C(c2ccc(OCC3CN(CCCCl)C3)cc2)c2ccc(O)cc2CC12CC2 ; O=C(c1ccccc1)N1C(c2ccc(OCC3CCN(CCCF)C3)cc2)c2ccc(O)cc2CC12CC2]

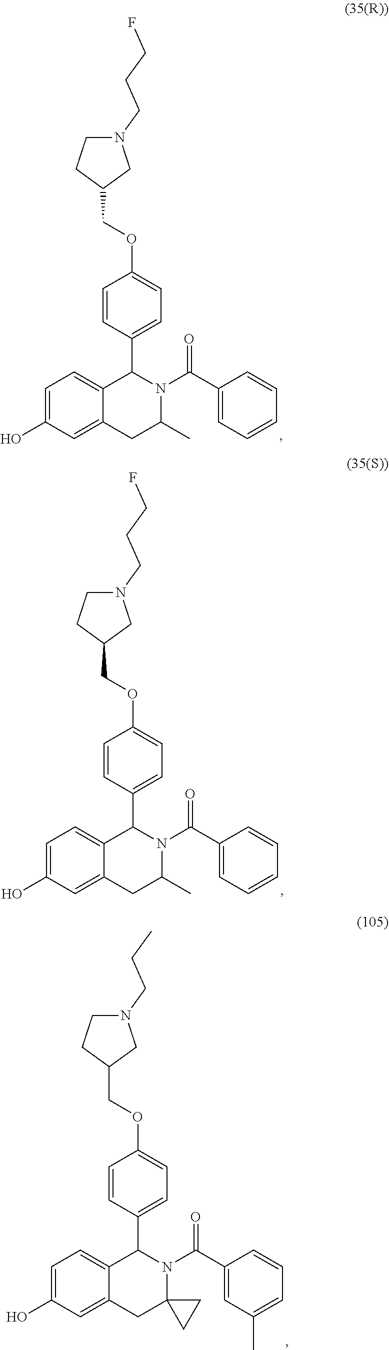

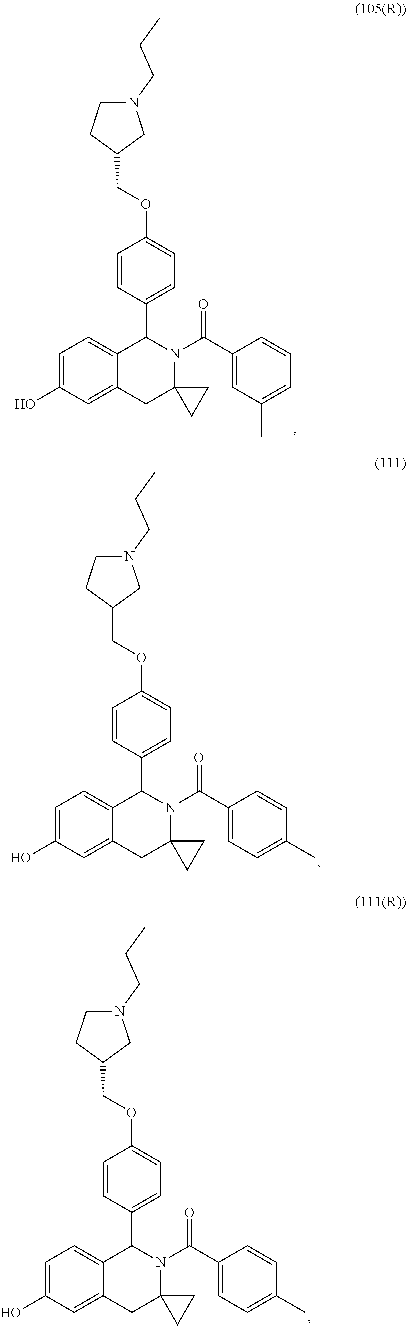

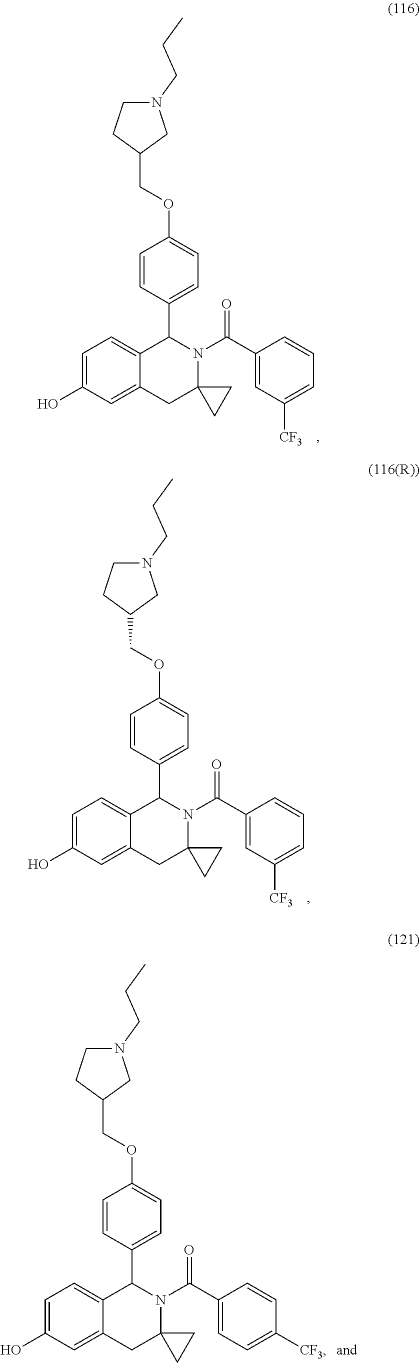

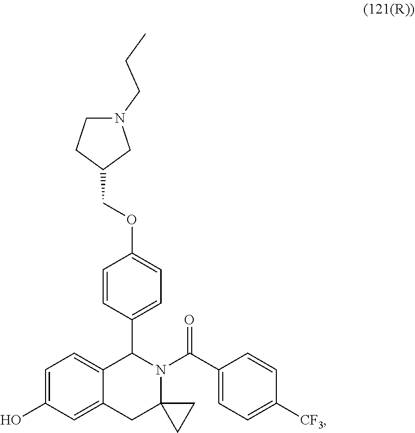

or a stereoisomer thereof and/or a pharmaceutically acceptable salt thereof. Compounds with one number (e.g., 1, 2, 13, 14, etc.) represent a mixture of stereoisomers, whereas compounds with two numbers (e.g., 15/16, 17/18) represent the different stereoisomers of substituent R⁵.

Particularly preferred compounds of formulas (I) and (Ia) include

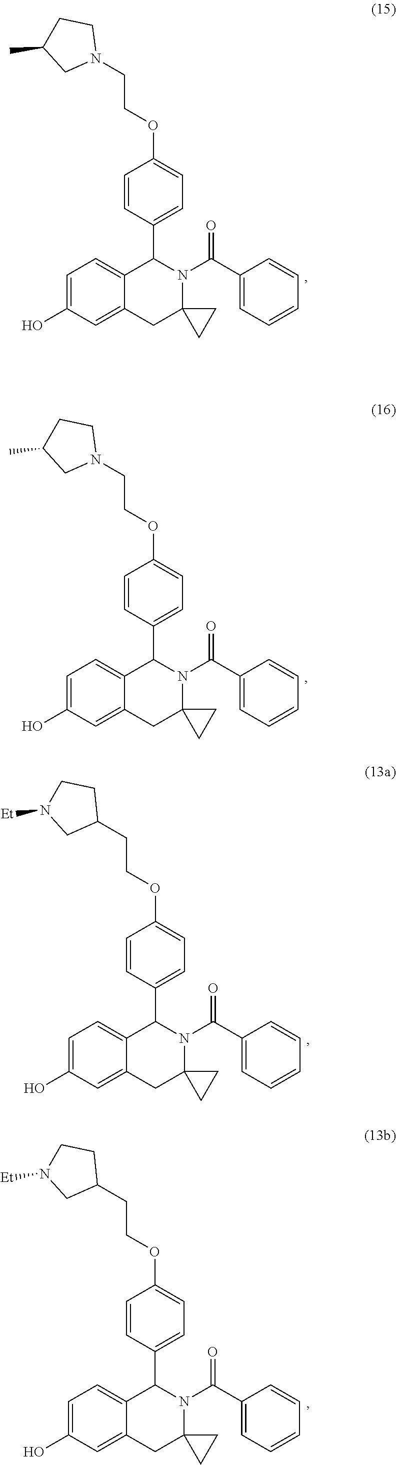

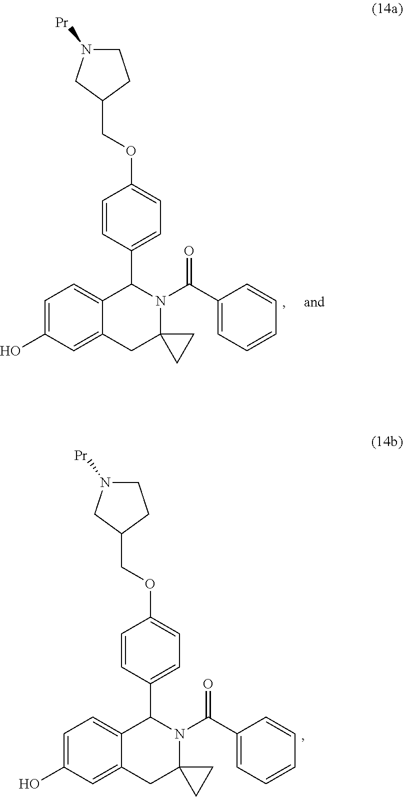

or a pharmaceutically acceptable salt thereof

In any of the embodiments above, the term “alkyl” implies a straight-chain or branched alkyl substituent containing from, for example, from about 1 to about 6 carbon atoms, e.g., from about 1 to about 4 carbon atoms. Examples of alkyl group include methyl, ethyl, n-propyl, isopropyl, n-butyl, sec-butyl, isobutyl, tert-butyl, n-pentyl, isopentyl, n-hexyl, and the like. This definition also applies wherever “alkyl” occurs as part of a group, such as, e.g., in C₃₋₆ cycloalkylalkyl, hydroxyalkyl, haloalkyl (e.g., monohaloalkyl, dihaloalkyl, and trihaloalkyl), aminoalkyl, alkylamino, dialkylamino, alkylaminoalkyl, dialkylaminoalkyl, arylcarbonylalkyl (-(alkyl)C(O)aryl), arylalkyl, etc. The alkyl can be substituted or unsubstituted, as described herein. Even in instances in which the alkyl is an alkylene chain (e.g., —(CH₂)ₙ—), the alkyl group can be substituted or unsubstituted.

In any of the embodiments above, the term “cycloalkyl,” as used herein, means a cyclic alkyl moiety containing from, for example, 3 to 6 carbon atoms. Examples of such moieties include cyclopropyl, cyclobutyl, cyclopentyl, cyclohexyl, and the like. The cycloalkyl can be substituted or unsubstituted, as described herein.

In any of the embodiments above, the term “aryl” refers to a mono, bi, or tricyclic carbocyclic ring system having one, two, or three aromatic rings, for example, phenyl, naphthyl, anthracenyl, or biphenyl. The term “aryl” refers to an unsubstituted or substituted aromatic carbocyclic moiety, as commonly understood in the art, and includes monocyclic and polycyclic aromatics such as, for example, phenyl, biphenyl, naphthyl, anthracenyl, pyrenyl, and the like. An aryl moiety generally contains from, for example, 6 to 30 carbon atoms, from 6 to 18 carbon atoms, from 6 to 14 carbon atoms, or from 6 to 10 carbon atoms. It is understood that the term aryl includes carbocyclic moieties that are planar and comprise 4n+2π electrons, according to Hickeys Rule, wherein n=1, 2, or 3. This definition also applies wherever “aryl” occurs as part of a group, such as, e.g., in haloaryl (e.g., monohaloaryl, dihaloaryl, and trihaloaryl), arylalkyl, etc. The aryl can be substituted or unsubstituted, as described herein.

In any of the embodiments above, the term “heteroaryl” refers to aromatic 5 or 6 membered monocyclic groups, 9 or 10 membered bicyclic groups, and 11 to 14 membered tricyclic groups which have at least one heteroatom (O, S, or N) in at least one of the rings. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic and tricyclic groups may contain only carbon atoms and may be saturated, partially saturated, or unsaturated. The nitrogen and sulfur atoms may optionally be oxidized, and the nitrogen atoms may optionally be quaternized. Heteroaryl groups which are bicyclic or tricyclic must include at least one fully aromatic ring but the other fused ring or rings may be aromatic or non-aromatic. The heteroaryl group may be attached at any available nitrogen or carbon atom of any ring. Illustrative examples of heteroaryl groups are pyridinyl, pyridazinyl, pyrimidyl, pyrazinyl, benzimidazolyl, triazinyl, imidazolyl, (1,2,3)-and (1,2,4)-triazolyl, pyrazinyl, tetrazolyl, furyl, pyrrolyl, thienyl, isothiazolyl, thiazolyl, isoxazolyl, and oxadiazolyl. The heteroaryl can be substituted or unsubstituted, as described herein.

The term “heterocycloalkyl” means a stable, saturated, or partially unsaturated monocyclic, bicyclic, and spiro ring system containing 3 to 7 ring members of carbon atoms and at least 1 other atom selected from nitrogen, sulfur, and oxygen. In an aspect, a heterocycloalkyl is a 5, 6, or 7-membered monocyclic ring and contains one, two, or three heteroatoms selected from nitrogen, oxygen, and sulfur. The heterocycloalkyl can be attached to the parent structure through a carbon atom or through any heteroatom of the heterocycloalkyl that results in a stable structure. Examples of such heterocycloalkyl rings are isoxazolyl, thiazolinyl, imidazolidinyl, piperazinyl, homopiperazinyl, pyrrolyl, pyrrolinyl, pyrazolyl, pyranyl, piperidyl, oxazolyl, and morpholinyl. The heterocycloalkyl can be substituted or unsubstituted, as described herein.

In any of the embodiments above, the term “hydroxy” refers to the group —OH.

In any of the embodiments above, the terms “alkoxy” and “cycloalkyloxy” embrace linear or branched alkyl and cycloalkyl groups, respectively, that are attached to a divalent oxygen. The alkyl and cycloalkyl groups are the same as described herein.

In any of the embodiments above, the term “halo” refers to a halogen selected from fluorine, chlorine, bromine, and iodine.

In any of the embodiments above, the term “carboxylato” refers to the group —C(O)OH.

In any of the embodiments above, the term “amino” refers to the group —NH2. The term “alkylamino” refers to —NHR, whereas the term “dialkylamino” refers to —NRR′. R and R′ are the same or different and each is a substituted or unsubstituted alkyl group, as described herein.

In other aspects, any substituent that is not hydrogen (e.g., C₁₋₆ alkyl, C₃₋₆ cycloalkyl, C₃₋₆ cycloalkylalkyl, aryl, arylalkyl, heteroaryl, or heterocycloalkyl) can be an optionally substituted moiety. The substituted moiety typically comprises at least one substituent (e.g., 1, 2, 3, 4, 5, 6, etc.) in any suitable position (e.g., 1-, 2-, 3-, 4-, 5-, or 6-position, etc.). When an aryl group (e.g., phenyl) is substituted with a substituent, e.g., halo, amino, alkyl, OH, alkoxy, and others, the aromatic ring hydrogen is replaced with the substituent and this can take place in any of the available hydrogens, e.g., 2, 3, 4, 5, and/or 6-position wherein the 1-position is the point of attachment of the aryl group (e.g., phenyl) in the compound of the present invention. Suitable substituents include, e.g., halo, alkyl, alkenyl, hydroxy, nitro, cyano, amino, alkylamino, alkoxy, aryloxy, aralkoxy, carboxyl, carboxyalkyl, carboxyalkyloxy, amido, alkylamido, haloalkylamido, aryl, heteroaryl, and heterocycloalkyl, each of which is described herein. In some instances, the substituent is at least one alkyl, halo, and/or haloalkyl (e.g., 1 or 2).

In any of the embodiments above, whenever a range of the number of atoms in a structure is indicated (e.g., a C₁₋₁₂, C₁₋₈, C₁₋₆ , C₁₋₄, etc.), it is specifically contemplated that any sub-range or individual number of carbon atoms falling within the indicated range also can be used. Thus, for instance, the recitation of a range of 1-8 carbon atoms (e.g., C₁₋₈), 1-6 carbon atoms (e.g., C₁₋₆ ), 1-4 carbon atoms (e.g., C₁₋₄), 1-3 carbon atoms (e.g., C₁₋₃), or 2-8 carbon atoms (e.g., C₂₋₈) as used with respect to any chemical group (e.g., alkyl, cycloalkyl, etc.) referenced herein encompasses and specifically describes 1, 2, 3, 4, 5, 6, 7, and/or 8 carbon atoms, as appropriate, as well as any sub-range thereof (e.g., 1-2 carbon atoms, 1-3 carbon atoms, 1-4 carbon atoms, 1-5 carbon atoms, 1-6 carbon atoms, 1-7 carbon atoms, 1-8 carbon atoms, 2-3 carbon atoms, 2-4 carbon atoms, 2-5 carbon atoms, 2-6 carbon atoms, 2-7 carbon atoms, 2-8 carbon atoms, 3-4 carbon atoms, 3-5 carbon atoms, 3-6 carbon atoms, 3-7 carbon atoms, 3-8 carbon atoms, 4-5 carbon atoms, 4-6 carbon atoms, 4-7 carbon atoms, 4-8 carbon atoms, etc., as appropriate).

The subscript “n” represents the number of methylene repeat units. The subscript n is either 0 or an integer from 1-5 (i.e., 1, 2, 3, 4, or 5). When n is 0, then the moiety does not contain any methylene repeat units between X² (O in (Ia)) and R⁴.

In any of the embodiments above, the phrase “salt” or “pharmaceutically acceptable salt” is intended to include nontoxic salts synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. For example, an inorganic acid (e.g., hydrochloric acid, sulfuric acid, phosphoric acid, or hydrobromic acid), an organic acid (e.g., oxalic acid, malonic acid, citric acid, fumaric acid, lactic acid, malic acid, succinic acid, tartaric acid, acetic acid, trifluoroacetic acid, gluconic acid, ascorbic acid, methylsulfonic acid, or benzylsulfonic acid), an inorganic base (e.g., sodium hydroxide, potassium hydroxide, calcium hydroxide, magnesium hydroxide, or ammonium hydroxide), an organic base(e.g., methylamine, diethylamine, triethylamine, triethanolamine, ethylenediamine, tris(hydroxymethyl)methylamine, guanidine, choline, or cinchonine), or an amino acid (e.g., lysine, arginine, or alanine) can be used. Generally, nonaqueous media such as ether, ethyl acetate, ethanol, isopropanol, or acetonitrile are typical. Lists of suitable salts are found in Remington's Pharmaceutical Sciences, 18th ed., Mack Publishing Company, Easton, PA, 1990, p. 1445, and Journal of Pharmaceutical Science, 66, 2-19 (1977). For example, they can be a salt of an alkali metal (e.g., sodium or potassium), alkaline earth metal (e.g., calcium), or ammonium of salt.

A compound of formula (I), including a compound of formula (Ia), can be prepared by any suitable synthetic method. Exemplary methods are set forth in Example 1 and FIGS. 1-15.

The methods described herein comprise administering, to a subject in need thereof, a compound of formula (I), including a compound of formula (Ia), or a pharmaceutically acceptable salt thereof in the form of a pharmaceutical composition. In particular, a pharmaceutical composition will comprise at least one compound of formula (I) or a pharmaceutically acceptable salt thereof and a pharmaceutically acceptable carrier. The pharmaceutically acceptable excipients described herein, for example, vehicles, adjuvants, carriers or diluents, are well-known to those who are skilled in the art and are readily available to the public. Typically, the pharmaceutically acceptable carrier is one that is chemically inert to the active compounds and one that has no detrimental side effects or toxicity under the conditions of use.

The pharmaceutical compositions can be administered by via any suitable formulation, including oral, sublingual, transdermal, subcutaneous, topical, absorption through epithelial or mucocutaneous linings, intravenous, intranasal, intraarterial, intraperitoneal, intramuscular, intratumoral, peritumoral, intraperitoneal, intrathecal, rectal, vaginal, and aerosol formulations. In some aspects, the pharmaceutical composition is administered orally or intravenously.

In accordance with any of the embodiments, the compound of formula (I) or a pharmaceutically acceptable salt thereof can be administered orally to a subject in need thereof. Formulations suitable for oral administration can consist of (a) liquid solutions, such as an effective amount of the compound dissolved in diluents, such as water, saline, or orange juice and include an additive, such as cyclodextrin (e.g., α-, β-, or γ-cyclodextrin, hydroxypropyl cyclodextrin) or polyethylene glycol (e.g., PEG400); (b) capsules, sachets, tablets, lozenges, and troches, each containing a predetermined amount of the active ingredient, as solids or granules; (c) powders; (d) suspensions in an appropriate liquid; and (e) suitable emulsions and gels. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound of formula (I) or a salt thereof can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

The parenteral formulations will typically contain from about 0.5 to about 25% by weight of the compound(s) of formula (I) in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Topically applied compositions are generally in the form of liquids (e.g., mouthwash), patches creams, pastes, lotions and gels. Topical administration includes application to the skin and oral mucosa, which includes the oral cavity, oral epithelium, palate, gingival, and the nasal mucosa. In some embodiments, the composition contains at least one active component and a suitable vehicle or carrier. It may also contain other components, such as an anti-irritant. The carrier can be a liquid, solid or semi-solid. In embodiments, the composition can be a dispersion, emulsion, gel, lotion or cream vehicle for the various components. In an embodiment, the primary vehicle is water or a biocompatible solvent that is substantially neutral or that has been rendered substantially neutral. The liquid vehicle can include other materials, such as buffers, alcohols, glycerin, and mineral oils with various emulsifiers or dispersing agents as known in the art to obtain the desired pH, consistency and viscosity. It is possible that the compositions can be produced as solids, such as powders or granules. The solids can be applied directly or dissolved in water or a biocompatible solvent prior to use to form a solution that is substantially neutral or that has been rendered substantially neutral and that can then be applied to the target site. In embodiments of the invention, the vehicle for topical application to the skin can include water, buffered solutions, various alcohols, glycols such as glycerin, lipid materials such as fatty acids, mineral oils, phosphoglycerides, collagen, gelatin and silicone based materials.

The dose administered to the mammal, particularly a human and other mammals, in accordance with the present invention should be sufficient to affect the desired response. One skilled in the art will recognize that dosage will depend upon a variety of factors, including the age, condition or disease state, predisposition to disease, genetic defect or defects, and body weight of the mammal. The size of the dose will also be determined by the route, timing and frequency of administration as well as the existence, nature, and extent of any adverse side-effects that might accompany the administration of a particular inhibitor and the desired effect. It will be appreciated by one of skill in the art that various conditions or disease states may require prolonged treatment involving multiple administrations.

The inventive methods comprise administering an effective amount of a compound of formula (I) or a pharmaceutically acceptable salt thereof. The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of an estrogen-mediated diseased (e.g., cancer) in a mammal. Furthermore, the treatment or prevention provided by the inventive method can include treatment or prevention of one or more conditions or symptoms of the disease being treated or prevented. For example, treatment or prevention can include promoting the regression of at least one symptom of the disease, such as a tumor. Also, for purposes herein, “prevention” can encompass delaying the onset of the disease, or a symptom or condition thereof. Alternatively or additionally, “prevention” can encompass preventing or delaying the recurrence of the disease, or a symptom or condition thereof.

An “effective amount” means an amount sufficient to show a meaningful benefit in an individual, e.g., promoting at least one aspect of tumor cell cytotoxicity (e.g., inhibition of growth, inhibiting survival of a cancer cell, reducing proliferation, reducing size and/or mass of a tumor (e.g., solid tumor)), or treatment, healing, prevention, delay of onset, halting, or amelioration of other relevant medical condition(s) associated with a particular estrogen-mediated disease. The meaningful benefit observed in the subject can be to any suitable degree (10, 20, 30, 40, 50, 60, 70, 80, 90% or more). Effective amounts may vary depending upon the biological effect desired in the individual, condition to be treated, and/or the specific characteristics of the compound of formula (I) or a pharmaceutically acceptable salt thereof, and the individual. In this respect, any suitable dose of the compound of formula (I) or a pharmaceutically acceptable salt thereof can be administered to the subject (e.g., human), according to the type of disease to be treated (e.g., breast cancer). Various general considerations taken into account in determining the “effective amount” are known to those of skill in the art and are described, e.g., in Gilman et al., eds., Goodman and Gilman's: The Pharmacological Bases of Therapeutics, 8th ed., Pergamon Press, 1990; and Remington's Pharmaceutical Sciences, 17th Ed., Mack Publishing Co., Easton, Pa., 1990, each of which is herein incorporated by reference. The dose of the compound of formula (I) or a pharmaceutically acceptable salt thereof desirably comprises about 0.01 mg per kilogram (kg) of the body weight of the subject (mg/kg) or more (e.g., about 0.05 mg/kg or more, 0.1 mg/kg or more, 0.5 mg/kg or more, 1 mg/kg or more, 2 mg/kg or more, 5 mg/kg or more, 10 mg/kg or more, 15 mg/kg or more, 20 mg/kg or more, 30 mg/kg or more, 40 mg/kg or more, 50 mg/kg or more, 75 mg/kg or more, 100 mg/kg or more, 125 mg/kg or more, 150 mg/kg or more, 175 mg/kg or more, 200 mg/kg or more, 225 mg/kg or more, 250 mg/kg or more, 275 mg/kg or more, 300 mg/kg or more, 325 mg/kg or more, 350 mg/kg or more, 375 mg/kg or more, 400 mg/kg or more, 425 mg/kg or more, 450 mg/kg or more, or 475 mg/kg or more) per day. Typically, the dose will be about 500 mg/kg or less (e.g., about 475 mg/kg or less, about 450 mg/kg or less, about 425 mg/kg or less, about 400 mg/kg or less, about 375 mg/kg or less, about 350 mg/kg or less, about 325 mg/kg or less, about 300 mg/kg or less, about 275 mg/kg or less, about 250 mg/kg or less, about 225 mg/kg or less, about 200 mg/kg or less, about 175 mg/kg or less, about 150 mg/kg or less, about 125 mg/kg or less, about 100 mg/kg or less, about 75 mg/kg or less, about 50 mg/kg or less, about 40 mg/kg or less, about 30 mg/kg or less, about 20 mg/kg or less, about 15 mg/kg or less, about 10 mg/kg or less, about 5 mg/kg or less, about 2 mg/kg or less, about 1 mg/kg or less, about 0.5 mg/kg or less, or about 0.1 mg/kg or less). Any two of the foregoing endpoints can be used to define a close-ended range, or a single endpoint can be used to define an open-ended range.

For purposes of the present invention, the term “subject” preferably is directed to a mammal. Mammals include, but are not limited to, the order Rodentia, such as mice, and the order Lagomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perissodactyla, including Equines (horses). It is most preferred that the mammals are of the order Primates, Cebids, or Simioids (monkeys) or of the order Anthropoids (humans and apes). An especially preferred mammal is a human.

In an aspect, a compound of formula (I) inhibits estrogen receptor alpha (ERα). Accordingly, the invention provides a method of inhibiting estrogen receptor alpha (ERα) in a cell (e.g., a breast cancer cell) comprising contacting the cell with a compound of formula (I), including a compound of formula (Ia), a stereoisomer thereof, and/or a pharmaceutically acceptable salt thereof. The ERα activity can be measured by any method, including assays described herein.

Because the compound of formula (I) inhibits or suppresses estrogen production, the compound is considered useful in treating an estrogen-mediated disease requiring inhibition of estrogen receptor alpha (ERa). See, for example, Deroo et al. (J. Clin Invest, 2006, 116(3), 561-570). The method comprises administering to a subject in need thereof the compound of formula (I) or a pharmaceutically acceptable salt thereof. The estrogen-mediated disease is any disease that is treatable by inhibition of ERα, such as an ER-positive cancer, osteoporosis, vulvovaginal atrophy, hormone replacement therapy (HRT), one or more symptoms of menopause (e.g., hot flashes, bone loss, vaginal dryness, night sweats, mood swings), obesity, and a fibroid.

ERα is overexpressed in many cancers. As such, inhibition of ERa is considered to be a viable treatment of cancers that overexpress ERα, particularly breast cancer, ovarian cancer, colon cancer, prostate cancer, lung cancer, and endometrial cancer. Thus, the invention is further directed to a method of treating an ER-positive (including mutated ER) cancer comprising administering to a subject in need thereof a compound of formula (I), including a compound of formula (Ia), a stereoisomer thereof, and/or a pharmaceutically acceptable salt thereof. The ER-positive cancer is any suitable cancer, including SERM-resistant ER+ luminal cancer, hormone refractory ER+ cancer, ER+ hormone-resistant cancer, hormone insensitive cancer, and cancer with a somatic mutation to ESR1 (the gene for ERα)—any of which can be associated with any suitable tissue, such as tissue of the breast, ovaries, colon, rectum, prostate, lung, or endometrial lining of the uterus. In a preferred embodiment of the method, the cancer to be treated is breast cancer. Anti-cancer activity can be measured by any suitable method, including the assays described herein.

In certain embodiments of this method, the compound of formula (I) or a pharmaceutically acceptable salt thereof can be co-administered with one or more therapeutic agents (e.g., a chemotherapeutic agent) and/or radiation therapy. In an aspect, the method comprises administering an amount of a compound of formula (I) or a pharmaceutically acceptable salt thereof that is effective to sensitize cells (e.g., cancer cells) to one or more therapeutic regimens (e.g., chemotherapy or radiation therapy). The terms “co-administered” or “co-administration” refer to simultaneous or sequential administration. A compound of formula (I) or a pharmaceutically acceptable salt thereof can be administered before, concurrently with, or after administration of another therapeutic agent (e.g., a chemotherapeutic agent).

One or more (e.g., one, two, three, four, or more) therapeutic agents can be administered. In this regard, the present invention is directed a pharmaceutical composition comprising a pharmaceutically acceptable carrier and a combination of the compound of formula (I) or a pharmaceutically acceptable salt thereof and at least one therapeutic agent (e.g., chemotherapeutic agent). The therapeutic agent is any agent suitable for treating an estrogen-mediated disease, particularly, diseases in which a subject has been become or is becoming resistant to conventional therapies. In some aspects of the invention, the therapeutic agent can be a hormonal agent or an anti-cancer agent (e.g., chemotherapeutic agent).

Examples of a hormonal agent include estrogen (e.g., estradiol, estriol, and estrone), progestin, and progesterone.

Examples of anti-cancer agents include platinum compounds (e.g., cisplatin, carboplatin, oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, bendamustine), antitumor antibiotics (e.g., daunorubicin, doxorubicin, idarubicin, epirubicin, mitoxantrone, bleomycin, mitomycin C, plicamycin, dactinomycin), taxanes (e.g., paclitaxel and docetaxel), antimetabolites (e.g., 5-fluorouracil, cytarabine, pemetrexed, thioguanine, floxuridine, capecitabine, and methotrexate), nucleoside analogues (e.g., fludarabine, clofarabine, cladribine, pentostatin, nelarabine), topoisomerase inhibitors (e.g., topotecan and irinotecan), hypomethylating agents (e.g., azacitidine and decitabine), proteosome inhibitors (e.g., bortezomib), epipodophyllotoxins (e.g., etoposide and teniposide), DNA synthesis inhibitors (e.g., hydroxyurea), vinca alkaloids (e.g., vincristine, vindesine, vinorelbine, and vinblastine), tyrosine kinase inhibitors (e.g., imatinib, dasatinib, nilotinib, sorafenib, sunitinib), monoclonal antibodies (e.g., rituximab, cetuximab, panitumumab, tositumomab, trastuzumab, alemtuzumab, gemtuzumab ozogamicin, bevacizumab), nitrosoureas (e.g., carmustine, fotemustine, and lomustine), enzymes (e.g., L-Asparaginase), biological agents (e.g., interferons and interleukins), hexamethylmelamine, mitotane, angiogenesis inhibitors (e.g., thalidomide, lenalidomide), steroids (e.g., prednisone, dexamethasone, and prednisolone), a CDK4/6 inhibitor (e.g., abemaciclib, palbociclib, ribociclib), anti-cancer hormonal agents (e.g., tamoxifen, fulvestrant, raloxifene, leuprolide, bicalutamide, granisetron, flutamide, goserelin), aromatase inhibitors (e.g., exemestane, letrozole, and anastrozole), arsenic trioxide, tretinoin, nonselective cyclooxygenase inhibitors (e.g., nonsteroidal anti-inflammatory agents, salicylates, aspirin, piroxicam, ibuprofen, indomethacin, naprosyn, diclofenac, tolmetin, ketoprofen, nabumetone, oxaprozin), selective cyclooxygenase-2 (COX-2) inhibitors, cellular immunotherapy (e.g., chimeric antigen receptor T cell therapy, tumor-infiltrating lymphocyte therapy), or any combination thereof

In some embodiments, the anti-cancer agent is at least one of a CDK4/6 inhibitor (e.g., abemaciclib, palbociclib, ribociclib), an anti-cancer hormonal agent (e.g., tamoxifen, fulvestrant, raloxifene, leuprolide, bicalutamide, granisetron, flutamide, goserelin), and an aromatase inhibitor (e.g., exemestane, letrozole, anastrozole).

The invention is further illustrated by the following aspects.
Aspect (1) A compound of formula (I) or a pharmaceutically acceptable salt thereof:

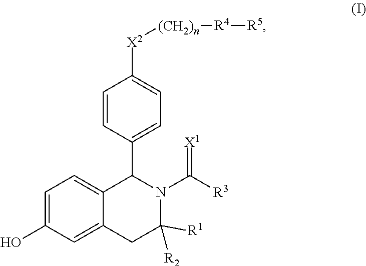

wherein
X¹ is O or S;
X² is a bond, alkenyl, O, S, or NR₆;
R¹ is selected from H and C₁₋₆ alkyl;

R² is C₁₋₆ alkyl; or

R¹ and R² together form C₃₋₆ cycloalkyl,

R³ is C₂₋₁₂ alkyl or phenyl optionally substituted with at least one substituent selected from C₁₋₆ alkyl, C₃₋₆ cycloalkyl, hydroxy, alkoxy, cycloalkoxy, halo, and amino; or

R³ is phenyl with two substituents that together with the phenyl form an optionally substituted bicyclic nitrogen-containing heteroaryl;

R⁴ is a nitrogen-containing C₃₋₇ heterocycloalkyl, —NR⁷—;

R⁵, R⁶, and R⁷ are the same or different and each is a hydrogen or C₁₋₆ alkyl; and

n is 0 or an integer of 1 to 5,

wherein the C₁₋₆ alkyl and C₃₋₆ cycloalkyl can be substituted with one or more substituents selected from hydroxy, halo, alkoxy, alkyl, cycloalkyl, heterocycloalkyl, aryl, heteroaryl, amino, alkylamino, dialkylamino, and carboxylato.
Aspect (2) The compound of aspect (1) or a pharmaceutically acceptable salt thereof, wherein X¹ and X² are both O.
Aspect (3) The compound of aspect (1) or (2) or a pharmaceutically acceptable salt thereof, wherein R¹ and R² together form cyclopropyl.
Aspect (4) The compound of any one of aspects (1)-(3) or a pharmaceutically acceptable salt thereof, wherein R³ is neopentyl.
Aspect (5) The compound of any one of aspects (1)-(3) or a pharmaceutically acceptable salt thereof, wherein R³ is phenyl optionally substituted with at least one sub stituent selected from C₁₋₆ alkyl, haloalkyl, and halo.
Aspect (6) The compound of any one of aspects (1)-(3) or a pharmaceutically acceptable salt thereof, wherein R³ is substituted phenyl, wherein two substituents together with the phenyl group form indazolyl, indolizinyl, pyrazolo[1,5-a]pyridinyl, or imidazo[1,5-a]pyridinyl, each of which is optionally substituted.
Aspect (7) The compound of any one of aspects (1)-(6) or a pharmaceutically acceptable salt thereof, wherein R⁴ is 3-azetidinyl, 1-pyrrolidinyl, 3-pyrrolidinyl, 1-piperidinyl, 4-piperidinyl, 1-piperazinyl, or 1-azepanyl.
Aspect (8) The compound of any one aspects (1)-(6) or a pharmaceutically acceptable salt thereof, wherein R⁴ is —NH— or —N(C₁₋₃ alkyl)-.
Aspect (9) The compound of any one of aspects (1)-(8) or a pharmaceutically acceptable salt thereof, wherein R⁵ is C₁₋₃ alkyl.
Aspect (10) The compound of any one of aspects (1)-(9) or a pharmaceutically acceptable salt thereof, wherein n is 0 or 2.
Aspect (11) The compound of aspect (1), wherein the compound of formula (I) is selected from

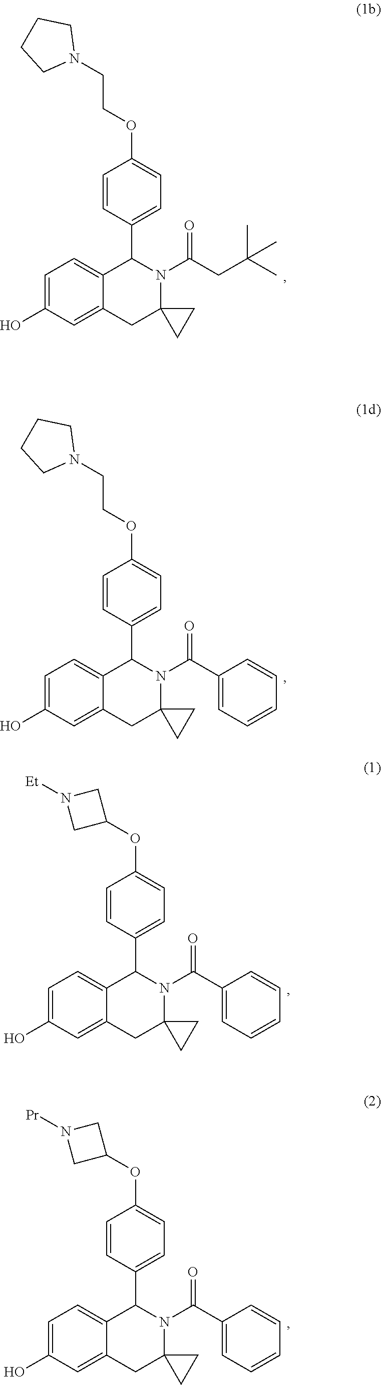

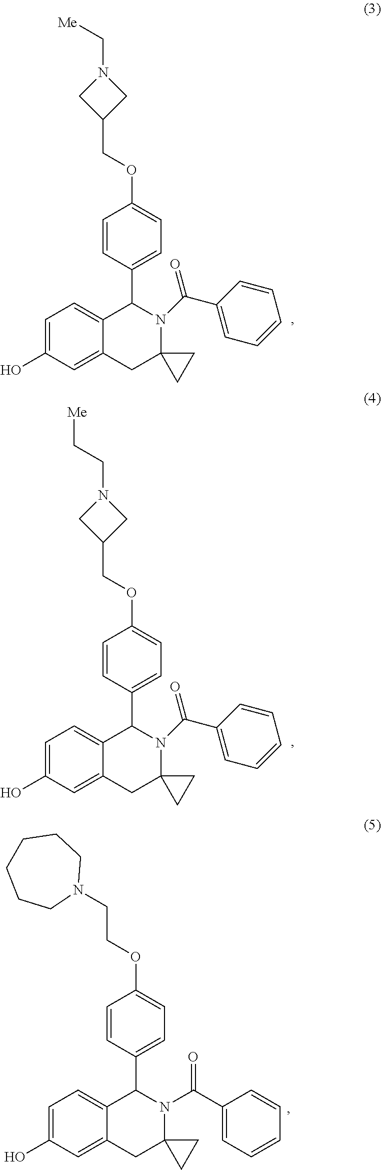

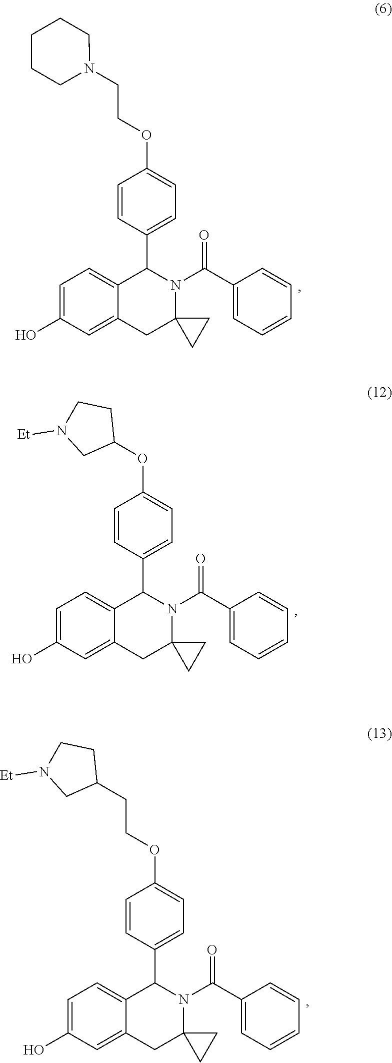 [SMILES: O=C(c1ccccc1)N1C(c2ccc(OCCN3CCCCC3)cc2)c2ccc(O)cc2CC12CC2 ; CCN1CCC(Oc2ccc(C3c4ccc(O)cc4CC4(CC4)N3C(=O)c3ccccc3)cc2)C1 ; CCN1CCC(CCOc2ccc(C3c4ccc(O)cc4CC4(CC4)N3C(=O)c3ccccc3)cc2)C1]

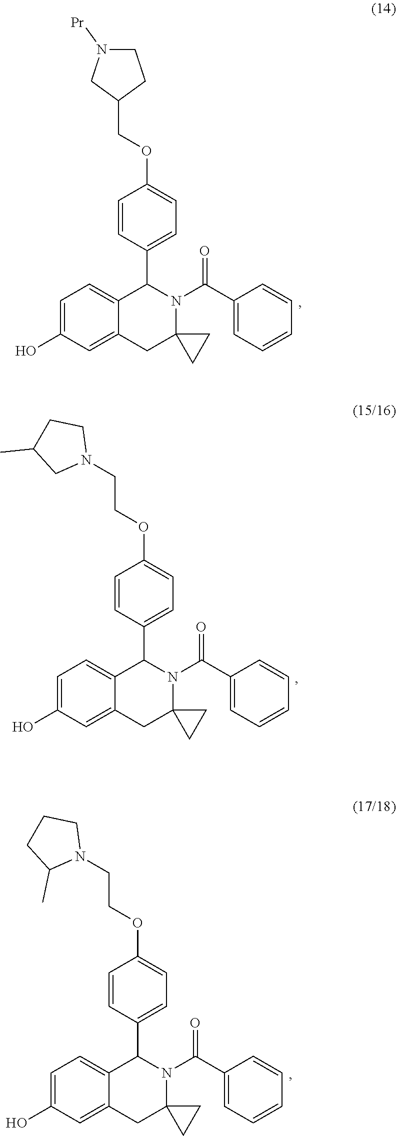

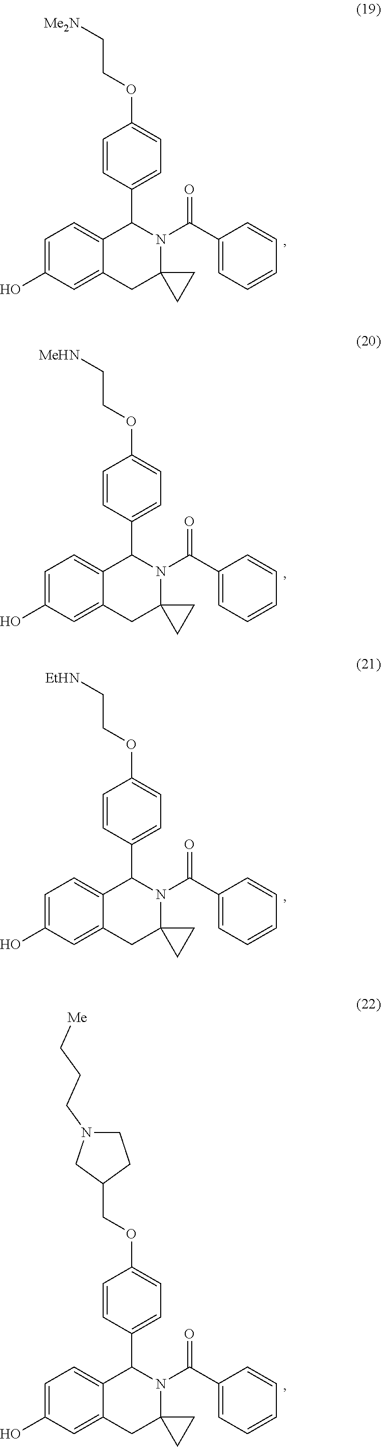

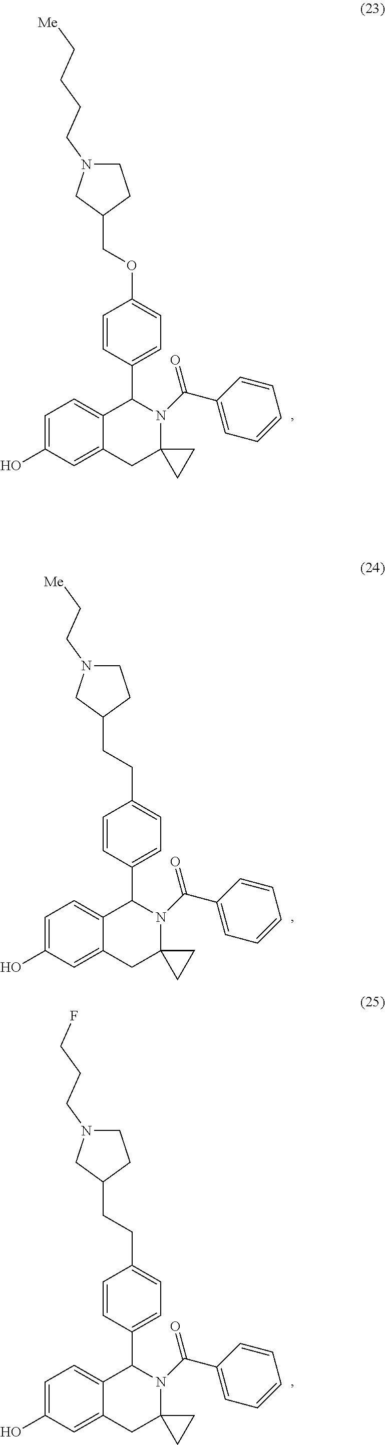 [SMILES: CCCCCN1CCC(COc2ccc(C3c4ccc(O)cc4CC4(CC4)N3C(=O)c3ccccc3)cc2)C1 ; CCCN1CCC(CCc2ccc(C3c4ccc(O)cc4CC4(CC4)N3C(=O)c3ccccc3)cc2)C1 ; O=C(c1ccccc1)N1C(c2ccc(CCC3CCN(CCCF)C3)cc2)c2ccc(O)cc2CC12CC2]

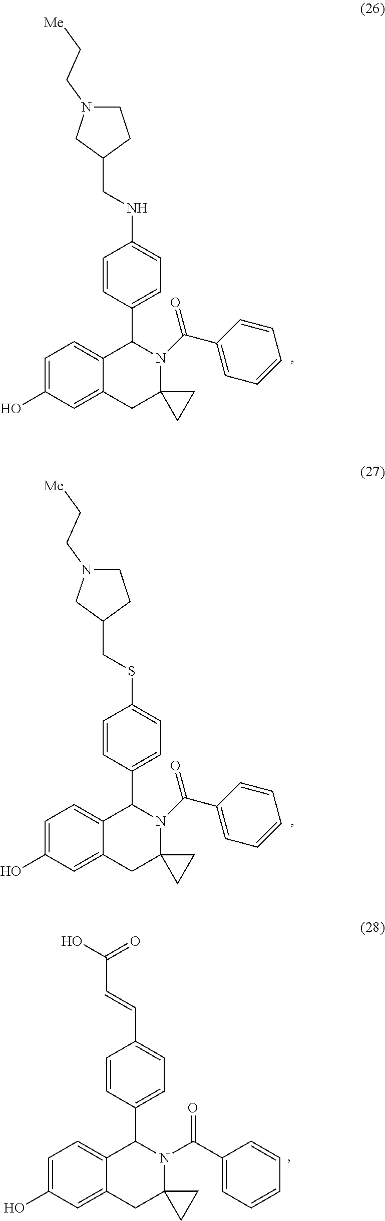 [SMILES: CCCN1CCC(CNc2ccc(C3c4ccc(O)cc4CC4(CC4)N3C(=O)c3ccccc3)cc2)C1 ; CCCN1CCC(CSc2ccc(C3c4ccc(O)cc4CC4(CC4)N3C(=O)c3ccccc3)cc2)C1 ; O=C(O)/C=C/c1ccc(C2c3ccc(O)cc3CC3(CC3)N2C(=O)c2ccccc2)cc1]

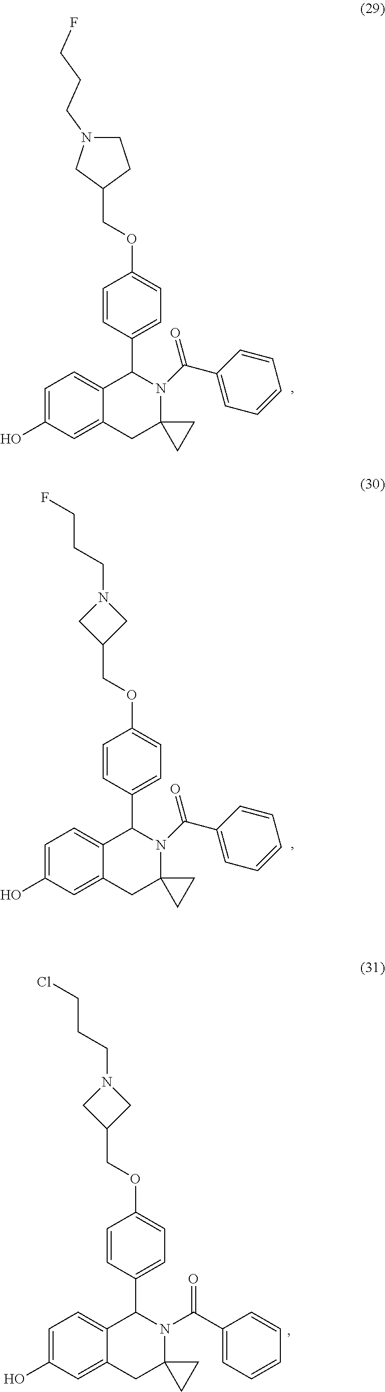 [SMILES: O=C(c1ccccc1)N1C(c2ccc(OCC3CCN(CCCF)C3)cc2)c2ccc(O)cc2CC12CC2 ; O=C(c1ccccc1)N1C(c2ccc(OCC3CN(CCCF)C3)cc2)c2ccc(O)cc2CC12CC2 ; O=C(c1ccccc1)N1C(c2ccc(OCC3CN(CCCCl)C3)cc2)c2ccc(O)cc2CC12CC2]

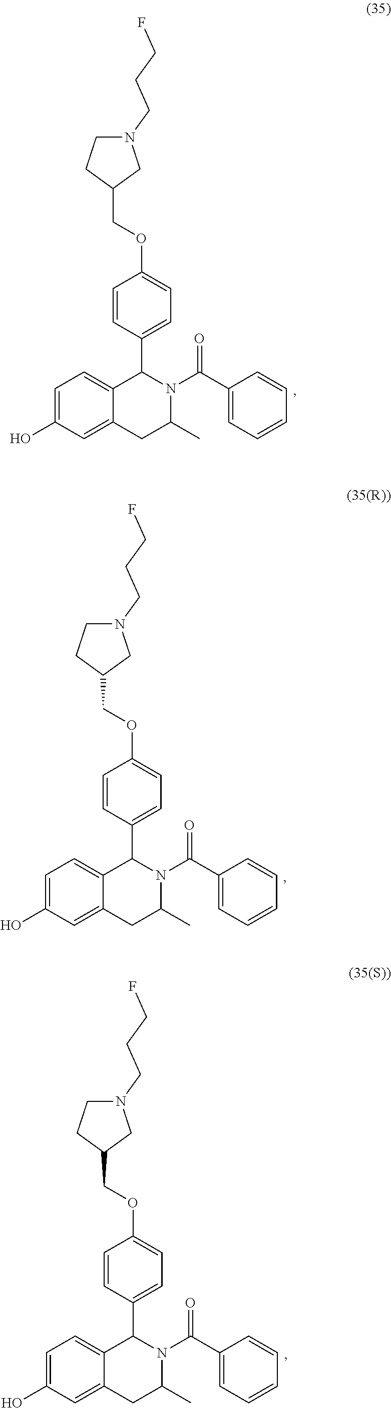

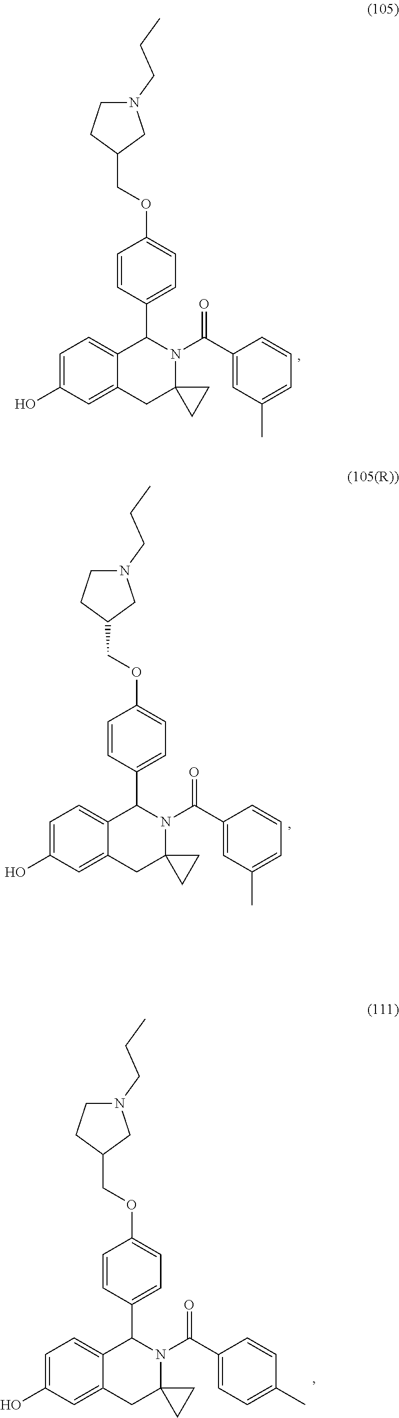

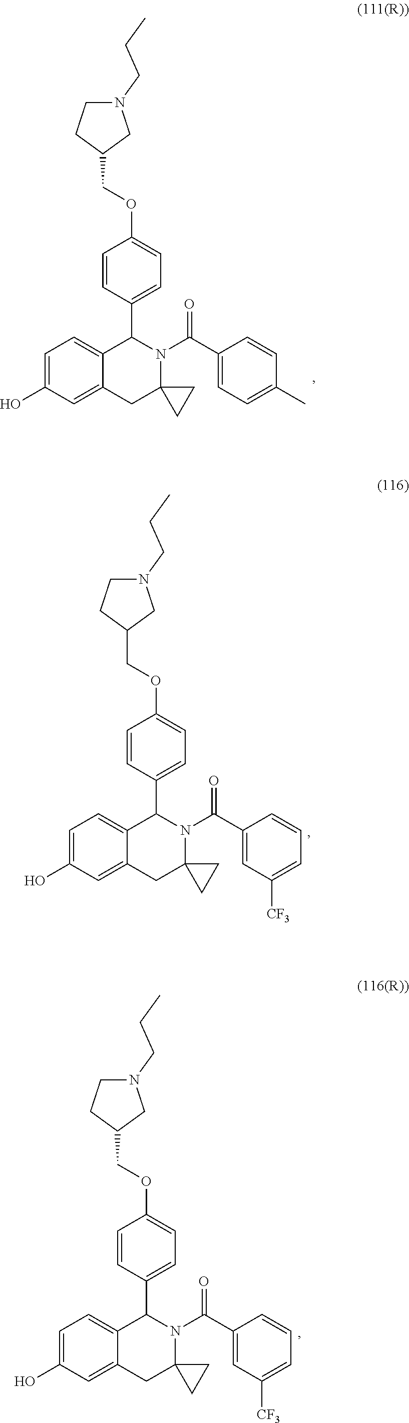

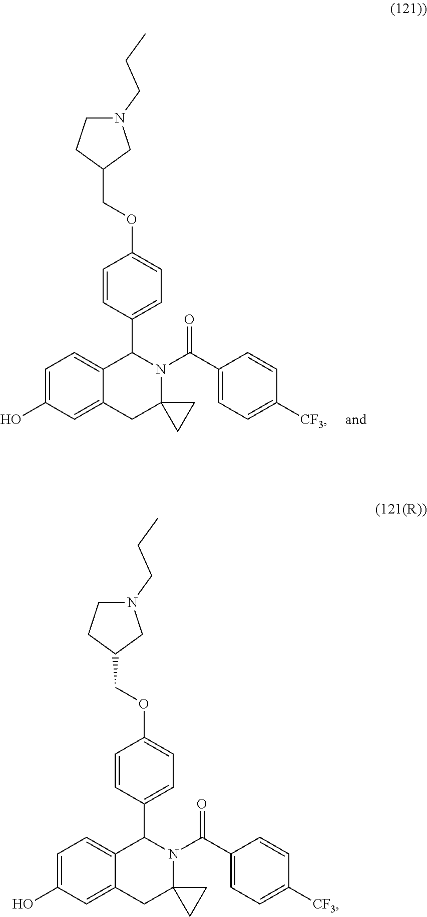

or a stereoisomer thereof and/or a pharmaceutically acceptable salt thereof.
Aspect (12) A pharmaceutical composition comprising a compound of any one of aspects (1)-(11) or a pharmaceutically acceptable salt thereof and a pharmaceutically acceptable carrier.
Aspect (13) A method of treating breast cancer comprising administering to a subject in need thereof the compound of any one of aspects (1)-(11) or a pharmaceutically acceptable salt thereof
Aspect (14) A method of inhibiting estrogen receptor alpha (ERα) in a cell comprising contacting the cell with the compound of any one of aspects (1)-(11) or a pharmaceutically acceptable salt thereof.

The following examples further illustrate the invention but, of course, should not be construed as in any way limiting its scope.

### EXAMPLES

Reagents and solvents were used as received from commercial suppliers. ¹H NMR spectra were obtained on a Bruker AVANCE™ 400 spectrometer (Billerica, MA) at 400 MHz with tetramethylsilane as an internal standard for proton spectra. Thin-layer chromatography (TLC) was performed using Merck TLC silica gel 60 F₂₅₄ plates (Kenilworth, NJ). Visualization of TLC plates was performed using UV light (220, 230 nm). The mass spectra were obtained on a Waters ACQUITY™ LC-MS spectrometer (Milford, MA) using Electrospray Ionization. High performance liquid chromatography (HPLC) analysis were performed with Method A.

The HPLC method included the following: a Zorbax Eclipse Plus C18 column (100×4.6 mm, 3.5 μm) (Agilent, Santa Clara, CA), a purge flow of 0.8 mL/ min. Mobile phase A comprised 0.05% trifluoroacetic acid (TFA) in water, and mobile phase B comprised 0.05% TFA in acetonitrile. The method gradient for Method A is set forth in Table 1.

TABLE 1
Method Gradient

| Time | Flow | | |
|---|---|---|---|
| (min) | (mL/min) | % A | % B |
| 0 | 1.0 | 95.0 | 5.0 |
| 2 | 1.0 | 95.0 | 5.0 |
| 8 | 1.0 | 30.0 | 70.0 |
| 10 | 1.0 | 5.0 | 95.0 |
| 12 | 1.0 | 5.0 | 95.0 |
| 12.1 | 1.0 | 95.0 | 5.0 |
| 15 | 1.0 | 95.0 | 5.0 |

HPLC purification analysis was performed using a WATERS™ mass-based auto purification system (Milford, MA) with a binary solvent system A and B using a gradient elution. The prep HPLC purification method (Method A) included the following: a GEMINI™ NX C18 column (150×30 mm, 10 μm) (Phenomenex, Torrance, CA), with a flow rate of 30 mL/min, an injection volume of 500 μL, a run time of 20 min, and detection at 220 nm and 254 nm. Mobile phase A comprised 10 mM ammonium formate in water, and mobile phase B comprised acetonitrile. The method gradient used is set forth in Table 2.

TABLE 2
Method Gradient

| Time | Flow | | |
|---|---|---|---|
| (min) | (mL/min) | % A | % B |
| 0 | 30.0 | 90.0 | 10 |
| 1 | 30.0 | 90.0 | 10 |
| 10 | 30.0 | 50.0 | 50 |
| 15 | 30.0 | 20.0 | 80 |
| 15.5 | 30.0 | 5.0 | 95.0 |
| 17.5 | 30.0 | 5.0 | 95.0 |
| 18.0 | 30.0 | 90.0 | 10.0 |
| 20.0 | 30.0 | 90.0 | 10.0 |

The preparative supercritical fluid chromatography (SFC) method conditions used are set forth in Table 3.

TABLE 3

| | |
|---|---|
| Column | CHIRALCEL ™ OX-H (250 mm × 21 |
| | mm) 5 μm (Daicel Corp., Japan) |
| Total Flow | 85 g-40% |
| Mobile Phase A | 51 g/min (CO₂) |
| Mobile Phase B | 34 mL/min (0.3% NH₃ in methanol) |
| Automated Back Pressure Regulator (ABPR) | 100 bar (10,000 kPa) |
| Detection | UV @ 220 nm |
| Run time | 20 min |
| Stocking time | 12 min |
| Loading | 10 mg/injection |
| Dilution | methanol (100%) |

The analytical SFC method conditions (Method B) used are set forth in Table 4.

TABLE 4

| | |
|---|---|
| Column | CHIRALCEL ™ OX-H(150 mm × 4.6 mm) 5 μm |
| Total Flow | 4 g-40% |
| Mobile Phase A | 2.4 g/min (CO₂) |
| Mobile Phase B | 1.6 mL/min (0.3% diethanolamine (DEA) in methanol) |
| ABPR | 1500 psi (10,342 kPa) |
| Column Temp | 40° C. |
| Detection | UV @ 220 nm |
| Run time | 15 min |
| Dilution | methanol (100%) |

### Example 1

This example demonstrates a synthetic approach to preparing compounds of formula (I).

It was determined that compound H-8 is a suitable starting material for the compounds of formula (I). FIG. 1 is a chemical scheme of preparing compound H-8 starting from commercially available 2-(3-methoxyphenyl)acetonitrile (compound H-1). H-1 was reacted with titanium isopropaxide, BF₃·EtO and EtMgBr to afford H-2. H-2 was reacted with H-3, TFA, toluene in Microwave to achieve H-4. H-4 was reacted with H-5, TEA to afford H-6. H-6 wastreated with 1.0 M BBr₃ to afford demethylated compound H-7 which was protected with benzyl treating with benzyl bromide to afford H-8.

For the preparation of H-2: H-1 (10.00 g, 68.02 mmol) in dry tetrahydrofuran (THF) (300 mL) was charged with titanium tetra isopropoxide (24.00 mL, 81.62 mmol) followed by the addition of EtMgBr (136.0 mL, 136.05 mmol, 1.0 M in THF) at room temperature under argon atmosphere. The reaction was exothermic during the Grignard addition. The resulting mixture was stirred for 1 h at room temperature. BF₃·Et2O (20.00 mL, 136.05 mmol) was added to the reaction mixture and stirred for 1 h. The reaction mixture was poured into a cold aqueous solution of (10%) NaOH (100.0 mL) and diluted with EtOAc (1000 mL). The resulting reaction mixture was filtered, and the organic layer was washed with water (2×100 mL) and brine (2×100 mL). The resulting reaction mixture was dried over Na₂SO₄, filtered, and concentrated under reduced pressure to obtain H-6 [11.50 g (crude), AMRI lot # IN-GUM-C-178] as light yellow oil; ESI-MS: m/z=(M+H)⁺178.24.

For the preparation of H-4: H-3 [3.00 g, 12.93 mmol, AMRI lot # IN-GUM-C-146] in toluene (20.0 mL) was charged with H-2 [4.57 g, 25.86 mmol, AMRI lot # IN-GUM-C-178] TFA (15.0 mL) at room temperature. The resulting reaction mixture was stirred at 140° C. for 45 min in microwave. The reaction mixture was cooled to room temperature, diluted with EtOAc(500 mL), and washed with sat. aq. NaHCO3 (3×150 mL) and brine (3×150 mL), dried over Na₂SO₄, filtered, and concentrated under reduced pressure to obtain crude material. The obtained crude material was purified by flash chromatography using silica gel (100-200 mesh) and eluted with (0-5%) MeOH in CH₂Cl₂. Combined column fractions were concentrated under reduced pressure to afford H-4 [3.80 g, 75% (yield is combined four batchs), AMRI lot # IN-GUM-D-10) as brown oil.

¹H NMR (400 MHz, CDCl₃): δ 7.65 (d, J=8.4 Hz, 2H), 7.09 (d, J=8.4 Hz, 2H), 6.64-6.59 (m, 2H), 6.49 (d, J=8.8 Hz, 1H) 4.87 (s, 1H), 3.68 (s, 3H), 3.25 (bd, J=16.4 Hz,1H), 2.66 (b s, 1H), 2.28 (bd, J=16.4 Hz,1H), 0.57-0.48 (m, 3H), 0.33-0.30 (m, 1H); ESI-MS: m/z=(M+H)⁺392.02.

For the preparation of H-6: H-4 [3.80 g, 9.71 mmol, AMRI lot # IN-GUM-D-10] in CH₂C₂ (50 mL) was charged with triethylamine (TEA) (2.70 mL, 19.43 mmol) followed by H-5 (1.30 mL, 11.66 mmol) at 0° C., under argon atmosphere. The reaction mixture was stirred for 4 h and diluted with CH₂Cl₂ (150 mL). The organic layer was washed with water (2×50 mL) and brine (2×50 mL). The resulting mixture was dried over Na₂SO₄, filtered, and concentrated under reduced pressure to obtain crude material. The obtained crude material was purified by flash chromatography using silica gel (100-200 mesh) and eluted with (25-30%) EtOAc in hexanes. Combined column fractions were concentrated under reduced pressure to afford H-6 [3.80 g, 79.00%, AMRI lot # IN-GUM-D-12] as an off white foam solid.

¹H NMR (400 MHz, CDCl₃): δ 7.58 (d, J=8.4 Hz, 2H), 7.48-7.38 (m, 5H), 7.16 (d, J=8.0 Hz, 2H), 7.01 (d, J=8.4 Hz, 2H), 6.82 (dd, J=8.4 Hz, 2.0 Hz, 1H), 6.71(s, 1H), 3.84 (s, 3H), 3.73 (bd, J=16.8 Hz,1H), 2.17 (bd, J=16.8 Hz,1H), 0.30-0.28 (m, 3H), −0.033-0.028 (m, 1H); ESI-MS: m/z=(M+H)⁺496.03.

For the preparation of H-7: H-δ [3.80 g, 7.67 mmol, AMRI lot # IN-GUM-D-12] in CH₂Cl₂ (50 mL) was charged with BBr₃ (20.0 mL, 19.19 mmol, 1.0 M in CH₂Cl₂), at 0° C., under argon atmosphere. The reaction mixture was stirred for 2 h and quenched with MeOH (10 mL) at 0° C. The resultant reaction mixture was stirred at room temperature for 1 h. After 1 h, the reaction mixture was directly concentrated under reduced pressure to obtain crude material. The obtained crude was purified by flash chromatography using silica gel (100-200 mesh) and eluted with 0-2% MeOH in CH₂Cl₂. Combined column fractions were concentrated under reduced pressure to afford H-7 [3.72 g, 98.00%, AMRI lot # IN-GUM-D-15] as an off white foam solid

¹H NMR (400 MHz, CDCl₃): δ 9.45 (s, 1H), 6 7.67 (d, J=8.0 Hz, 2H), 7.45 (bs, 5H), 7.08 (d, J=8.0 Hz, 2H), 6.91 (d, J=8.4 Hz, 1H), 6.79 (bs, 1H), 6.67 (dd, J=8.4 Hz, 2.4 Hz, 1H), 6.64 (s, 1H), 3.62 (bd, J=16.8 Hz,1H), 2.20 (bd, J=16.8 Hz,1H), 0.29 (bs, 2H), 0.13 (bs, 1H), -0.028-0.033 (m, 1H); ESI-MS: m/z=(M+H)⁺482.01.

For the preparation of H-8: H-7 [3.70 g, 7.90 mmol, AMRI lot # IN-GUM-D-15] in dimethylformamide (DMF) (50 mL) was charged with K₂CO₃ (1.60 g, 11.85 mmol), followed by benzyl bromide (1.18 mL, 9.87 mmol) at room temperature under argon atmosphere. The reaction mixture was stirred for 24 h and then was poured in ice cold water (200 mL). The product was extracted with EtOAc (3×50 mL). The combined organic layer was washed with cold brine (2×50 mL), water (2×50 mL), dried over Na₂SO₄, filtered, and concentrated under reduced pressure to obtain crude material. The obtained crude material was purified by flash chromatography using silica gel (100-200 mesh) and eluted with (15-20%) EtOAc in hexanes. Combined column fractions were concentrated under reduced pressure to afford H-8 [3.20 g, 78.0%, AMRI lot # IN-GUM-D-17] as an off white solid.

¹NMR (400 MHz, CDCl₃): δ 7.58 (d, J=8.4 Hz, 2H), 7.47-7.35 (m, 11H), 7.17 (d, J=8.0 Hz, 2H), 7.01 (d, J=8.4 Hz, 1H), 6.89 (dd, J=8.4 Hz, 2.4 Hz, 1H), 6.80 (d, J=2.4 Hz, 1H), 5.09 (s, 2H), 3.72 (bd, J=16.8 Hz,1H), 2.16 (bd, J=16.8 Hz,1H), 0.39-0.20 (m, 3H), -0.03-−0.058 (m, 1H); ESI-MS: m/z =(M+H)⁺572.05.

### Example 2

This example demonstrates a synthesis of compound 1 in an embodiment of the invention.

FIG. 2 is a chemical scheme of preparing compound 1 from H-8. H-8 was reacted with H-9 in sealed tube to produce H-10. H-10 was treated with TiCl₄ to afford compound 1.

For the preparation of H-10: H-8 (0.25 g, 0.437 mmol, AMRI lot # IN-GUM-D-17), CuI (9.0 mg, 0.043 mmol), Cs₂CO₃ (0.570 g, 1.751 mmol), 1,10-phenathroline (18.0 mg, 0.087 mmol) and H-9 (0.24 g, 1.751 mmol) in butyronitrile (0.2 mL) was stirred at 130° C. for 24 h in sealed tube. The progress of the reaction was monitored by TLC and ultra performance liquid chromatography (UPLC)-mass spectrometry (MS). The reaction mixture was cooled to room temperature and diluted with EtOAc (150 mL), copper salts were filtered through a CELITE™ pad (Sigma Aldrich, St. Louis, MO), washed with excess ethylacetate (25 mL). The filtrate was concentrated under reduced pressure to afford H-10 [0.21 g (crude), AMRI lot # IN-GUM-D-23] as an off white foam solid; ESI-MS: m/z=(M+H)⁺545.22.

For the preparation of compound 1: H-10 (0.210 g, 0.604 mmol, AMRI lot # IN-GUM-D-23) in CH₂Cl₂ (10.0 mL) was charged with TiCl₄ (5.0 mL, 9.064 mmol) at 0° C.

The resulting reaction mixture was stirred at same temperature for 3 h. The progress of the reaction was monitored by UPLC. The reaction was quenched by pouring in ice cold saturated aqueous NaHCO₃ (100 mL), the resulted material was extracted with 10% MeOH in CH₂Cl₂ (3×100 mL). The combined organic layer was washed with sat. aq. NaHCO₃ (3×50.0 mL), brine (5×50.0 mL), dried over Na₂SO₄, filtered, and concentrated under reduced pressure to obtain crude material as a pale yellow solid. The obtained crude material was purified by preparative-HPLC (Method-A). The pure fractions were collected and CH₃CN was concentrated under reduced pressure. Aqueous layer (10.0 mL) was extracted with 10% MeOH in CH₂Cl₂ (3×20 mL), dried over Na₂SO₄, filtered, and concentrated to afford compound 1 [0.020 g, 10.10% (over two steps) AMRI lot # IN-GUM-D-25) as an off-white solid.

¹- H NMR (400 MHz, DMSO): δ 9.38 (s, 1H), 7.44 (s, 5H), 7.15 (d, J=8.4 Hz, 2H), 6.88 (d, J=8.4 Hz, 1H), 6.74 (d, J=8.8 Hz, 3H), 6.67-6.62 (m, 2H), 4.75-4.72 (m, 1H), 3.75-3.73 (m, 2H), 3.63-3.59 (m, 1H), 2.96 (bs, 2H), 2.51 (s, 2H), 2.21-2.16 (m, 1H), 0.91-0.87 (m, 3H), 0.27-0.25 (bs, 2H), 0.14-0.12 (m, 1H), -0.28-−0.30 (m, 1H); ESI-MS: m/z=(M+H)⁺455.16.

### Example 3

This example demonstrates a synthesis of compound 2 in an embodiment of the invention.

FIG. 3 is a chemical scheme of preparing compound 2 from H-8. H-8 was reacted with H-11 in a sealed tube to produce H-12. H-12 was treated with HCl to afford H-13. H-13 was treated with propionaldehyde to achieve H-14. H-14 was treated with TiCl₄ to afford compound 2.

For the preparation of H-12: H-8 (0.300 g, 0.525 mmol, AMRI lot # IN-GUM-D-17), CuI (50 mg, 0.262 mmol), Cs₂CO₃ (0.341 g, 1.050 mmol), 1,10-phenathroline (20.0 mg, 0.105 mmol), and H-11 (0.363 g, 2.101 mmol) in butyronitrile (0.2 mL) was stirred at 130° C. for 24 h in a sealed tube. The progress of the reaction was monitored by TLC and UPLC-MS. Reaction mixture was cooled to room temperature and diluted with EtOAc (150 mL), copper salts were filtered through a CELITE™ pad (Sigma Aldrich, St. Louis, MO), washed with excess ethylacetate (25 mL). The filtrate was washed with sat. aq. NaHCO₃ (2×50 ml), dried over Na₂SO₄, filtered, and concentrated under reduced pressure to obtain crude material. The obtained crude material was purified by flash chromatography using silica gel (100-200 mesh) and eluted with 15-20% EtOAc in hexanes. Combined column fractions were concentrated under reduced pressure to afford H-12 [0.24 g, 75.0%, AMRI lot # IN-GUM-D-24] as a pale yellow oil.

¹H NMR (400 MHz CDC13): δ 7.48-7.45 (m, 4H), 7.43-7.30 (m, 8H), 7.04-7.00 (m, 2H), 6.88 (dd, J=8.4 Hz, 2.0 Hz, 1H), 6.80 (bs, 1H), 6.62 (d, J=8.8 Hz, 2H), 5.08 (s, 2H), 4.87-4.84 (m, 1H), 4.29-4.25 (m, 2H), 4.01-3.96 (m, 2H), 3.75-3.71 (m, 1H), 2.18-2.14 (m, 1H), 1.44 (s, 9H), 0.32-0.26 (bs, 3H), 0.02-0.05 (m, 1H) ; ESI-MS: m/z=(M+H)⁺617.24.

For the preparation of H-13: H-12 (0.250 g, 0.405 mmol, AMRI lot # IN-GUM-D-24) in dioxane (10.0 mL) was charged with HC1 [1.0 mL (4.0 M in 1,4-dioxane)] at 0° C. The resulting reaction mixture was stirred at room temperature for 16 h. The reaction mixture was concentrated under reduced pressure to afford H-13 [0.28 g (crude HCl salt), AMRI lot # IN-GUM-D-33] as a pale yellow oil; ESI-MS: m/z=(M+H)⁺517.18.

For the preparation of H-14: H-13 (0.280 g, 0.542 mmol, AMRI lot # IN-GUM-D-33) in MeOH (10.0 mL) was charged propionaldehyde (0.7 mL, 2.710 mmol) followed by acetic acid (0.1 mL) at room temperature under argon atmosphere. The resultant reaction mixture was stirred for 4 h. NaBH₃CN (0.10 g, 1.620 mmol) was added in two to three lots, stirring was continued for 16 h. The reaction mixture was diluted with CH₂Cl₂ (100 mL), washed the CH₂Cl₂ layer with sat. aq. NaHCO₃ (2×50 mL), brine (2×50 mL), dried over Na₂SO₄, filtered, and concentrated under reduced pressure to afford H-14 [0.210 g (crude), AMRI lot # IN-GUM-D-34] as a pale yellow semi solid; ESI-MS: m/z=(M+H)⁺559.24.

For the preparation of compound 2: H-14 (0.260 g, 0.905 mmol, AMRI lot # IN-GUM-D-56) in CH₂Cl₂ (20.0 mL) was charged with TiCl₄ (4.0 mL) at 0° C. The resulting reaction mixture was stirred at the same temperature for 3 h. The progress of the reaction was monitored by UPLC. The reaction was quenched by pouring in ice cold NaHCO₃ (100 mL), and the resulting material was extracted with 10% MeOH in CH₂Cl₂ (3×100 mL). The combined organic layer was washed with sat.aq. NaHCO₃ (3×50.0 mL), brine (2×30.0 mL), dried over Na₂SO₄, filtered, and concentrated under reduced pressure to obtain crude material. The obtained crude material was purified by preparative-HPLC (Method-A). The pure fractions were collected and CH₃CN was concentrated under reduced pressure. The aqueous layer (10.0 mL) was extracted with 10% MeOH in CH₂Cl₂ (3×20 mL), and the organic layer was washed with sat. aq. NaHCO₃ (10 mL), dried over Na₂SO₄, filtered, and concentrated. The resulting material was lyophilized to afford compound 2 [0.038 g, 20.10% (over three steps) AMRI lot # IN-GUM-D-58] as an off-white solid.

¹H NMR (400 MHz, DMSO): δ 9.42 (s, 1H), 7.45 (s, 5H), 7.15 (d, J=8.4 Hz, 2H), 6.88 (d, J=8.4 Hz, 1H), 6.74 (d, J=8.8 Hz, 3H), 6.67-6.61 (m, 2H), 4.75-4.61 (m, 1H), 3.71-3.68 (m, 2H), 3.64-3.59 (m, 1H), 2.89-2.84 (m, 2H), 2.38-2.32 (m, 2H), 2.21-2.16 (m, 1H), 1.31-1.23 (m, 2H), 0.85-0.81 (m, 3H), 0.28-0.22 (bs, 2H), 0.1-0.10 (m, 1H), −0.28-−0.31 (m, 1H); ESI-MS: m/z=(M+H)⁺469.69.

### Example 4

This example demonstrates a synthesis of compound 6 in an embodiment of the invention.

FIG. 4 is a chemical scheme of preparing compound 6 from H-8. H-8 was reacted with H-15 in a sealed tube to achieve H-16. H-16 was treated with TiCl₄ to afford compound 6.

For the preparation of H-16: H-8 (0.20 g, 0.350 mmol, AMRI lot # IN-GUM-C-197), CuI (7.0 mg, 0.035 mmol), Cs₂CO₃ (0.227 g, 0.700 mmol), 1,10-phenathroline (14.0 mg, 0.070 mmol) and H-15 (0.4 mL mmol) was stirred at 125° C. for 24 h in a sealed tube. The progress of the reaction was monitored by TLC and UPLC-MS. The reaction mixture was cooled to room temperature and diluted with EtOAc (100 mL), copper salts were filtered through a CELITE™ pad (Sigma Aldrich, St. Louis, MO), washed with excess ethylacetate (50 mL), filtrate was washed with sat. aq. NaHCO₃ (2×50 ml), dried over Na₂SO₄, filtered, and concentrated under reduced pressure to afford H-16 [0.21 g (crude), AMRI lot # IN-GUM-C-208] as an off white foam solid; ESI-MS: m/z=(M+H)⁺573.26.

For the preparation of compound 6: H-16 (0.210 g, 0.367 mmol, AMRI lot # IN-GUM-C-208) in CH₂Cl₂ (15.0 mL) was charged with TiCl₄ (2.0 mL) at 0° C. The resulting reaction mixture was stirred at same temperature for 3 h. The reaction was quenched by pouring in ice cold sat. aq. NaHCO₃ (100 mL), and the resulting material was extracted with 10% MeOH in CH₂Cl₂ (3×150 mL). The combined organic layer was washed with sat. aq. NaHCO₃ (2×50.0 mL), brine (2×50.0 mL), dried over Na₂SO₄, filtered, and concentrated under reduced pressure to obtain crude material as a pale yellow solid. The obtained crude material was purified by preparative-HPLC (Method-A). The pure fractions were collected, concentrated under reduced pressure, and the resulting material was lyophilized to afford compound 6 [0.040 g, 23.80% (over two steps) AMRI lot # IN-GUM-D-1] as an off-white solid.

¹NMR (400 MHz, DMSO): δ 9.38 (s, 1H), 7.44 (s, 5H), 7.17 (d, J=8.0 Hz, 2H), 6.87-6.77 (m, 4H), 6.67-6.62 (m, 2H), 4.07 (bs, 2H), 3.64-3.60 (m, 1H), 2.95-2.67 (m, 2H), 2.45-2.33 (m, 4H), 2.21-2.17 (m, 1H), 1.53-1.40 (m, 6H), 0.28-0.25 (bs, 2H), 0.14-0.12 (m, 1H), -0.25-−0.27 (m, 1H); ESI-MS: m/z=(M+H)⁺483.21.

### Example 5

This example demonstrates a synthesis of compound 13 in an embodiment of the invention.

FIG. 5 is a chemical scheme of preparing compound 13 from H-8. H-8 was reacted with H-17 in a sealed tube to provide H-18. H-18 was treated with TiC14 to afford compound 13.

For the preparation of H-18: H-8 (1.00 g, 1.750 mmol, AMRI lot # IN-GUM-D-74), CuI (33.0 mg, 0.170 mmol), Cs₂CO₃ (1.130 g, 3.50 mmol), 1,10-phenathroline (70.0 mg, 0.035 mmol), and H-17 (0.70 g, 5.250 mmol) in butyronitrile (3.0 mL) was stirred at 130° C. for 24 h in a sealed tube. The progress of the reaction was monitored by TLC and UPLC-MS. The reaction mixture was cooled to room temperature and diluted with EtOAc (250 mL), copper salts were filtered through a CELITE™ pad (Sigma Aldrich, St. Louis, MO), washed with excess ethylacetate (50 mL), filtrate was washed with sat. aq. NaHCO₃ (2×50 ml), dried over Na₂SO₄, filtered, and concentrated under reduced pressure to afford H-18 [0.980 g (crude), AMRI lot # IN-GUM-D-84] as a brown oil; ESI-MS: m/z=(M+H)⁺573.22.

For the preparation of compound 13: H-18 (0.90 g, 1.573 mmol, AMRI lot # IN-GUM-D-84) in CH₂Cl₂ (50.0 mL) was charged TiCl₄ (10.0 mL) at 0° C. under argon atmosphere. The resulting reaction mixture was stirred at same temperature for 4 h. The progress of the reaction was monitored by UPLC. The reaction was quenched by pouring in ice cold sat. aq. NaHCO₃ (250 mL), and the resulting material was extracted with 10% MeOH in CH₂Cl₂ (3×150 mL). The combined organic layer was washed with sat. aq. NaHCO₃ (2'50.0 mL), brine (2×50.0 mL), dried over Na₂SO₄, filtered, and concentrated under reduced pressure to obtain crude material as a pale yellow solid. The obtained crude material was purified by preparative-HPLC (Method-A). The pure fractions were collected and CH₃CN was concentrated under reduced pressure. The aqueous layer (10.0 mL) was extracted with 10% MeOH in C₂Cl₂ (3×35 mL). The organic layer was washed with sat. aq. NaHCO₃ (25 mL), dried over Na₂SO₄, filtered, and concentrated. The resulting material was lyophilized to afford compound 13 [0.160 g, 18.95% (over two steps), AMRI lot # IN-GUM-D-88] as an off-white solid.

¹H NMR (400 MHz, DMSO): δ 9.41 (s, 1H), 7.44 (s, 5H), 7.15 (d, J=8.4 Hz, 2H), 6.89-6.78 (m, 4H), 6.67-6.62 (m, 2H), 3.80 (d, J=6.8 Hz, 2H), 3.64-3.60 (m, 1H), 2.60-2.53 (m, 1H), 2.45-2.28 (m, 6H), 2.21-2.17 (m, 1H), 1.92-1.88 (m, 1H), 1.49-1.41 (m, 1H), 1.02-0.98 (m, 3H), 0.27-0.24 (bs, 2H), 0.14-0.06 (m, 1H), −0.26-−0.28 (m, 1H); ESI-MS: m/z=(M+H)⁺483.17.

### Example 6

This example demonstrates a synthesis of compound 14 in an embodiment of the invention.

FIG. 6 is a chemical scheme of preparing compound 14 from H-8. H-8 was reacted with H-19 in a sealed tube to provide H-20. H-20 was treated with TiC14 to afford compound 14.

For the preparation of H-20: H-8 (1.00 g, 1.750 mmol, AMRI lot # IN-GUM-D-74), CuI (33.0 mg, 0.170 mmol), Cs₂CO₃ (1.130 g, 3.50 mmol), 1,10-phenathroline (70.0 mg, 0.035 mmol), and H-19 (1.00 g, 7.00 mmol) in butyronitrile (3.0 mL) was stirred at 130° C. for 24 h in a sealed tube. The progress of the reaction was monitored by TLC and UPLC-MS. The reaction mixture was cooled to room temperature and diluted with EtOAc (250 mL). Copper salts were filtered through a CELITE™ pad (Sigma Aldrich, St. Louis, MO), washed with excess ethylacetate (50 mL), filtrate was washed with sat. aq. NaHCO3 (3×50 ml), dried over Na₂SO₄, filtered, and concentrated under reduced pressure to afford H-20 [1.2 g (crude), AMRI lot # IN-GUM-D-87] as a brown oil; ESI-MS: m/z=(M+H)⁺587.21.

For the preparation of compound 14: H-20 (1.1 g, 1.877 mmol, AMRI lot # IN-GUM-D-87) in C₂Cl₂ (50.0 mL) was charged TiC14 (10.0 mL) at 0° C. under argon atmosphere. The resulting reaction mixture was stirred at same temperature for 4 h. The progress of the reaction was monitored by UPLC. The reaction was quenched by pouring in ice cold sat. aq. NaHCO₃ (350 mL), and the resulting material was extracted with 10% MeOH in C₂Cl₂ (3×200 mL). The combined organic layer was washed with sat. aq. NaHCO₃ (2×100.0 mL), brine (2×100.0 mL), dried over Na₂SO₄, filtered, and concentrated under reduced pressure to obtain crude material as a pale yellow solid. The obtained crude material was purified by preparative-HPLC (Method-A). The pure fractions were collected and CH₃CN was concentrated under reduced pressure. The aqueous layer (10.0 mL) was extracted with 10% MeOH in C₂Cl₂ (3×35 mL), organic layer was washed with sat. aq. NaHCO₃ (25 mL), dried over Na₂SO₄, filtered, and concentrated and the resulted material was lyophilized to afford compound 14 [0.160 g, 18.47% (over two steps), AMRI lot # IN-GUM-D-89] as an off-white solid.

¹H NMR (400 MHz, DMSO): δ 9.43 (bs, 1H), 7.44 (s, 5H), 7.15 (d, J=8.4 Hz, 2H), 6.89-6.78 (m, 4H), 6.67-6.61 (m, 2H), 3.80 (d, J=6.8 Hz, 2H), 3.64-3.60 (m, 1H), 2.60-2.53 (m, 1H), 2.45-2.28 (m, 6H), 2.21-2.17 (m, 1H), 1.92-1.88 (m, 1H), 1.49-1.37 (m, 3H), 0.86-0.83 (m, 3H), 0.28-0.21 (bs, 2H), 0.14-0.06 (m, 1H), −0.26- −0.28 (m, 1H); ESI-MS: m/z =(M+H)⁺497.20.

### Example 7

This example demonstrates a synthesis of compound 16 in an embodiment of the invention.

FIG. 7 is a chemical scheme of preparing compound 16 from H-8. H-8 was reacted with H-21 in a sealed tube to provide H-22. H-22 was treated with TiCl₄ to afford compound 16 as two stereoisomers: Peak-1 (“16-1”) and Peak-2 (“16-2”).

For the preparation of H-22: H-8 (0.50 g, 0.875 mmol, AMRI lot # IN-GUM-D-17), Cul (20.0 mg, 0.087 mmol), Cs₂CO₃ (0.570 g, 3.50 mmol), 1,10-phenathroline (40.0 mg, 0.035 mmol), and H-21 (0.5 mL) in butyronitrile (0.5 mL) was stirred at 130° C. for 24 h in a sealed tube. The progress of the reaction was monitored by TLC. The reaction mixture was cooled to room temperature and diluted with EtOAc (150 mL). Copper salts were filtered through a CELITE™ pad (Sigma Aldrich, St. Louis, MO), washed with excess ethylacetate (50 mL), filtrate was washed with sat. aq. NaHCO₃ (3×50 ml), dried over Na₂SO₄, filtered, and concentrated under reduced pressure to obtained crude material. The obtained crude material was purified by flash chromatography using silica gel (100-200 mesh) and eluted with 2-4% H in CH₂Cl₂. Combined pure column fractions were concentrated under reduced pressure to afford H-22 [0.290 g, 58.00%, AMRI lot # IN-GUM-D-47] as a pale yellow semi solid.

¹H NMR (400 MHz, DMSO): δ 7.47-7.26 (m, 12H), 7.03 (d, J=8.0 Hz, 2H), 6.89-6.87 (m, 1H), 6.80-6.78 (m, 3H), 4.09 (t, J=6.0 Hz, 2H), 3.75-3.70 (m, 2H), 3.01-2.85 (m, 4H), 2.61-2.55 (m, 1H), 2.32-2.25 (m, 1H), 2.18-2.00 (m, 4H), 1.42-1.33 (m, 1H), 1.04 (d, J=6.8 Hz, 3H), 0.28-0.26 (bs, 3H), 0.08-−0.10 (bs, 1H); ESI-MS: m/z=(M+H)⁺573.23.

For the preparation of compound 16 (Peak-1 and Peak-2): H-22 (0.290 g, 0.506 mmol, AMRI lot # IN-GUM-D-47) in C₂Cl₂ (30.0 mL) was charged TiCl₄ (10.0 mL) at 0° C. under argon atmosphere. The resulting reaction mixture was stirred at the same temperature for 3 h. The progress of the reaction was monitored by UPLC. The reaction was quenched by pouring in ice cold sat. aq. NaHCO₃ (150 mL), and the resulting material was extracted with 10% MeOH in CH₂Cl₂ (3×150 mL). The combined organic layer was washed with sat. aq. NaHCO₃ (2×50.0 mL), brine (2×50.0 mL), dried over Na₂SO₄, filtered, and concentrated under reduced pressure to obtain crude material as a pale yellow solid. The obtained crude material was purified by preparative-HPLC (Method-A). The pure fractions were collected and CH₃CN was concentrated under reduced pressure. The aqueous layer (10.0 mL) was extracted with 10% MeOH in CH₂Cl₂ (3×15 mL). The organic layer was washed with sat. aq. NaHCO₃ (15 mL), dried over Na₂SO₄, filtered, and concentrated, and the resulting material was lyophilized to afford compound 16 [0.070 g, 16.58% (over two steps), AMRI lot # IN-GUM-D-49] as an off-white solid.

Compound 16 (0.070 g) was purified by SFC (Method-B) to afford compound 16-Peak-1 (16-1) (0.020 g, 57.14%, AMRI lot # IN-GUM-D-67-Peak-1) as an off-white solid. ¹H NMR (400 MHz, DMSO): δ 9.37 (s, 1H), 7.44 (s, 5H), 7.15 (d, J=8.4 Hz, 2H), 6.89 -6.83 (m, 3H), 6.81-6.763 (bs, 1H), 6.67-6.62 (m, 2H), 4.00 (t, J=6.0 Hz, 2H), 3.64-3.59 (m, 1H), 2.80-2.70 (m, 3H), 2.67-2.54 (m, 2H), 2.21-2.11 (m, 2H), 2.06-2.02 (m, 1H), 1.94-1.88 (m, 1H), 1.27-1.19 (m, 1H), 0.96 (d, J=6.8 Hz, 3H), 0.28-0.21 (bs, 2H), 0.14-0.12 (m, 1H), −0.2-−0.28 (m, 1H); ESI-MS: m/z=(M+H)⁺483.21.

Compound 16-Peak-2 (16-2) [0.024 g, 68.57%, AMRI lot # IN-GUM-D-67-Peak-2] was obtained as an off-white solid. ¹H NMR (400 MHz, DMSO): δ 9.38 (s, 1H), 7.44 (s, 5H), 7.15 (d, J=8.4 Hz, 2H), 6.89-6.83 (m, 3H), 6.81-6.76 (bs, 1H), 6.67-6.62 (m, 2H), 4.00 (t, J=6.0 Hz, 2H), 3.64-3.59 (m, 1H), 2.80-2.70 (m, 3H), 2.67-2.54 (m, 2H), 2.21-2.11 (m, 2H), 2.06-2.02 (m, 1H), 1.94-1.88 (m, 1H), 1.27-1.19 (m, 1H), 0.96 (d, J=6.8 Hz, 3H), 0.28-0.21 (bs, 2H), 0.14-0.12 (m, 1H), −0.25-−0.28 (m, 1H); ESI-MS: m/z=(M+H)⁺483.21.

### Example 8

Starting from H-8, other exemplary compounds of formula (I) can be prepared in an embodiment of the invention.

FIG. 8 is a chemical scheme of preparing compound 5 from H-8.

FIG. 9 is a chemical scheme of preparing compound 12 from H-8.

FIG. 10 is a chemical scheme of preparing compound 15 from H-8.

FIG. 11 is a chemical scheme of preparing compound 17 from H-8.

FIG. 12 is a chemical scheme of preparing compound 18 from H-8.

FIG. 13 is a chemical scheme of preparing compound 20 from H-8.

FIG. 14 is a chemical scheme of preparing compound 21 from H-8.

### Example 9

This example demonstrates a compound of formula (I) reduces ERα transcriptional activities.

In a transcriptional reporter gene assay using MCF7 cells harboring an estrogen response element DNA sequence, whereby binding of an active ERα enables the expression of a green fluorescent protein under the control of an estrogen response element DNA sequence. The reduction in fluorescence was measured and the IC₅₀ value (nM) was determined for various compounds of formula (I) in comparison to known antagonists. The results are set forth in FIG. 15 and Table 5.

TABLE 5

| Formula (I) | | |
|---|---|---|
| or Known | Compound | IC₅₀ (nM) |
| Formula (I) | 16 | 3.85 |
| | 15 | 4.8 |
| | 18 | 13.5 |
| | 14 | 19 |
| | 13 | 21 |
| | 6 | 23.4 |
| | 5 | 49.4 |
| | 12 | 58.8 |
| | 17 | 73.7 |
| | 2 | 130 |
| | 21 | 222 |
| | 1 | 358 |
| | 20 | 500 |
| | 19 | 744 |
| Known | 4-hydroxytamoxifen (4OHT) | 0.8 |
| | RU39411 [CAS 120382-04-9] | 0.22 |
| | lasofoxifene (Laso) | 0.2 |
| | fulvestrant (ICI) | 0.35 |
| | GDC0927 [CAS 1642297-01-5] | 0.03 |
| | OP1074 [CAS 1443752-76-8] | 0.17 |
| | bazedoxifene (BZA) | 0.80 |
| | AZD9496 [CAS 1639042-08-2] | 1.53 |
| | raloxifene (RAL) | 0.11 |
| | LSZ102 [2135600-76-7] | 0.34 |
| | brilanestrant (GDC0810) | 4.5 |
| | PIP | 0.93 |
| | RU58668 [CAS 151555-47-7] | 0.92 |

### Example 10

This example demonstrates the influence of a compound of formula (I) on ERα cellular stability.

T47D breast cancer cell lines were generated with stable Tet-ON wild type (WT), Y537S mutation, or D538G halo-ERα mutation and enriched through flow sorting. Cells were cultured for 48 hours in media supplemented with charcoal-stripped fetal bovine serum (FBS) in puromycin. Subsequently, 1 μg/mL doxycycline was added to induce expression and 1 μM G618 antibody was added to follow the expression of the halo-tagged proteins. Cells were then treated with increasing concentrations of a compound of interest between 0 and 10 μM for 24 hours. After treatment, cells were imaged using an INUCYTE™ S3 (Essen BioScience, Ann Arbor, MI). ERα degradation was quantified by using the red channel integrated intensity per image normalized to phase channel confluence area.

Molecules that enhance ERα levels will show an increased signal at higher concentrations and are more selective estrogen response modulator (SERM) like (e.g., 4OHT, which is the active metabolite of tamoxifen). Those that decrease are more selective estrogen receptor degrader (SERD) like (e.g., fulvestrant (ICI)). The data are shown in FIG. 16A (known antagonists) and FIG. 16B (compounds of formula (I) plus 17-beta-estradiol (E2) and 4-hydroxytamoxifen (4OHT)). Tissue specific activities often correlate with a molecule's influence on ERα levels. The data in FIG. 16B demonstrate that a compound of formula (I) can have a range of impacts on receptor levels.

### Example 11

This example demonstrates that exemplary compounds of formula (I) inhibit cancer cell growth.

Samples were tested for cytotoxicity in a live-cell assay of ERα expression in T47D breast cancer cells. Cellular proliferation was followed for 150 hours after treatment with 1 μM and 10 μM of select compounds of formula (I) in the presence of 1 nM hormone (E2) to stimulate proliferation compared to known antiestrogens.

FIGS. 17A-17N demonstrate the cytotoxicity of the compounds of formula (I). Overall, compounds of formula (I) were mostly neutral, like lasofoxifene, but increased ERα levels similar to tamoxifen and higher doses. Only compound 1 showed a weak SERD-like profile.

FIG. 18 shows the results of a crystal violet endpoint assay of MC7 (breast cancer cell line) cellular viability for dimethylsulfoxide (DMSO) (control), E2 (1 nM), and compounds 1, 12, 13, 14, and 16-1 at concentrations of 1 nM, 10 nM, 50 nM, 100 nM, 500 nM, and 1 μM. A crystal violet endpoint assay is useful for obtaining quantitative information about the relative density of cells adhering to multi-well cluster dishes. The crystal violet dye stains DNA. Upon solubilization, the amount of dye taken up by the monolayer can be quantified in a spectrophotometer or plate reader when the absorbance of each well is read at 570 nm. A crystal violet assay kit to measure cell viability is commercially available (e.g., Abcam, Cambridge, MA). All antagonist treatments were performed in the presence of 1 nM hormone (E2) for 1 week. FIG. 18 shows similar trends to the T47D experiments. However, compounds 13 and 14 appear to induce apoptosis at 500 nM.

The data in FIGS. 17A-17N and FIG. 18 demonstrate that a compound of formula (I) antagonizes ERα transcriptional activities in MCF7 breast cancer cells and inhibits E2-stimulated proliferation in T47D and MCF7 breast cancer cell lines.

### Example 12

This example demonstrates that exemplary compounds of formula (I) inhibit cancer cell growth.

The abilities of exemplary compounds of formula (I) to inhibit the cellular proliferation of MCF7 breast cancer cells were measured. Cells were serum-starved for 48 hours before they were treated with either vehicle (DMSO), 1 nM hormone (estradiol [E2]), or 1 nM E2+1 μM compound in triplicate for 150 hours. FIG. 19 shows a summary of the fold-change in cell count versus starting cell count at the last time-point. These data show that a propylazetidine (e.g., compound 4) improved anti-proliferative activities compared to an ethylazetidine (e.g., compound 3). Fluoropropylazetidine (e.g., compound 30) or chloropropylazetidine (e.g., compound 31) side-arms provided similar anti-proliferative activities compared to the propylazetidine of compound 4. Chiral separation of compound 4 (compounds 4A and 4B) modestly improved anti-proliferative activities although neither species showed clearly improved activities. Removing the hydroxyl group from the core ablated anti-proliferative activities (compound 34). Lengthening the alkyl group attached to the pyrrolidine (compounds 22 and 23) did not enhance anti-proliferative activities compared to compound 14. At the R³ position, ortho substitutions (compounds 105 and 116) did not improve anti-proliferative activities compared to compound 14. However, meta substitutions (compounds 111 and 121) reduced anti-proliferative activities compared to compound 14.

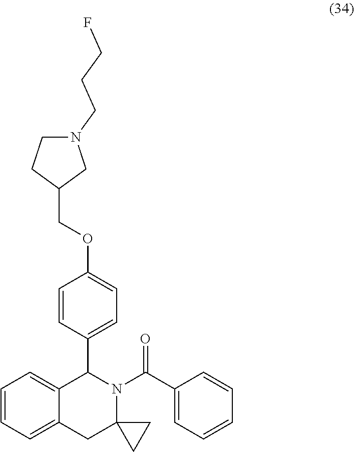 [SMILES: O=C(c1ccccc1)N1C(c2ccc(OCC3CCN(CCCF)C3)cc2)c2ccccc2CC12CC2]

Replacing the ether linkage at X² with either methyl (compounds 24 and 25) or amino (compound 26) groups reduced anti-proliferative activities compared to compound 14. At this site, a thioether attachment (compound 27) did not show an appreciable difference in anti-proliferative activities compared to compound 14. Substitution of a fluoropropyl group (compound 29) compared to the propyl of compound 14 enhanced anti-proliferative activities at 1 μM. Reducing the cyclopropyl group on position R¹ to a methyl (compounds 35(R) and 35(S)) modestly reduced the anti-proliferative activities compared compound 29. Importantly, the fluoropropyl enabled an additional chiral separation into 4 discrete species (compounds 29P1A, 29P1B, 29P2A, and 29P2B), and these molecules equally showed enhanced anti-proliferative activities compared to compound 29.

The abilities of compounds 29P1A, 29P1B, 29P2A, and 29P2B to inhibit the proliferation of T47D breast cancer cell line were measured. Cells were serum-starved for 48 hours before they were treated with either vehicle (Veh), 1 nM E2, or 1 nM E2 +compound for 150 hours. Cells were treated with 10, 100, and 1,000 nM compound alongside lasofoxifene (Laso) a selective estrogen receptor modulator known to possess significant anti-proliferative activities. These data show that each compound is at least as anti-proliferative as Laso at these concentrations. FIG. 20 shows the final average cell count at the end of the experiment for the T47D cells. The anti-proliferative properties of the antagonists in MCF7:WS8 cells, a highly hormone-sensitive model of breast cancer compared to 4OHT were also evaluated. FIG. 21 shows the results of this study as the mean of three replicates±standard deviation. These data show that compounds 29P1A, 29P1B, 29P2A, and 29P2B are similarly anti-proliferative to 4OHT at 1, 100, and 10,000 nM.

Selective estrogen receptor modulator (SERM) treatment leads to an accumulation of ERα in breast cancer cells. Selective estrogen receptor degraders (SERDs) induce receptor a greater receptor degradation than that observed for hormone treatment. SERMs possess tissue-specific agonism and antagonism while SERDs are pure antagonists. An in-cell western approach was used to understand whether the separated compound 29 peaks exhibited SERM or SERD-like profiles in MCF7 breast cancer cells. Cells were serum-starved for 48 hours then treated with either 10 nM or 1 μM E2, fulvestrant (ICI, SERD), 4-hydroxytamoxifen (4OHT, SERM), or compounds 29P1A, 29P1B, 29P2A, or 29P2B for 24 hours. These experiments were performed identically to a previously published report (Zhao et al., Cancer research, 77, 5602-5613 (2017)). FIG. 22 shows the measured ERα levels, normalized to cell count in each well. Overall, the peaks were observed to be SERM-like without a reduction in observed ERα levels after treatment.

Hormone-activated ERα associates with coactivator proteins to form an epigenetically activating complex (e.g., SRC3 and p300). SERMs and SERDs favor an ERα conformation that sterically precludes coactivator association. As such, inhibiting the association between ERα and coactivators is an aspect of therapeutic antagonistic anticancer activities. To study this, the NANOBIT™ split luciferase system (Promega, Madison, WI) was used to measure the abilities of 4OHT, compound 29P1A, and compound 29P1B to inhibit the binding of the nuclear recognition domain of the coactivator SRC3 to ERα in HEK293T cells. Cells were serum-starved for 48 hours before they were treated with 10, 50, 100 and 500 nM in the presence of 1 nM E2. The signal was normalized to cell count in each well by label-free cell counting. FIG. 23 shows results from this experiment as a mean of 3 replicates±standard deviation. At each concentration, compounds 29P1A and 29P1B showed similar or improved inhibitory activities compared to 4OHT, indicating that these molecules antagonize hormone-stimulated co-activator binding.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of formula (I) or a pharmaceutically acceptable salt thereof: 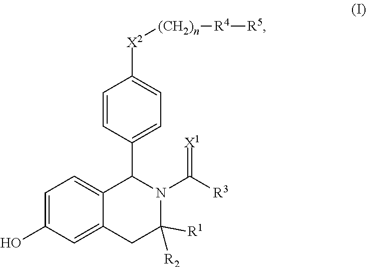
wherein
X¹ is O or S;
X² is a bond, alkenyl, O, S, or NR₆;
R¹ is selected from H and C₁₋₆ alkyl;
R² is C₁₋₆ alkyl; or
R¹ and R² together form C₃₋₆ cycloalkyl,
R³ is C₂₋₁₂ alkyl or phenyl optionally substituted with at least one substituent selected from C₁₋₆ alkyl, C₃₋₆ cycloalkyl, hydroxy, alkoxy, cycloalkoxy, halo, and amino; or
R³ is phenyl with two substituents that together with the phenyl form an optionally substituted bicyclic nitrogen-containing heteroaryl;
R⁴ is a nitrogen-containing C₃₋₇ heterocycloalkyl, —NR⁷—, or —C(═O)O—;
R⁵, R⁶, and R⁷ are the same or different and each is a hydrogen or C₁₋₆ alkyl; and
n is 0 or an integer of 1 to 5,
wherein the C₁₋₆ alkyl and C₃₋₆ cycloalkyl can be substituted with one or more substituents selected from hydroxy, halo, alkoxy, alkyl, cycloalkyl, heterocycloalkyl, aryl, heteroaryl, amino, alkylamino, dialkylamino, and carboxylato.

2. The compound of claim 1 or a pharmaceutically acceptable salt thereof, wherein X¹ and X² are both O.

3. The compound of claim 1 or a pharmaceutically acceptable salt thereof, wherein R¹ and R² together form cyclopropyl.

4. The compound of claim 1 or a pharmaceutically acceptable salt thereof, wherein R³ is neopentyl.

5. The compound of claim 1 or a pharmaceutically acceptable salt thereof, wherein R³ is phenyl optionally substituted with at least one substituent selected from C₁₋₆ alkyl, haloalkyl, and halo.

6. The compound of claim 1 or a pharmaceutically acceptable salt thereof, wherein R³ is substituted phenyl, wherein two substituents together with the phenyl group form indazolyl, indolizinyl, pyrazolo[1,5-a]pyridinyl, or imidazo[1,5-a]pyridinyl, each of which is optionally substituted.

7. The compound of claim 1 or a pharmaceutically acceptable salt thereof, wherein R⁴ is 3-azetidinyl, 1-pyrrolidinyl, 3-pyrrolidinyl, 1-piperidinyl, 4-piperidinyl, 1-piperazinyl, or 1-azepanyl.

8. The compound of claim 1 or a pharmaceutically acceptable salt thereof, wherein R⁴ is —NH— or —N(C₁₋₃ alkyl)—.

9. The compound of claim 1 or a pharmaceutically acceptable salt thereof, wherein R⁵ is C₁₋₃ alkyl.

10. The compound of claim 1 or a pharmaceutically acceptable salt thereof, wherein n is 0 or 2.

11. The compound of claim 1, wherein the compound of formula (I) is 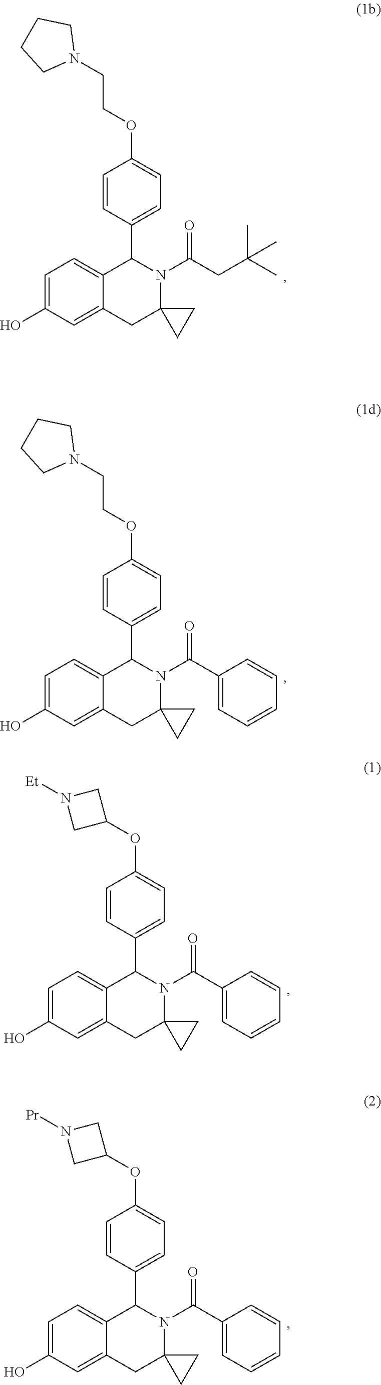 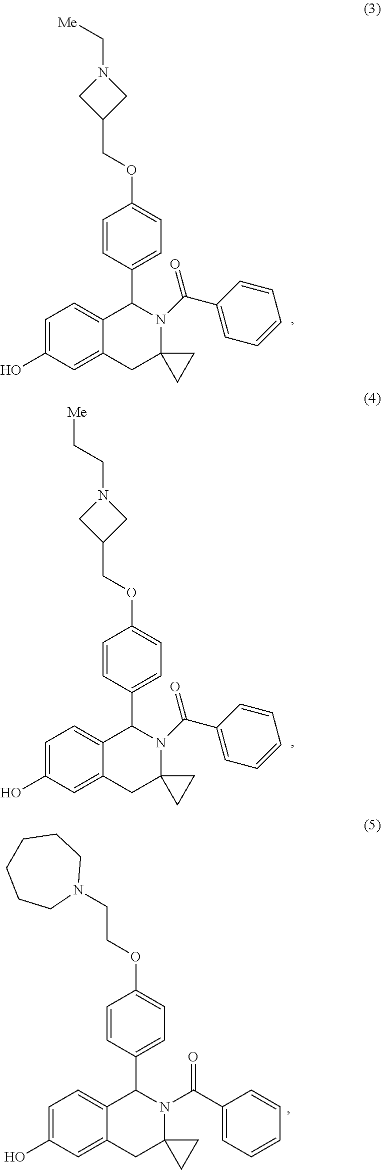 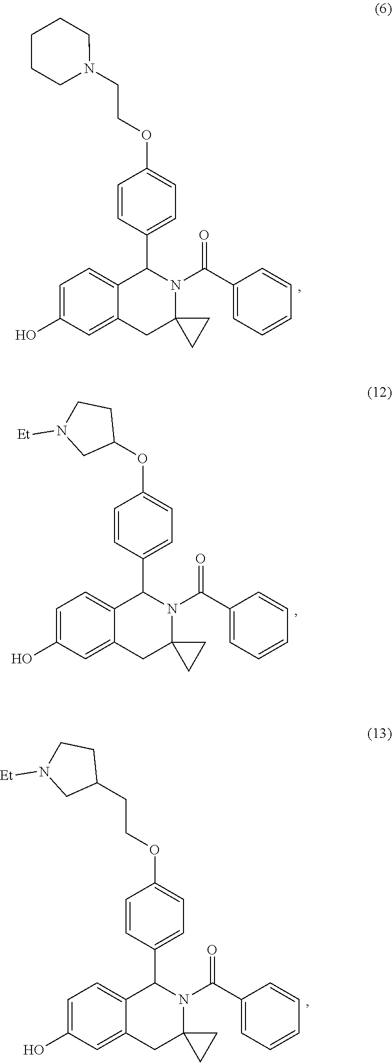 [SMILES: O=C(c1ccccc1)N1C(c2ccc(OCCN3CCCCC3)cc2)c2ccc(O)cc2CC12CC2 ; CCN1CCC(Oc2ccc(C3c4ccc(O)cc4CC4(CC4)N3C(=O)c3ccccc3)cc2)C1 ; CCN1CCC(CCOc2ccc(C3c4ccc(O)cc4CC4(CC4)N3C(=O)c3ccccc3)cc2)C1] 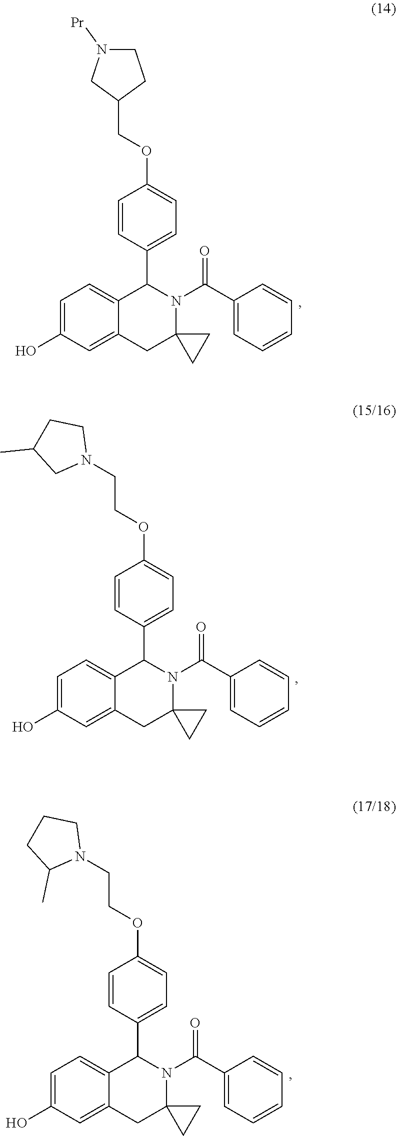 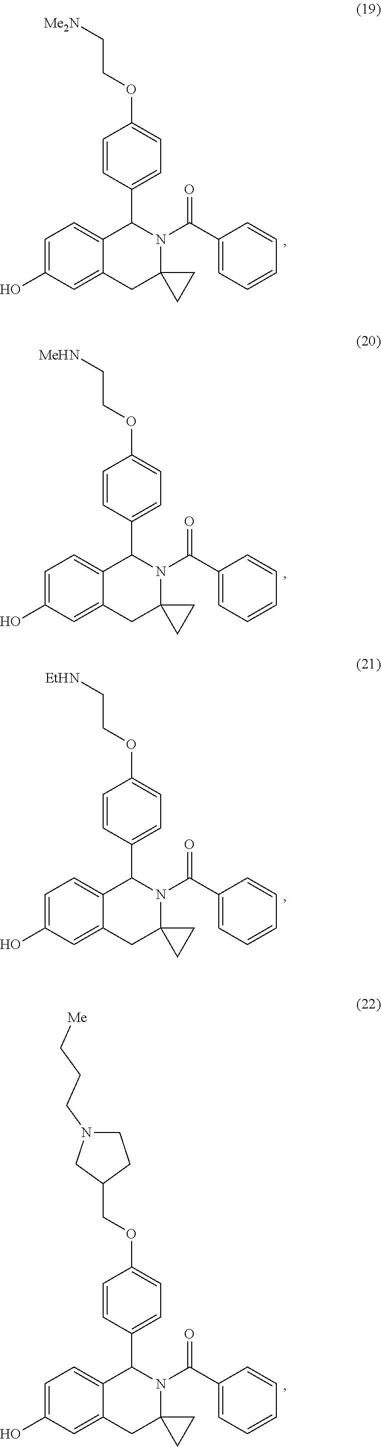 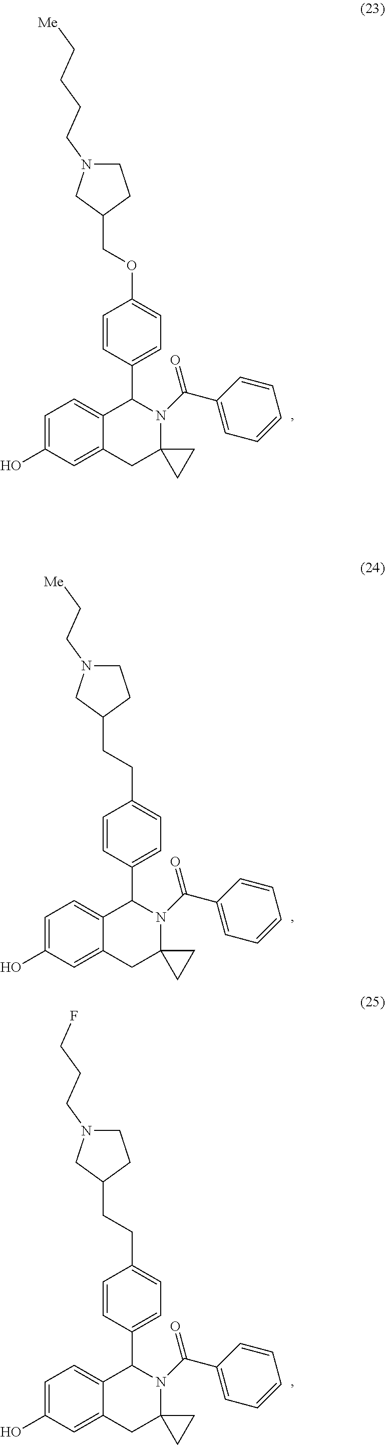 [SMILES: CCCCCN1CCC(COc2ccc(C3c4ccc(O)cc4CC4(CC4)N3C(=O)c3ccccc3)cc2)C1 ; CCCN1CCC(CCc2ccc(C3c4ccc(O)cc4CC4(CC4)N3C(=O)c3ccccc3)cc2)C1 ; O=C(c1ccccc1)N1C(c2ccc(CCC3CCN(CCCF)C3)cc2)c2ccc(O)cc2CC12CC2] 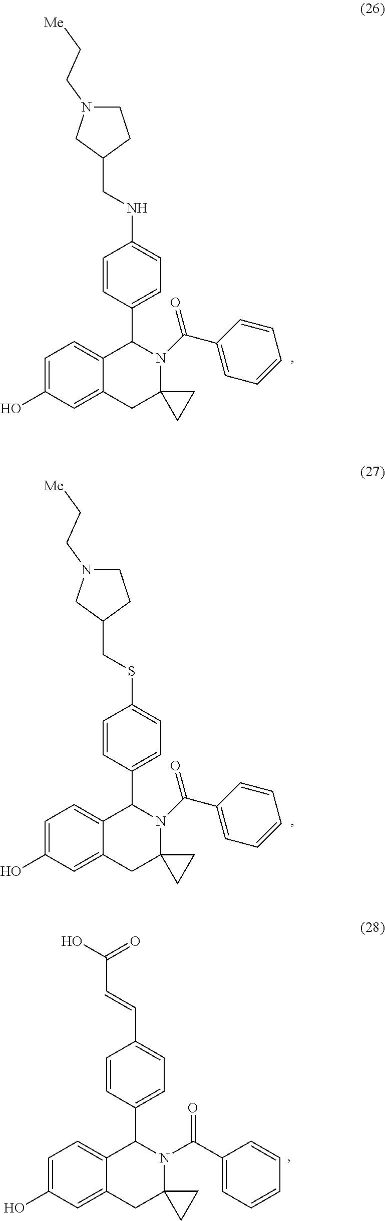 [SMILES: CCCN1CCC(CNc2ccc(C3c4ccc(O)cc4CC4(CC4)N3C(=O)c3ccccc3)cc2)C1 ; CCCN1CCC(CSc2ccc(C3c4ccc(O)cc4CC4(CC4)N3C(=O)c3ccccc3)cc2)C1 ; O=C(O)/C=C/c1ccc(C2c3ccc(O)cc3CC3(CC3)N2C(=O)c2ccccc2)cc1] 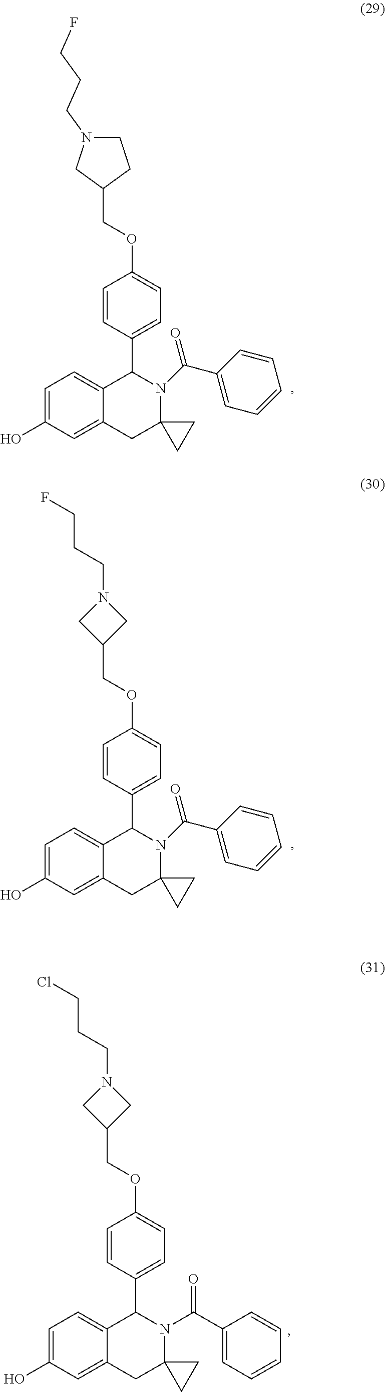 [SMILES: O=C(c1ccccc1)N1C(c2ccc(OCC3CCN(CCCF)C3)cc2)c2ccc(O)cc2CC12CC2 ; O=C(c1ccccc1)N1C(c2ccc(OCC3CN(CCCF)C3)cc2)c2ccc(O)cc2CC12CC2 ; O=C(c1ccccc1)N1C(c2ccc(OCC3CN(CCCCl)C3)cc2)c2ccc(O)cc2CC12CC2] 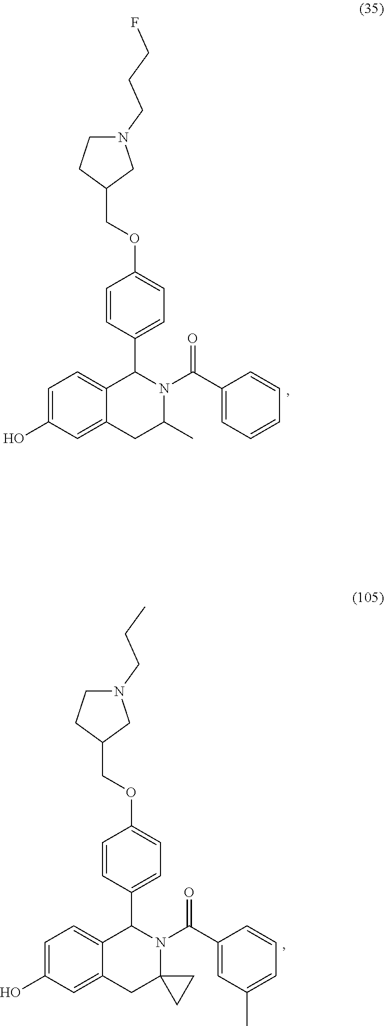 [SMILES: CC1Cc2cc(O)ccc2C(c2ccc(OCC3CCN(CCCF)C3)cc2)N1C(=O)c1ccccc1 ; CCCN1CCC(COc2ccc(C3c4ccc(O)cc4CC4(CC4)N3C(=O)c3cccc(C)c3)cc2)C1] 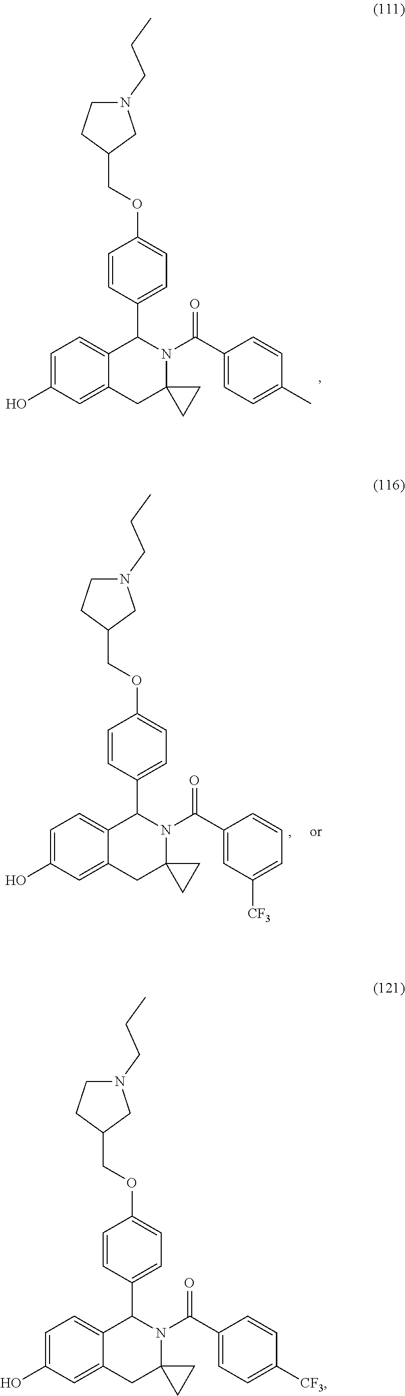 [SMILES: CCCN1CCC(COc2ccc(C3c4ccc(O)cc4CC4(CC4)N3C(=O)c3ccc(C)cc3)cc2)C1 ; CCCN1CCC(COc2ccc(C3c4ccc(O)cc4CC4(CC4)N3C(=O)c3cccc(C(F)(F)F)c3)cc2)C1 ; CCCN1CCC(COc2ccc(C3c4ccc(O)cc4CC4(CC4)N3C(=O)c3ccc(C(F)(F)F)cc3)cc2)C1]
or a stereoisomer thereof and/or a pharmaceutically acceptable salt thereof.

12. A pharmaceutical composition comprising a compound of claim 1 or a pharmaceutically acceptable salt thereof and a pharmaceutically acceptable carrier.

13. A method of treating an estrogen-mediated disease requiring inhibition of estrogen receptor alpha (ERα) comprising administering to a subject in need thereof the compound of claim 1 or a pharmaceutically acceptable salt thereof.

14. The method of claim 13, wherein the estrogen-mediated disease is an ER-positive cancer, osteoporosis, vulvovaginal atrophy, hormone replacement therapy, one or more symptoms of menopause, obesity, or a fibroid.

15. The method of claim 14, wherein the estrogen-mediated disease is an ER-positive cancer selected from breast cancer, ovarian cancer, colon cancer, prostate cancer, lung cancer, and endometrial cancer.

16. The method of claim 13, wherein the compound of formula (I) is used in combination with at least one other therapeutic agent.

17. The method of claim 16, wherein the at least one other therapeutic agent is a hormonal agent or an anti-cancer agent selected from a CDK4/6 inhibitor, an anti-cancer hormonal agent, an aromatase inhibitor, and a combination thereof

18. A method of inhibiting estrogen receptor alpha (ERα) in a cell comprising contacting the cell with the compound of claim 1 or a pharmaceutically acceptable salt thereof.

19. The method of claim 18, wherein inhibiting ERa in the cell treats an estrogen-mediated disease in a subject in need thereof selected from an ER-positive cancer, osteoporosis, vulvovaginal atrophy, hormone replacement therapy, one or more symptoms of menopause, obesity, and a fibroid.

20. The method of claim 19, wherein the estrogen-mediated disease is an ER-positive cancer selected from breast cancer, ovarian cancer, colon cancer, prostate cancer, lung cancer, and endometrial cancer.
